(19) United States
(12) Patent Application Publication
(10) Pub. No.: US 2026/0207743 A1
(43) Pub. Date: Jul. 23, 2026

(54) Compositions and Methods for Treating Metastatic Cancer

(71) Applicant: The University of Chicago, Chicago, IL (US)

(72) Inventors: Ralph Weichselbaum, Chicago, IL (US); Hua Liang, Chicago, IL (US); Andras Piffko, Chicago, IL (US); Sean Pitroda, Chicago, IL (US); Connor Lynch, Chicago, IL (US)

(21) Appl. No.: 19/455,723

(22) Filed: Jan. 21, 2026

Related U.S. Application Data
(60) Provisional application No. 63/747,746, filed on Jan. 21, 2025.

(51) Int. Cl. A61K 39/395; A61K 31/5377; A61K 39/00; A61K 51/00; A61P 35/04; G01N 33/575

(52) U.S. Cl. CPC A61K 39/3955; A61K 31/5377; A61K 51/00; A61P 35/04; G01N 33/5758; A61K 2039/505; A61K 2121/00; G01N 2333/485; G01N 2333/70503; G01N 2333/71

(57) ABSTRACT

The present disclosure relates to compositions and methods for treating metastatic cancer.

### CROSS-REFERENCE TO RELATED APPLICATIONS

This application claims the benefit of U.S. Provisional Application 63/747,746, filed Jan. 21, 2025, which is incorporated by reference herein in its entirety.

### STATEMENT REGARDING FEDERALLY FUNDED RESEARCH

This invention was made with government support under CA262508 awarded by the National Institutes of Health. The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Jan. 20, 2026, is named “25-0073-US_ST26.xml” and is 4,615 bytes in size.

### FIELD OF THE DISCLOSURE

The disclosure is directed to compositions and methods for treating tumors and metastatic cancer. This disclosure includes compositions for inhibiting amphiregulin (AREG), epidermal growth factor receptor (EGFR), Cluster of Differentiation 47 (CD47), immune checkpoint proteins, and/or other related proteins; with or without radiotherapy (RT).

### BACKGROUND

Radiotherapy (RT) plays a critical role in cancer treatment and approximately 50-60% of all cancer patients receive RT during their treatment course¹. Ionizing radiation (IR) causes cell death principally through the induction of DNA damage and immune-mediated anti-tumor effects such as dendritic cell (DC)-mediated T-cell priming²⁻⁴. However, RT-induced immunosuppressive factors such as the attraction of regulatory T cells (Treg) and myeloid-derived suppressor cells (MDSCs) or production of suppressive cytokines, negatively impact RT outcomes⁵⁻⁷.

RT is primarily used to treat localized tumors, but there is increasing interest in its use as a potentially curative treatment of oligometastatic disease or in patients with widespread metastases in combination with immune checkpoint blockade (ICB)^{8,9}. Despite the potentially synergistic immune-activating mechanisms of RT and ICB, clinical trials testing these interactions in patients with metastatic disease have not met their primary endpoints^{10,11}. We hypothesized that the lack of treatment efficacy in these trials is due to factors induced by RT that promote distant tumor growth, which we have termed the “badscopal” effect¹².

The epidermal growth factor receptor (EGFR) is activated by amphiregulin (AREG) through dimerization and autophosphorylation^{13,14}, and is an integral part of type 2 immune-mediated tolerance and resistance mechanisms in various immune cells¹⁵⁻¹⁹. AREG overexpression has been found in a wide variety of human cancers¹⁸ and has been associated with intestinal recovery following RT in preclinical models²⁰. However, the role of tumor-cell derived AREG in response to RT remains unclear.

### SUMMARY OF THE DISCLOSURE

It is against the above background that the present disclosure provides certain advantages over the prior art.

In a first aspect, the present disclosure provides a composition for treating metastatic cancer in a subject in need thereof, including:
a) a therapeutically effective amount of an amphiregulin (AREG) inhibitor; and
b) a therapeutically effective amount of a radiotherapy agent.

In some embodiments of the first aspect, the AREG inhibitor comprises one or more of an anti-AREG antibody or an antigen-binding fragment thereof, an anti-AREG antibody drug conjugate (ADC), a protein binder, a peptide, an RNA, an AREG siRNA, a small molecule, and heparin. In some embodiments the AREG inhibitor comprises an anti-AREG antibody or an antigen-binding fragment thereof.

In some embodiments of the first aspect, the radiotherapy agent comprises one or more of yttrium-90, iodine-131, samarium-153, lutetium-177, astatine-211, lead-212 with bismuth-212, radium-223, actinium-225, and thorium-227.

In some embodiments of the first aspect, the composition further includes a therapeutically effective amount of an epidermal growth factor receptor (EGFR) inhibitor. In some embodiments of the first aspect, the EGFR inhibitor comprises one or more of an anti-EGFR antibody or an antigen-binding fragment thereof, an anti-EGFR ADC, a protein binder, a peptide, an RNA, an EGFR siRNA, and a small molecule. In some embodiments of the first aspect, the EGFR inhibitor is a small molecule tyrosine kinase inhibitor (TKI) of the tyrosine kinase domain of EGFR (EGFR TKI). In some embodiments of the first aspect, the EGFR inhibitor is gefitinib.

In some embodiments of the first aspect, the composition further includes a therapeutically effective amount of a Cluster of Differentiation 47 (CD47) inhibitor. In some embodiments of the first aspect, the CD47 inhibitor is one or more of an anti-CD47 antibody or an antigen-binding fragment thereof, an anti-CD47 ADC, a protein binder, a peptide, an RNA, a CD47 siRNA, and a small molecule.

In some embodiments of the first aspect, the composition further includes a therapeutically effective amount of a Signal transducer and activator of transcription 3 (STAT3) inhibitor.

In some embodiments of the first aspect, the composition further includes a therapeutically effective amount of a Signal regulatory protein α (SIRPα) inhibitor.

In a second aspect, the present disclosure provides a composition for treating metastatic cancer in a subject in need thereof, comprising:
a) a therapeutically effective amount of a tumor necrosis factor α converting enzyme (TACE) inhibitor; and
b) a therapeutically effective amount of a radiotherapy agent.

In some embodiments of the second aspect, the composition further includes a therapeutically effective amount of an AREG inhibitor.

In a third aspect, the present disclosure provides a composition for treating metastatic cancer in a subject in need thereof, comprising:
a) a therapeutically effective amount of an AREG inhibitor; and
b) a therapeutically effective amount of an immune checkpoint inhibitor.

In some embodiments of the third aspect, the immune checkpoint inhibitor is an antibody or an antigen binding fragment thereof that binds to PD-1, PD-L1, CTLA-4, or CD47.

In some embodiments of the third aspect, the composition further includes a therapeutically effective amount of a radiotherapy agent.

In a fourth aspect, the present disclosure provides a composition for treating metastatic cancer in a subject in need thereof, comprising:
a) a therapeutically effective amount of an AREG inhibitor; and
b) a therapeutically effective amount of an epidermal growth factor receptor (EGFR) inhibitor.

In a fifth aspect, the present disclosure provides a method for treating a tumor in a patient, comprising:
a) obtaining a sample from the patient;
b) performing an assay on the sample to determine a level of AREG in the sample;
c) determining that the AREG level is higher than a reference level; and
d) administering a therapeutically effective amount of an AREG inhibitor to the patient;
and/or
e) administering a therapeutically effective amount of a radiotherapy agent to the patient,
wherein the tumor is treated.

In some embodiments of the fifth aspect, the sample is one or more of blood, plasma, and tissue. In some embodiments of the fifth aspect, the sample is plasma.

In some embodiments of the fifth aspect, step d) comprises administering a therapeutically effective amount of an AREG inhibitor to the patient and administering a therapeutically effective amount of a radiotherapy agent to the patient.

In some embodiments of the fifth aspect, the method further includes:
e) performing an assay on the sample to determine a level of circulating plasma EGFR in the sample;
f) determining that the EGFR level is higher than a reference level; and
g) administering a therapeutically effective amount of an EGFR inhibitor to the patient.

In some embodiments of the fifth aspect, the method further includes:
e) performing an assay on the sample to determine a level of circulating plasma CD47 in the sample;
f) determining that the CD47 level is higher than a reference level; and
g) administering a therapeutically effective amount of an CD47 inhibitor to the patient.

In a sixth aspect, the present disclosure provides a method for treating a tumor in a patient, including:
obtaining a sample from the patient;
performing an assay on the sample to determine the level of circulating plasma AREG in the patient;
determining that the AREG level is higher than a reference level;
administering a therapeutically effective amount of an AREG inhibitor to the patient; and
administering a therapeutically effective amount of an immune checkpoint inhibitor to the subject,
whereby the tumor is treated.

In a seventh aspect, the present disclosure provides a method for treating cancer in a patient, including:
obtaining a sample from the patient;
performing an assay on the sample to determine the level of circulating plasma AREG in a subject;
determining that the AREG level is higher than a reference level;
performing an assay on the sample to determine the level of circulating plasma EGFR in a subject;
determining that the EGFR level is higher than a reference level;
administering a therapeutically effective amount of an AREG inhibitor to the patient; and
administering a therapeutically effective amount of an EGFR inhibitor to the patient,
whereby the cancer is treated.

In an eighth aspect, the present disclosure provides a method for treating a tumor in a patient, including:
determining if an AREG level in the patient is higher than a reference level by:
   a) obtaining a sample from the patient;
   b) performing an assay on the sample to determine the level of circulating plasma AREG in the patient;
if the sample has an AREG level higher than the reference level, then administering a therapeutically effective amount of an AREG inhibitor and a therapeutically effective amount of a radiotherapy agent to the patient, or
if sample has an AREG level lower than the reference level, then administering a therapeutically effective amount of a radiotherapy agent to the patient.

In some embodiments of the eighth aspect, the method further includes:
c) determining if an EGFR level is higher than a reference level by performing an assay on the sample to determine the level of circulating plasma EGFR in the patient;
if the sample has an EGFR level higher than the reference level, then administering a therapeutically effective amount of an EGFR inhibitor to the patient.

In some embodiments of the eighth aspect, the method further comprises:
c) determining if a CD47 level is higher than a reference level by performing an assay on the sample to determine the level of circulating plasma CD47 in the patient;
if the sample has a CD47 level higher than the reference level, then administering a therapeutically effective amount of a CD47 inhibitor to the patient.

In some embodiments of the first, sixth, seventh, and eighth aspects and embodiments thereof, the tumor is a primary tumor or a metastatic tumor.

In a ninth aspect, the present disclosure provides a kit, including:
a) a first assay for determining a concentration of AREG in a sample; and
b) a second assay for determining a concentration of EGFR in the sample; and/or
c) a third assay for determining a concentration of CD47 in the sample.

In some embodiments of the ninth aspect, the first assay, second assay, and/or third assay each independently comprises an ELISA, a sandwich immunoassay with electrochemiluminescence, a bead-based immunoassay, and/or a proximity extension assay.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIGS. 1A-1H: Radiotherapy induced amphiregulin (AREG) expression and growth of distant metastasis. FIG. 1A, Shows a schematic of the biopsy acquisition and gene expression analysis. Genes that are (i) induced by stereotactic body radiotherapy (SBRT) and (ii) correlate with distant tumor growth are highlighted (left). Pearson correlation coefficient of 60 genes: distant tumor progression based on RECIST criteria was correlated with gene expression post-SBRT in a microarray of n=22 matched pre- and post-SBRT biopsies (right). FIG. 1B shows a Kaplan-Meier plot showing overall (left) and progression-free survival (right), stratified by increase or decrease of AREG-expression post-SBRT (Log-rank [Mantel-Cox] test). FIG. 1C shows a tumor growth curve of LLC flank tumors±IR. Flank tumors were irradiated at 150 mm³ volume (vol) with indicated doses. Two-way ANOVA, Tukey's multiple comparison test. FIG. 1D shows a number of lung metastases per section (left), and mean size of the five largest metastases/lung normalized to lung area (right). One-way ANOVA, Šídák's multiple comparisons test. FIG. 1E shows a representative H&E stainings of lungs. Black arrows indicate metastatic lesions. FIG. 1F shows a tumor growth curve of LLC^{AR+} and LLC^{AR-} flank tumors±20 Gy. Two-way ANOVA, Tukey's multiple comparison test. FIG. 1G shows a number of lung metastases per section (left), and mean size of the five largest metastases/lung normalized to lung area (right). FIG. 1H shows representative H&E stainings of lungs at experimental endpoints. Black arrows indicate metastatic lesions. Two sections at a distance of 300 μm were analyzed per lung for FIG. 1D and FIG. 1G, two independent experiments pooled for FIGS. 1C-1D, one representative experiment shown for FIGS. 1F-1G. Data are presented as mean±SEM.

FIGS. 2A-2L: Radiation-induced AREG modulated the myeloid immune landscape in NSCLC patient PBMCs and a murine lung metastasis model. FIG. 2A shows spectral flow cytometry. t-SNE clustering of 800,000 live CD45⁺ PBMCs of 30 matched pre- and post-SBRT samples. FIG. 2B shows expression intensity of Tyr992 pEGFR (left) and identification of the two clusters with highest pEGFR expression (right) projected on t-SNE. FIG. 2C shows mean fluorescence intensity (MFI) of Tyr992 pEGFR in manually-gated immune cell populations, samples pre- and post-SBRT combined. One-way ANOVA, Šídák's multiple comparisons test. FIG. 2D shows density plots of t-SNE from PBMCs pre- and post-SBRT; pEGFR⁺ CD33⁺ CD14⁺ population outlined. FIG. 2E shows an assay where PBMC samples were trichotomized by fold change of the CD33⁺ CD14⁺ population (% of live CD45⁺) pre- and post-SBRT (left); high (2,0-4,5-fold increase), medium (1,1-1,9-fold increase), and low (0,6-1,1-fold change) (Log-rank [Mantel-Cox] test). FIG. 2F shows a Kaplan Meier plot showing overall survival of trichotomized patient groups. FIG. 2G shows spectral flow cytometry. t-SNE clustering of 230,000 live CD45⁺ immune cells from lungs of non-tumor-bearing and tumor-bearing mice. FIG. 2H shows expression intensity of Tyr992 pEGFR (left) and identification of the two clusters with highest pEGFR expression (right) projected on t-SNE. FIG. 2I shows MFI of Tyr992 pEGFR in manually-gated immune cell populations. One-way ANOVA, Šídák's multiple comparisons test. j, Density plot of t-SNE from lung tissue stratified by experimental condition; pEGFR⁺ Ly6C⁺ F4/80⁺ population outlined; n=5 per group. FIG. 2K shows IF staining of Ly6C⁺ Tyr992 pEGFR⁺ MNPs in the metastatic TME of LLC lung metastases (Ly6C PE/Dazzle 594 red, Tyr992 pEGFR AF488 green, DAPI blue). FIG. 2L shows frequency of Ly6C⁺ MNPs in the LLC lung metastatic microenvironment post 5, 10 and 20 Gy IR, determined by AI-assisted Cellpose segmentation model and logistic regression classifier. Data are represented as mean±SEM.

FIGS. 3A-3L: scRNA-seq identifies AREG-dependent changes in mononuclear phagocytes in lung tissue from LLC^{AR+} and LLC^{AR-} tumor-bearing mice post-RT. FIG. 3A shows scRNA-seq and unsupervised uniform manifold approximation and projection (UMAP) clustering of 32,690 live CD45⁺ cells isolated from the lungs of LLC^{AR+} and LLC^{AR-} tumor-bearing mice±20 Gy, n=4 mice per group. Outline indicates clusters of mononuclear phagocytes (MNPs). Alv.Mac=alveolar macrophage, conv.=conventional, ILC2=type 2 innate lymphoid cell, NK=natural killer cell, Treg=regulatory T cell. FIG. 3B shows quantification of 5 MNP clusters (outlined in FIG. 4a) stratified by experimental groups. FIG. 3C shows UMAP clustering of 4,095 MNPs in lungs of LLC^{AR+} and LLC^{AR-} tumor-bearing mice±20 Gy, identification of 17 subclusters. Annotation and name assignment using Pegasus auto annotation feature in combination with the most significantly upregulated differentially expressed gene (Mono=monocyte, Alv_macro=alveolar macrophage, P_DC=plasmacytoid DC, Mo_DC=monocyte-derived DC, Int_macro=interstitial macrophage, Migr_DC=migratory DC, C_DC_1=classical type 1 DC). FIG. 3D shows a density plot of 4,095 MNPs in lungs of LLC^{AR+} and LLC^{AR-} tumor-bearing mice±20 Gy stratified by experimental groups. FIG. 3E shows a diffusion map projection of 2,600 monocytes in lungs of LLC^{AR+} and LLC^{AR-} tumor-bearing mice±20 Gy. DC=diffusion component. Cluster color and name assignment consistent with FIG. 3C. FIG. 3F shows a pseudotime diffusion map of 2,600 monocytes. FIG. 3G shows density plots across pseudotime for monocytes from AR+ conditions (LLC^{AR+} and LLC^{AR+}+20 Gy). FIG. 3H shows density plots across pseudotime for monocytes from AR-conditions (LLC^{AR-} and LLC^{AR-}+20 Gy). FIG. 3I shows a slingshot trajectory inference of monocytes projected on diffusion map. DC=diffusion component. FIG. 3J shows the proportion of cells from AR+ and AR-conditions in trajectories 1 and 2. FIG. 3K shows RNA velocity vectors of monocytes projected on diffusion map. DC=diffusion component. FIG. 3L shows a bubble plot showing differential expression of selected genes related to phagocytosis, cell killing, reactive oxygen species (ROS) biosynthetic process, or antigen processing and presentation of trajectories 1 and 2. Signatures based on a previous report³³.

FIGS. 4A-4J: AREG upregulates CD47 in tumor cells resulting in SIRPα-mediated MNP phagocytosis suppression. FIG. 4A shows CD47 MFI of LLC^{AR+} and LLC^{AR-} cells after 48 h treatment with 250 μg/mL rAREG (rAR) or 1.5 μg/mL anti-AREG (αAR). One-way ANOVA, Šídák's multiple comparisons test. FIG. 4B shows immunofluorescence (IF) staining of Ly6C⁺ MNPs showing Ly6C-expression (PE/Dazzle 594, red), Myosin IIA expression (AF488, green) and SIRPα-expression (AF 647, magenta). FIG. 4C shows IF staining of Ly6C⁺ MNPs co-cultured with LLC tumor cells, stained for phosphorylated myo-IIA (AF488, green) and CD47 (BV421, blue). Close-up of phagocytic synapses. FIG. 4D shows IF staining of Ly6C⁺ MNPs co-cultured with LLC tumor cells. Stained for phosphorylated myo-IIA (AF488, green), SIRPα (AF647, magenta) and CD47 (BV421, blue). Bottom right: intensity profile of phospho-myo-IIA and SIRPα measured along white dashed line representing full length of Ly6C⁺ MNP in direct interaction with two tumor cells. Dotted lines represent regions of cell-cell contact. FIG. 4E shows IF staining of Ly6C⁺ MNPs co-cultured with LLC^{AR+} (top row) and LLC^{AR-} (bottom row) tumor cells (left). Stained for phospho-myo-IIA (AF488, green), Ly6C (PE/Dazzle 594, red) and CD47 (BV421, blue). Center: heatmap of phospho-myo-IIA MFI on close-ups of phagocytic synapses. Right: outlines of membrane regions identified as phagocytic synapse (white) and distant non-interactive site (red). FIG. 4F shows the phagocytic ratio of BMDM-derived Ly6C⁺ MNPs co-cultured with LLC^{AR+} and LLC^{AR-} cells. Unpaired t-test. FIG. 4G shows the phagocytic ratio of Ly6C⁺ MNPs isolated from lungs co-cultured with LLC^{AR+} and LLC^{AR-} cells. Unpaired t-test. FIG. 4H shows the IF staining of Ly6C⁺ MNPs isolated from lungs, co-cultured with LLC^{AR+} (top) and LLC^{AR-} (bottom). Stained for phospho-myo-IIA (AF488, green) and CD47 (BV421, blue). FIG. 4I shows the phagocytic ratio of MNPs co-cultured with LLC^{AR-} cells after 48 h treatment with 250 μg/mL rAREG or 1.5 μg/mL anti-AREG. One-way ANOVA, Šídák's multiple comparisons test. FIG. 4J shows the phagocytic ratio of MNPs co-cultured with LLC^{AR-} cells after 48 h treatment with 250 μg/mL rAREG or 1.5 μg/mL anti-AREG. One-way ANOVA, Šídák's multiple comparisons test. Data are presented as mean±SEM or box plots (min to max). One representative experiment out of two independent experiments shown.

FIGS. 5A-5H: AREG-blockade reduces metastasis size in combination with IR, EGFR-TKI, and αCD47 immunotherapy. FIG. 5A shows a tumor growth curve of LLC flank tumors±i.v. AREG-antibody (αAR) and 20 Gy. Two-way ANOVA, Tukey's multiple comparison test. FIG. 5B shows the number of lung metastases per section (left), and mean size of the five largest metastases/lung normalized to lung area (right). One-way ANOVA, Šídák's multiple comparisons test. FIG. 5C shows representative H&E staining of lungs. Black arrows indicate metastatic lesions. FIG. 5D shows a tumor growth curve of LLC flank tumors±i.v. AREG-antibody (αAR), p.o. EGFR-TKI (Gefitinib) and 20 Gy IR. Two-way ANOVA, Tukey's multiple comparison test. FIG. 5E shows the number of lung metastases per section (left), and mean size of the five largest metastases/lung normalized to lung area (right). FIG. 5F shows a representative H&E staining of lungs. Black arrows indicate metastatic lesions. FIG. 5G shows a tumor growth curve of LLC^{AR+} flank tumors±i.v. AREG-antibody (αAR), i.v. CD47-antibody (αCD47) and 20 Gy treatment. Two-way ANOVA, Tukey's multiple comparison test. FIG. 5H shows the number of lung metastases per section (left), and mean size of the five largest metastases/lung normalized to lung area (right). Data are represented as mean±SEM.

FIGS. 6A-6N: Characterization of LLC lung metastasis model reveals dose-dependent AREG secretion from tumor tissue following RT. FIG. 6A shows RT-induced pathways that correlated with distant metastasis progression in patients as assessed by Ingenuity Pathway Analysis. Selection of top 20 upregulated pathways shown; pathways including AREG marked by arrows and labelled red. FIG. 6B shows Kaplan-Meier curves showing the overall survival of patients from The Cancer Genome Atlas (TCGA) undergoing RT and stratified by tumor AREG expression. LUSC=lung squamous cell carcinoma, (Log-rank [Mantel-Cox] test). FIG. 6C shows a schematic of experimental setup in which either LLC flank tumors (1) or a non-tumor bearing hind limb (2) underwent RT. Five days after RT, serum, lung, skin, tumor, and muscle tissues were collected to quantify AREG concentrations by ELISA. FIG. 6D shows AREG protein concentration in tumor (left), serum (center), and lung (right) of LLC tumor-bearing mice five days after RT of the flank tumor, measured by ELISA. One-way ANOVA, Šídák's multiple comparisons test. FIG. 6E shows AREG protein concentration in serum (left), muscle (center), and skin (right) of non-tumor-bearing mice five days after RT of the hind limb, measured by ELISA. One-way ANOVA, Šídák's multiple comparisons test. FIG. 6F shows the tumor growth curve of LLC tumors±20 Gy in Vav1^{ΔAreg} and Areg^{flx/flx} mice, n=5 per group. Two-way ANOVA, Tukey's multiple comparison test. FIG. 6G shows the number (left) of macroscopic metastases and weight of lungs representative of metastatic load (right) in Vav1^{ΔAreg} and Areg^{flx/flx} mice. One-way ANOVA, Šídák's multiple comparisons test. FIG. 6H shows the number of lung metastases (left), and mean size of the five largest metastases/lung normalized to lung area (right) on days 12, 15 and 18 following subcutaneous LLC tumor injection. FIG. 6I shows the representative H&E staining. Black arrows indicate additional metastatic lesions. FIG. 6J shows the number of lung metastases (left), and mean size of the five largest metastases/lung normalized to lung area (right) of day 8 RT group. Unpaired t-test. FIG. 6K shows the number of lung metastases (left), and mean size of the five largest metastases/lung normalized to lung area (right) of day 20 RT group. Unpaired t-test. FIG. 6L shows AREG protein concentration in serum of tumor-bearing mice±20 Gy RT, measured by ELISA. RT timepoints as indicated. One-way ANOVA, Šídák's multiple comparisons test. FIG. 6M shows representative H&E stainings. Black arrows indicate metastatic lesions. FIG. 6N shows a simple linear regression between serum AREG concentration (pg/mL) five days post RT and size of subcutaneous flank tumors on day of RT. Dots are colored as follows: blue=d8 RT, red=d20 RT. (Pearson's correlation coefficient R value and P value shown). Data are represented as mean±SEM or box plots (min to max).

FIGS. 7A-7P: Characterization of spontaneously metastatic orthotopic lung LLC and subcutaneous EO771-LMB models. FIG. 7A shows Areg upregulation in LLC^{AR+} and LLC^{AR-} cells after 4 Gy RT in vitro as determined by RT-qPCR 3 hours after RT. One-way ANOVA, Šídák's multiple comparisons test. FIG. 7B shows AREG protein concentration in flank tumor lysate±20 Gy RT, measured by ELISA 5 days following RT. One-way ANOVA, Šídák's multiple comparisons test. FIG. 7C shows AREG protein concentration in serum of tumor-bearing mice±20 Gy RT, measured by ELISA. Blood draw timepoints post-RT as indicated; ctrl=non-tumor bearing mice. Two-way ANOVA, Tukey's multiple comparison test. FIG. 7D shows representative CT-images of orthotopic LLC^{AR+} and LLC^{AR-} tumors on days 8, 11 and 14 post injection. White asterisk and red outlines indicate orthotopic tumors. FIG. 7E shows tumor growth curves of LLC^{AR+} and LLC^{AR-} orthotopic lung tumors±8 or 12 Gy CT-guided RT. Black arrow indicates timing of RT. Two-way ANOVA, Tukey's multiple comparison test. FIG. 7F shows AREG protein concentration in serum of LLC^{AR+} and LLC^{AR-} orthotopic lung tumor-bearing mice five days after 8 or 12 Gy RT, measured by ELISA. One-way ANOVA, Šídák's multiple comparisons test. FIG. 7G shows the number of contralateral lung metastases (left) and mean size of the five largest metastases/contralateral lung area (right) from LLC^{AR+} and LLC^{AR-} orthotopic tumors, normalized to area. One-way ANOVA, Šídák's multiple comparisons test. FIG. 7H shows the representative H&E stainings of LLC^{AR+} and LLC^{AR-} orthotopic lung tumors. Black arrows indicate contralateral intrapulmonary metastatic lesions, white asterisks indicate orthotopic tumors, LN indicates extrapulmonary, intrathoracic lymph node metastasis. FIG. 7I shows AREG MFI of LMB and LLC tumor cells after 4 and 8 Gy RT, determined by flow cytometry. FIG. 7J shows tumor growth curves of LMB flank tumors±RT. Tumors were irradiated at 150 mm³ with indicated doses, n=5 per group. Two-way ANOVA, Tukey's multiple comparison test. FIG. 7K shows the number of LMB lung metastases (left), size of individual lung metastases normalized to lung area (right). FIG. 7L shows representative H&E staining of LMB tumor-bearing lungs 20 days after RT of flank tumors. Black arrows indicate metastatic lesions. RT doses as indicated. FIG. 7M shows Areg upregulation in LMB^{AR+} and LMB^{AR-} cells after 4 Gy RT in vitro as determined by RT-qPCR post RT. One-way ANOVA, Šídák's multiple comparisons test. FIG. 7N shows tumor growth curves of LMB^{AR+} and LMB^{AR-} flank tumors±5 Gy; n=5 per group. FIG. 7O shows the number of lung metastases (left), and mean size of five largest metastases/lung normalized to lung area (right). FIG. 7P shows the representative H&E staining of lungs. Black arrows indicate metastatic lesions. Two sections at distance of 300 μm analyzed per lung. Data are represented as mean±SEM or box plots (min to max).

FIGS. 8A-8H: Proliferative, migratory and clonogenic behavior of AR+ and AR− LLC and LMB cells with and without RT. FIG. 8A shows In vitro proliferation of tumor cells treated with recombinant AREG. Proliferation measured as percent of confluency, analyzed by live cell imaging over 72 hours. Two-way ANOVA, Tukey's multiple comparison test. FIG. 8B shows in vitro horizontal migration after scratch-wound assay of tumor cells treated with recombinant AREG. Migration measured as percent of confluency of wound area, analyzed by live cell imaging over 48 hours. Two-way ANOVA, Tukey's multiple comparison test. FIG. 8C shows in vitro proliferation of tumor cells treated with RT. Proliferation measured as percent of confluency, analyzed by live cell imaging over 72 hours. Cells were irradiated with 0, 2, 4 or 6 Gy 24 hours before seeding. Two-way ANOVA, Tukey's multiple comparison test. FIG. 8D shows in vitro migration measured by transwell assay. 1×10³ cells were plated in serum-free media in inserts (7 μm pore size) overnight. Cells in the mesh were counted as migrating cells (left), representative images of migrated cells in mesh (right). FIG. 8E shows in vitro migration post RT measured by transwell assay. 1×10³ cells were plated in serum-free media in inserts (7 μm pore size) overnight. Cells were irradiated with 0, 2, 4 or 6 Gy 24 hours before seeding. Two-way ANOVA, Tukey's multiple comparison test. FIG. 8F shows representative images of migrated cells post RT in mesh. FIG. 8G shows in vitro colony formation of tumor cells treated with RT. Cells were irradiated with 0, 2, 4, 6 or 8 Gy 24 h before seeding. Colonies containing >50 cells were counted 10 days after seeding. FIG. 8H shows in vitro colony formation of AR+ and AR-tumor cells treated with recombinant AREG. Colonies containing >50 cells were counted 10 days after seeding. Data are represented as mean±SEM and boxplots (min to max). One representative experiment out of two-three independent experiments shown.

FIGS. 9A-90: Recombinant AREG mimics tumor cell-derived AREG phenotype in vivo. FIG. 9A shows representative H&E staining of lungs 7 days post i.v. injection of 1×10⁵ LLC^{AR+} and LLC^{AR-} tumor cells. Black arrows indicate metastatic lesions. FIG. 9B shows the number of lung metastases per section (left), and mean size of five largest metastases/lung normalized to lung area (right). Unpaired t-test. FIG. 9C shows the representative H&E staining of lungs 14 days post i.v. injection of 1×10⁵ LLC^{AR+} and LLC^{AR-} tumor cells. Black arrows indicate metastatic lesions. FIG. 9D shows the number of lung metastases per section (left), and mean size of five largest metastases/lung normalized to lung area (right). Unpaired t-test. FIG. 9E shows the number of metastases per lung (left) and mean metastasis size after±intravenous (i.v.) recombinant Areg (rAR) treatment normalized to lung area (right). FIG. 9F shows a tumor growth curve of LLC flank tumors±20 Gy RT or complete surgical resection of the flank tumor on day 11, 14 and 18. Two-way ANOVA, Tukey's multiple comparison test. FIG. 9G shows AREG protein concentration in serum of tumor-bearing mice five days after 20 Gy RT or complete surgical resection of the flank tumor on day 11, 14 and 18, measured by ELISA. One-way ANOVA, Šídák's multiple comparisons test. FIG. 9H shows the number of lung metastases (left), and mean size of the five largest metastases/lung normalized to lung area (right) at experimental endpoint—day 28. One-way ANOVA, Šídák's multiple comparisons test. FIG. 9I shows a representative H&E staining. Black arrows indicate additional metastatic lesions. FIG. 9J shows a dot plot of Gene Set Enrichment Analysis (GSEA) comparing upregulated Hallmark pathways in RNA-seq of LLC^{AR+}+4 Gy. FIG. 9K shows a volcano plot of up- and downregulated genes in LLC^{AR+} cells+4 Gy RT. Significantly upregulated DEGs involved in type-I-IFN pathway colored in red. FIG. 9L shows the relative expression of Areg in LLC^{AR+} cells after interferon β treatment, measured by RT-qPCR. One-way ANOVA, Šídák's multiple comparisons test. FIG. 9M shows AREG protein concentration in LLC^{AR+} cells after interferon β treatment, measured by ELISA. One-way ANOVA, Šídák's multiple comparisons test. FIG. 9N shows the relative expression of Areg in LLC cells post-RT and siSTAT2 treatment, measured by RT-qPCR. FIG. 9O shows AREG protein concentration in LLC^{AR+} cells after 4 Gy RT and siSTAT2 treatment, measured by ELISA. One-way ANOVA, Šídák's multiple comparisons test. Data are presented as mean±SEM and box plots (min to max).

FIGS. 10A-10K: Extended information about human PBMC and immune populations in LLC^{AR+} and LLC^{AR-} lungs±RT. FIG. 10A shows spectral flow cytometry and unsupervised t-SNE clustering of 800,000 live CD45⁺ PBMCs of 15 patients pre- and post SBRT. Expression intensity of select PBMC markers. FIG. 10B shows unsupervised cluster assignment of 25 identified PBMC clusters (k-nearest neighbors & FlowSOM). FIG. 10C shows marker expression in heatmap of 25 identified PBMC clusters. Black box highlights CD33⁺ CD14⁺ pEGFR⁺ monocytes (population 4). FIG. 10D shows spectral flow cytometry and unsupervised t-SNE clustering of 230,000 live CD45⁺ immune cells from lungs of non-tumor-bearing and tumor-bearing mice. Expression intensity of select murine immune markers. FIG. 10E shows unsupervised cluster assignment of 16 identified murine lung clusters (k-nearest neighbors & FlowSOM). FIG. 10F shows marker expression in heatmap of 16 identified murine lung clusters. Black box highlights Ly6C⁺, F4/80⁺, pEGFR⁺ monocyte-derived macrophages (population 3). FIG. 10G shows density plots of t-SNE from lung tissue of non-tumor-bearing vs. tumor-bearing mice. Clusters containing myeloid and adaptive immune cells were outlined manually. FIG. 10H shows a heatmap of normalized fold changes of populations of interest identified by surface marker expression in conventional gating. Asterisk marks increase of Ly6C⁺, F4/80⁺ population in LLC^{AR+}+20 Gy condition. FIG. 10I shows the number of CD8⁺ and CD4⁺ T cells and CD19⁺ B cells in the lung tissue of LLC^{AR+} and LLC^{AR-}±20 Gy tumor-bearing mice, identified by surface marker expression in conventional gating, shown as count/mg lung tissue, n=5 mice per group. One-way ANOVA, Šídák's multiple comparisons test. FIG. 10J shows a schematic overview of workflow for AI-based cell segmentation model followed by logistic regression classifier training. FIG. 10K shows a confusion matrix showing the performance of the classification model. All IF stainings: DAPI nuclear stain=blue, Tyr992 pEGFR=green, Ly6C=red. Data are presented as box plots (min to max).

FIGS. 11A-11I: Extended information about immune populations identified by scRNA-seq in LLC^{AR+} and LLC^{AR} lungs±20 Gy. FIG. 11A shows scRNA-seq and unsupervised uniform manifold approximation and projection (UMAP) of 32,690 live CD45⁺ cells in the lungs of LLC^{AR+} and LLC^{AR-} tumor-bearing mice±20 Gy, n=4 mice per group, annotated as 11 major cell lineages. FIG. 11B shows UMAP of live CD45⁺ cells stratified by experimental condition. Monocytes, B cells, T cells, NK cells and neutrophils outlined FIG. 11C shows t-SNE projection of the global transcriptomic profile of 16 individually hashtagged mice from four experimental conditions. FIG. 11D shows a bubble plot showing expression of top three marker genes for 11 major cell lineages. DEGs determined by Wilcoxon rank-sum test.

FIG. 11E shows a volcano plot of up- and downregulated genes in monocytes from LLC^{AR+}+20 Gy. Genes are colored by function. P-values of 0 are depicted at 1-50 (log 10). Wilcoxon rank-sum test. FIG. 11F shows a volcano plot of up- and downregulated genes in monocytes from LLC^{AR-}+20 Gy. Genes are colored by function. Wilcoxon rank-sum test. FIG. 11G shows UMAP of MNPs stratified by experimental condition. Monocytes, conv. type I DCs, plasmacytoid DCs, migratory DCs and alveolar macrophages outlined. Annotation and name assignment using Pegasus auto annotation feature (Mono=monocyte, Alv_macro=alveolar macrophage, P_DC=plasmacytoid DC, Mo_DC=monocyte-derived DC, Int_macro=interstitial macrophage, Migr_DC=migratory DC, C_DC_1=classical type 1 DC) in combination with most significantly upregulated DEG. Cluster color and name assignment consistent with FIG. 3. FIG. 11H shows a dot plot and bar graph representing the fraction of cells within the 17 MNP subclusters stratified by experimental groups. FIG. 11I shows a volcano plot of up- and downregulated genes in population 1 (Mono_Fn1). Genes are colored by function. P-values of 0 are depicted at 1-50 (log 10). Wilcoxon rank-sum test.

FIGS. 12A-12O: Extended information about pseudotime and trajectory analysis of monocytes identified by scRNA-seq. FIG. 12A shows UMAP clustering of 4,095 MNPs identified by scRNA-seq in the lungs of LLC^{AR+} and LLC^{AR-} tumor-bearing mice±20 Gy, n=4 mice per group. Clusters used for pseudotime analysis outlined, 2,600 monocytes. Cluster color and name assignment consistent with FIG. 3c. FIG. 12B shows a heatmap of top marker differentially expressed genes (DEGs) of the 7 populations used for trajectory analysis. FIG. 12C shows a violin plot of pseudotime values of 2,600 monocytes included in the trajectory analysis, categorized by experimental condition. FIG. 12D shows a violin plot of pseudotime values of 7 monocyte subpopulations included in the trajectory analysis. FIG. 12E shows the quantification of cells from four experimental groups in population 1. FIG. 12F shows a heatmap showing differential expression of Reactome pathways related to EGFR signaling in population 1 vs. all other populations. FIG. 12G shows the quantification of cells from four experimental conditions in Pop3 Mono_S100a9 and Pop7 Mono_Adgre4. FIG. 12H shows a volcano plot of significantly enriched genes identified by differential expression analysis in populations 3 and 7. Genes are colored by function. P-values of 0 are depicted at 1-50 (log 10). Wilcoxon rank-sum test. FIG. 12I shows a pie chart showing splicing ratios of total monocyte RNA detected by the scVelo package. FIG. 12J shows a bar plot showing splicing ratios of 7 monocyte populations detected by the scVelo package. FIG. 12K shows gene-level phase portraits depicting un-spliced vs spliced ratio for selected marker genes Thbs1 and Clec4e. Right panels depict inferred velocity of gene and gene expression projected onto diffusion map embedding of monocyte clusters. FIG. 12L shows a time-series heatmap of the top forty velocity-informed putative driver genes visualized across all trajectory monocyte clusters in ascending order of diffusion pseudotime. FIG. 12M shows a volcano plot of significantly enriched genes identified by differential expression analysis in trajectories 1 and 2. Genes are colored by function. P-values of 0 are depicted at 1-50 (log 10). Wilcoxon rank-sum test. FIG. 12N shows expression of select genes across diffusion map projection. FIG. 12O shows a heatmap of Reactome pathway enrichment of trajectories 1 and 2 related to antigen processing and presentation, inflammation and EGFR signaling.

FIGS. 13A-13M: AREG induces T-cell suppressive phenotype in monocytes, but T cells do not determine in vivo lung metastasis phenotype. FIG. 13A shows a heatmap of individual genes and overall gene signature genes involved in immunosuppression in monocyte cluster identified in FIG. 3A. FIG. 13B shows a heatmap of individual genes and overall gene signature of Terminal T cell exhaustion¹ in CD8⁺ T cell 1 cluster identified in FIG. 3a. FIG. 13C shows a schematic of experimental setup. BMDM from Egfr^{flx/flx} (EGFR+) and LysM^{ΔEgfr-/-} (EGFR−) mice were co-cultured overnight with LLC^{AR+} or LLC^{AR-} tumor cells and subjected to fluorescence-activated cell sorting (FACS) of Ly6C⁺ cells, followed by RNA sequencing (RNA-seq). FIG. 13D shows a dot plot of Gene Set Enrichment Analysis (GSEA) comparing upregulated Hallmark pathways in conditions 2 vs. 1 (LLC^{AR-}+EGFR+ MNPs vs. LLC^{AR+}+EGFR+ MNPs). FIG. 13E shows a dot plot of GSEA comparing upregulated Hallmark pathways in conditions 4 vs. 3 (LLC^{AR-}+EGFR− MNPs vs. LLC^{AR+}+EGFR− MNPs). FIG. 13F shows a heatmap of normalized counts of genes involved in cell killing in RNA-seq of Ly6C⁺ MNPs (all gene signatures adapted from a previous report³³). FIG. 13G shows a heatmap of normalized counts of genes involved in ROS biosynthetic processes in RNA-seq of Ly6C⁺ MNPs. FIG. 13H shows a heatmap of normalized counts of genes involved in phagocytosis in RNA-seq of Ly6C⁺ MNPs. FIG. 13I shows a heatmap of normalized counts of genes involved in immunosuppression in RNA-seq of Ly6C⁺ MNPs. FIG. 13J shows the quantification of proliferating T cells after co-culturing with bone-marrow derived monocytes (BMDM) (top) and histogram of proliferating CD8⁺ T cell populations (bottom). FIG. 13K shows a tumor growth curve of LLC^{AR+} and LLC^{AR-}±i.v. CD8-antibody (αCD8). n=5 per group. Two-way ANOVA, Tukey's multiple comparison test. FIG. 13L shows the number of metastases (left), and mean metastasis area after αCD8 treatment, normalized to lung area (right). Two sections per lung analyzed at a distance of 300 μm. One-way ANOVA, Šídák's multiple comparisons test. FIG. 13M shows a representative H&E staining of lungs at experimental endpoints. Black arrows indicate metastatic lesions. Data are presented as mean±SEM and box plots (min to max).

FIGS. 14A-14E: Anti-CCR2 antibody-mediated depletion of MNPs determines in vivo lung metastasis phenotype. FIG. 14A shows the number of CD11b⁺ Ly6C⁺ monocytes, CD11b⁺ F4/80⁺ macrophages, CD11c⁺ MHCII⁺ dendritic cells, CD11b⁺ Ly6G⁺ granulocytes and CD11b⁻ SiglecF⁺ alveolar macrophages per μg lung tissue, isolated from lungs of LLC^{AR+} tumor-bearing mice treated with anti-CCR2 antibody (αCCR2). n=5 per group. Populations identified using conventional gating strategy. Unpaired t test. FIG. 14B shows the number of lung metastases (left), and mean size of the five largest metastases/lung normalized to lung area (right) of LLC^{AR+}+αCCR2 at experimental endpoint. Unpaired t test. FIG. 14C shows representative H&E stainings. Black arrows indicate additional metastatic lesions. FIG. 14D shows the number of lung metastases (left), and mean size of the five largest metastases/lung normalized to lung area (right) of LLC^{AR-}+αCCR2 at experimental endpoint. Unpaired t test. FIG. 14E shows representative H&E stainings. Black arrows indicate additional metastatic lesions. Data are presented as box plots (min to max).

FIGS. 15A-15I: AREG upregulates CD47 expression via STAT3 signaling to suppress phagocytosis of monocytic phagocytes. FIG. 15A shows a schematic of live cell imaging co-culture system with CellTracker Red positive tumor cells and CellTracker Green positive BMDM (top). Fraction of engulfed LLC^{AR+} and LLC^{AR-} tumor cells after 12 hours (bottom). FIG. 15B shows representative images of live cell imaging co-culture system with CellTracker Red positive tumor cells and CellTracker Green positive BMDM. FIG. 15C shows expression of Cd47 in LLC^{AR+} (left) and LLC^{AR-} cells post-RT (right), measured by RT-qPCR. One-way ANOVA, Šídák's multiple comparisons test. FIG. 15D shows a histogram and quantification of CD47 expression on LLC^{AR+} and LLC^{AR-} tumor cells±4 Gy in vitro. FIG. 15E shows the mean fluorescence intensity (MFI) of pSTAT3 (Tyr705) expression, gated on LLC cells, after 25 minutes of rAREG treatment with indicated concentrations. FIG. 15F shows expression of Cd47 in LLC^{AR+} cells post-rAR treatment, measured by RT-qPCR. One-way ANOVA, Šídák's multiple comparisons test. FIG. 15G shows expression of Cd47 in LLC^{AR+} cells post-RT, rAR and STAT3i (Stattic) treatment, measured by RT-qPCR. One-way ANOVA, Šídák's multiple comparisons test. FIG. 15H shows IF staining of Ly6C⁺ MNPs co-cultured with LLC tumor cells. Stained for unphosphorylated myo-IIA (AF488, green) and CD47 (BV421, blue). Close-up of phagocytic synapse. FIG. 15I shows IF staining of Ly6C⁺ MNPs co-cultured with LLC^{AR+} (top row) and LLC^{AR-} (bottom row). Stained for phospho-myo-IIA (AF488, green), Ly6C (PE/Dazzle 594, red) and CD47 (BV421, blue). Data are presented as mean±SEM.

FIGS. 16A-16C: AREG blockade synergizes with anti-CD47 and increases MNP phagocytosis in vivo. FIG. 16A shows a Kaplan-Meier plot showing progression-free survival stratified by AREG serum levels as measured by ELISA. n=42 samples from n=21 patients pre-SBRT and after three cycles of ICB+SBRT. (Log-rank [Mantel-Cox] test). FIG. 16B shows exemplary gating strategy for murine tissue identifying RFP+ phagocytic MNP populations. FIG. 16C shows the percentage of phagocytic RFP⁺ Ly6C⁺ monocytes (left), RFP⁺ Ly6G⁺ neutrophil granulocytes, RFP+CD11c⁺ MHCII⁺ dendritic cells and RFP⁺ CD11b⁻ Siglec-F⁺ alveolar macrophages shown as percentage of parent population. Cells isolated from lungs of LLC^{AR+} tumor-bearing mice treated with 20 Gy RT, anti-AREG−, anti-CD47-antibody or a combination of all. n=5-6 per group. One-way ANOVA, Šídák's multiple comparisons test. Data are presented as box plots (min to max).

FIGS. 17A-17I: Total immune phenotype and Tyr992 pEGFR expression on Ly6C⁺ MNPs from lung tissue of LLC^{AR+} tumor-bearing mice treated with 20 Gy RT, αAR and αCD47. FIG. 17A shows the percentage of Ly6C⁺ monocytes, CD11b⁻ SiglecF⁺ alveolar macrophages and CD3⁺ CD8⁺ T cells, shown as percentage of live CD45⁺ immune cells isolated from lungs of LLC^{AR+} tumors-bearing mice treated with 20 Gy RT, αAR and αCD47. n=5 per group. Populations identified using conventional gating strategy. One-way ANOVA, Šídák's multiple comparisons test. FIG. 17B a t-SNE dimensionality reduction of 250,000 live CD45⁺ immune cells from lung tissue. FIG. 17C expression intensity of population-defining extracellular markers projected on t-SNE. FIG. 17D an identification of 16 populations by unsupervised clustering (k-nearest neighbors & FlowSOM). FIG. 17E the expression intensity of Tyr992 pEGFR projected on t-SNE. FIG. 17F the identification of two clusters with highest pEGFR expression values (Clusters 13 and 15). FIG. 17G a heatmap showing marker expression of 16 clusters. Black boxes highlight populations with highest expression values of Tyr992 pEGFR (populations 13 and 15). FIG. 17H a density plot of t-SNE of live CD45⁺ cells from lungs stratified by experimental condition; pEGFR⁺ MNP populations outlined. FIG. 17I the percentage of pEGFR⁺ MNP populations of live CD45⁺ immune cells. One-way ANOVA, Šídák's multiple comparisons test. Data are presented as box plots (min to max).

FIGS. 18A-18E: Amphiregulin (AREG) and association with survival, response, and baseline features in COSINR. FIG. 18A shows a forest plot of overall survival and pre-treatment, post-treatment, and pre-minus post-treatment AREG expression. FIG. 18B shows the correlation between baseline AREG and baseline tumor burden measured by sum of RECIST measurements. FIG. 18C shows the correlation between change in AREG and change in sum of RECIST measurements. FIG. 18D shows OS in AREG-high patients stratified by treatment of all RECIST eligible lesions. FIG. 18E shows the log 2-normalized expression of AREG protein in patients with PD-L1-high tumors (a tumor proportion score ≥50%) compared to PD-L1-low patients during pre-treatment (left) or late-treatment (center); the right plots show late-treatment minus pre-treatment.

FIGS. 19A-19E: Peripheral proteomic, flow cytometry, and TCR correlates in COSINR. FIG. 19A shows the ssGSEA of peripheral blood proteomic findings using Hallmark and immune-related reactome pathways calculated at pre-treatment, post-treatment, and change pre- to post-treatment. FIG. 19B shows a volcano plot of individual protein expression correlated to post-treatment AREG expression at pre- and post-treatment. P-value cutoff p<0.001, expression change cutoff 0.3. FIGS. 19C-D show the correlation of post-treatment AREG with immune cell populations in peripheral blood measured pre-treatment, post-SBRT and pre-ipi/nivo, and post-treatment with both SBRT and ipi/nivo. Individual time points are shown in FIG. 19C, while FIG. 19D shows change between time points. FIG. 19E shows the correlation of T-cell receptor sequencing findings with post-treatment AREG. All correlations calculated using Spearman's rank order calculation.

FIG. 20 depicts a graphical abstract of the biology underlying the interactions between AREG, EGFR, STAT3, CD47, and their effects on tumor progression and metastasis.

### DETAILED DESCRIPTION

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the meaning commonly understood by one of ordinary skill in the art. Unless otherwise required by context, singular terms shall include the plural and plural terms shall include the singular.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

In some embodiments, percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

The use of the alternative (e.g., “or”) should be understood to mean either one, both, or any combination thereof of the alternatives unless otherwise indicated.

In the present disclosure, any concentration range, percentage range, ratio range, or integer range is to be understood to include the value of any integer within the recited range and, when appropriate, fractions thereof (such as one tenth and one hundredth of an integer), unless otherwise indicated.

Unless expressly specified otherwise, the term “comprising” is used in the context of the present disclosure to indicate that further members may optionally be present in addition to the members of the list introduced by “comprising.”

### Overview

Radiotherapy (RT) is primarily used to treat localized tumors, but there is increasing interest in its use as a potentially curative treatment of oligometastatic disease or in patients with widespread metastases in combination with immune checkpoint blockade (ICB). The epidermal growth factor receptor (EGFR) is activated by amphiregulin (AREG) through dimerization and autophosphorylation, and is an integral part of type 2 immune-mediated tolerance and resistance mechanisms in various immune cells. AREG overexpression has been found in a wide variety of human cancers and has been associated with intestinal recovery following RT in preclinical models.

We identified RT-dependent induction of AREG in patients with metastatic solid tumors enrolled in a clinical trial of multisite stereotactic body radiotherapy (SBRT) (NCT02608385). RT-mediated AREG induction was associated with distant metastasis progression. In murine models of lung metastasis, tumor cell-derived AREG enabled lung metastasis progression by reprogramming EGFR+ mononuclear phagocytes (MNPs) towards an immunosuppressive/anti-inflammatory phenotype. AREG reduced MNP phagocytosis via increased CD47 expression on tumor cells. Importantly, targeting AREG suppressed RT-induced metastasis growth and enhanced anti-tumor efficacy of IR.

While most of the “classical” predictors of RT outcome, such as tumor histology, size, and molecular subtype are not amenable to modification following the initiation of RT, inhibiting specific radiation-induced metastasis-promoting proteins may improve RT efficacy. EGFR blockade has previously been tested in clinical trials. However, blocking RT-induced upregulation of AREG in radio- or immuno-therapy is new.

In some embodiments, the present disclosure provides a composition for treating metastatic cancer in a subject in need thereof, the composition including: a) a therapeutically effective amount of an AREG inhibitor; and b) a therapeutically effective amount of a radiotherapy agent.

As used herein, the term “subject” or “patient” refers to a human or non-human animal to whom a composition, formulation, or method described herein is administered, or in whom a disease, disorder, or condition is diagnosed, prognosed, monitored, treated, or prevented. The term encompasses mammals such as humans, non-human primates, rodents, canines, felines, equines, bovines, ovines, porcines, and other veterinary or laboratory animals. Unless otherwise specified, the terms are used interchangeably and without limitation as to age, sex, or health status.

As used herein, the term “therapeutically effective amount” refers to a quantitative or qualitative value, range, or threshold of a biomarker, physiological parameter, or other measurable characteristic, with which a subject's corresponding value is compared for purposes of treatment or therapy. A therapeutically effective amount may be established from one or more control populations (e.g., healthy subjects, subjects with a known disease state, or subjects with a defined clinical outcome), from historical data, or from an earlier measurement obtained from the same subject. A subject's measured level may be deemed elevated, reduced, or within the therapeutically effective amount, and such comparison may inform the approach to treatment and/or therapy. Different reference levels of the same biomarker, physiological parameter, or other measurable characteristic, can be used to diagnose, prognose, or monitor different diseases, disorders, or conditions.

As used herein, the term “reference level” refers to a quantitative or qualitative value, range, or threshold of a biomarker, physiological parameter, or other measurable characteristic, with which a subject's corresponding value is compared for purposes of diagnosis, prognosis, and/or monitoring. A reference level may be established from one or more control populations (e.g., healthy subjects, subjects with a known disease state, or subjects with a defined clinical outcome), from historical data, or from an earlier measurement obtained from the same subject. A subject's measured level may be deemed elevated, reduced, or within the reference level (e.g., physiologically comparable or equivalent), and such comparison may inform the likelihood, presence, absence, stage, severity, or predicted outcome of a disease or condition. Further, a subject's measured level of a particular biomarker when deemed to differ from a reference level may inform decisions regarding treatment for a particular disease or condition associated with such biomarker. Different reference levels of the same biomarker, physiological parameter, or other measurable characteristic, can be used to diagnose, prognose, or monitor different diseases, disorders, or conditions.

As used herein, “radiotherapy” or RT, also known as radiation therapy, is a treatment for cancer and/or tumor growth that uses radiation to shrink or destroy the cancer and/or tumor. RT is effective by targeting, damaging, and destroying DNA so that the cancer cells cannot divide and thus die out. In some embodiments, RT may be provided as external beam radiation therapy, in which the source of the radiation is an external machine that focuses radiation to targeted spots in a patient. In some embodiments, RT may be provided as internal radiation therapy, in which the radiation source is placed within (internally to) the patient. Further embodiments of RT contemplated include: radioimmunotherapy, in which the radiation agent is tethered or linked to an immunological composition such as an antibody or antibody fragment; systemic radiation therapy, a form of internal RT in which a liquid radiation source is provided orally or intravenously; or brachytherapy, in which a solid source is provided.

As used herein, “radiation agent” or (RT agent) refers to a radiologically active atom used in targeted RT. Any radiologically active atom that is effective for treating a tumor is contemplated herein. Examples of radiation agents include, but are not limited to: yttrium-90, iodine-131, samarium-153, lutetium-177, astatine-211, lead-212 with bismuth-212, radium-223, actinium-225, and thorium-227. In some embodiments, where an RT agent is employed, other forms of RT can be used in lieu of or in addition to the RT agent.

As used herein, “inhibitor” refers to a molecule that interacts with at least one target and prevents the target or targets from performing a function. Non-limiting examples of inhibitors include: antibodies, including IgG, IgM, IgE, IgA, and IgD species; antibody-drug conjugates (ADCs); antibody fragments (e.g., antigen-binding fragments), including variable heavy (VH) domains, variable light (VL) domains, single chain fragment variable constructs (scFvs), bispecific T cell engagers (BiTEs), nanobodies, diabodies, and triabodies; protein binders; peptides; RNA including siRNA; and small molecules.

In some embodiments, an AREG inhibitor, an EGFR inhibitors, a CD47 inhibitor, a STAT3 inhibitor, a SIRPα inhibitor, and/or a TACE inhibitor can employed, individually or in varied combinations of two or more, in compositions and methods of the present disclosure, as described below.

In some embodiments, the AREG inhibitor may be one or more of an anti-AREG antibody or an antigen-binding fragment thereof, an anti-AREG antibody drug conjugate (ADC), a protein binder, a peptide, an RNA, an AREG siRNA, a small molecule, and heparin. Any embodiment disclosed or contemplated herein that includes an AREG inhibitor can include any of the disclosed AREG inhibitors.

In some embodiments, the RT agent may be one or more of yttrium-90, iodine-131, samarium-153, lutetium-177, astatine-211, lead-212 with bismuth-212, radium-223, actinium-225, and thorium-227. Any embodiment disclosed or contemplated herein that includes an RT agent can include any of the disclosed RT agents.

In another embodiment of the present disclosure, a composition is provided for treating metastatic cancer in a subject in need thereof, the composition including: a) a therapeutically effective amount of an AREG inhibitor; b) a therapeutically effective amount of a radiotherapy agent; and c) a therapeutically effective amount of an epidermal growth factor receptor (EGFR) inhibitor.

In some embodiments, the EGFR inhibitor may be one or more of an anti-EGFR antibody or an antigen-binding fragment thereof, an anti-EGFR ADC, a protein binder, a peptide, an RNA, an EGFR siRNA, and a small molecule. In some embodiments, the EGFR inhibitor may be a small molecule tyrosine kinase inhibitor (TKI) of the tyrosine kinase domain of EGFR (EGFR TKI). In some embodiments, the EGFR inhibitor may be the anti-EGFR antibody gefitinib. Any embodiment disclosed or contemplated herein that includes an EGFR inhibitor can include any of the disclosed EGFR inhibitors.

In another embodiment of the present disclosure, a composition is provided for treating metastatic cancer in a subject in need thereof, the composition including: a) a therapeutically effective amount of an AREG inhibitor; b) a therapeutically effective amount of a radiotherapy agent; and c) a therapeutically effective amount of a Cluster of Differentiation 47 (CD47) inhibitor.

In some embodiments, the CD47 inhibitor may be one or more of an anti-CD47 antibody or an antigen-binding fragment thereof, an anti-CD47 ADC, a protein binder, a peptide, an RNA, a CD47 siRNA, and a small molecule. Any embodiment disclosed or contemplated herein that includes a CD47 inhibitor can include any of the disclosed CD47 inhibitors.

In another embodiment of the present disclosure, a composition is provided for treating metastatic cancer in a subject in need thereof, the composition including: a) a therapeutically effective amount of an AREG inhibitor; b) a therapeutically effective amount of a radiotherapy agent; and c) a therapeutically effective amount of a Signal transducer and activator of transcription 3 (STAT3) inhibitor.

In some embodiments, the STAT3 inhibitor may be one or more of an anti-STAT3 antibody or an antigen-binding fragment thereof, an anti-STAT3 ADC, a protein binder, a peptide, an RNA, a STAT3 siRNA, and a small molecule. Any embodiment disclosed or contemplated herein that includes a STAT3 inhibitor can include any of the disclosed STAT3 inhibitors.

In another embodiment of the present disclosure, a composition is provided for treating metastatic cancer in a subject in need thereof, the composition including: a) a therapeutically effective amount of an AREG inhibitor; b) a therapeutically effective amount of a radiotherapy agent; and c) a therapeutically effective amount of a Signal regulatory protein α (SIRPα) inhibitor.

In some embodiments, the SIRPα inhibitor may be one or more of an anti-SIRPα antibody or an antigen-binding fragment thereof, an anti-SIRPα ADC, a protein binder, a peptide, an RNA, a SIRPα siRNA, and a small molecule. Any embodiment disclosed or contemplated herein that includes a SIRPα inhibitor can include any of the disclosed SIRPα inhibitors.

In another embodiment of the present disclosure, a composition is provided for treating metastatic cancer in a subject in need thereof, the composition including: a) a therapeutically effective amount of a tumor necrosis factor α converting enzyme (TACE) inhibitor; and b) a therapeutically effective amount of a radiotherapy agent.

In some embodiments, the TACE inhibitor may be one or more of an anti-TACE antibody or an antigen-binding fragment thereof, an anti-TACE ADC, a protein binder, a peptide, an RNA, a TACE siRNA, and a small molecule. Any embodiment disclosed or contemplated herein that includes a TACE inhibitor can include any of the disclosed TACE inhibitors.

In another embodiment of the present disclosure, a composition is provided for treating metastatic cancer in a subject in need thereof, the composition including: a) a therapeutically effective amount of a tumor necrosis factor α converting enzyme (TACE) inhibitor; b) a therapeutically effective amount of a radiotherapy agent; and c) a therapeutically effective amount of an AREG inhibitor.

In another embodiment of the present disclosure, a composition is provided for treating metastatic cancer in a subject in need thereof, the composition including: a) a therapeutically effective amount of an AREG inhibitor; and b) a therapeutically effective amount of an immune checkpoint inhibitor (ICI).

As used herein, an “immune checkpoint inhibitor” is a molecule that binds to proteins involved in the immune system. Often these molecules are inhibitors. Non-limiting examples of ICI targets include: programmed cell death protein 1 (PD-1), programmed death-ligand 1 (PD-L1), and cytotoxic T-lymphocyte associated protein 4 (CTLA-4).

In some embodiments, the ICI may be an inhibitor of PD-1, PD-L1, CTLA-4, or CD47. In some embodiments, the ICI may be one or more of: an α-PD-1 antibody such as camrelizumab, cemiplimab, cetrelimab, nivolumab, pembrolizumab, penpulimab, pidilizumab, retifanlimab, sintilimab, spartalizumab, and/or sugemalimab; an α-PD-L1 antibody such as atezolizumab, avelumab, and/or durvalumab; and/or ipilimumab (α-CTLA-4).

In another embodiment of the present disclosure, a composition is provided for treating metastatic cancer in a subject in need thereof, the composition including: a) a therapeutically effective amount of an AREG inhibitor; and b) a therapeutically effective amount of an EGFR inhibitor.

Various dosages are contemplated for the inhibitors described herein. Inhibitors such as inhibitors to AREG, EGFR, CD47, STAT3, SIRPα, TACE, PD-1, PD-L1, CLTA-4 may be provided at dosages including, but not limited to: 0.001 μg/kg-100 mg/kg body weight. Inhibitors may be provided intravenously, intra-arterially, subcutaneously, intramuscularly, intranasally, orally, or via any other known techniques in the art. Contemplated dosing regiments may also include single deliveries, or repeated deliveries (for example: 2, 3, 4, or more administrations).

Inhibitors may be provided individually or in combination with other inhibitors or anti-tumor therapeutics, such as but not limited to those described herein. Any of the disclosed inhibitors or therapies are contemplated in combination with any of the other disclosed inhibitors or therapies as described herein. In one non-limiting example, inhibitors to AREG may be combined with ICIs, inhibitors to CD-47, inhibitors to EGFR, and RT treatment.

In another embodiment of the present disclosure, a method is provided for treating a tumor (e.g., a primary or metastatic tumor) in a patient, including the steps of:
a) obtaining a sample from the patient;
b) performing an assay on the sample to determine a level of AREG in the sample;
c) determining that the AREG level is higher than a reference level; and
d) administering a therapeutically effective amount of an AREG inhibitor to the patient and/or administering a therapeutically effective amount of a radiotherapy agent to the patient,
whereby the tumor is treated.

In some embodiments, the sample may be one or more of: blood, plasma, and/or tissue. In some embodiments, the sample may be plasma.

In another embodiment of the present disclosure, a method is provided for treating a tumor in a patient, including the steps of:
a) obtaining a sample from the patient;
b) performing an assay on the sample to determine a level of AREG in the sample;
c) determining that the AREG level is higher than a reference level; and
d) administering a therapeutically effective amount of an AREG inhibitor to the patient and administering a therapeutically effective amount of a radiotherapy agent to the patient,
whereby the tumor is treated.

In another embodiment of the present disclosure, a method is provided for treating a tumor in a patient, including the steps of:
a) obtaining a sample from the patient;
b) performing an assay on the sample to determine a level of AREG in the sample;
c) determining that the AREG level is higher than a reference level; and
d) administering a therapeutically effective amount of an AREG inhibitor to the patient and/or administering a therapeutically effective amount of a radiotherapy agent to the patient,
e) performing an assay on the sample to determine a level of circulating plasma EGFR in the sample;
f) determining that the EGFR level is higher than a reference level; and
g) administering a therapeutically effective amount of an EGFR inhibitor to the patient.
whereby the tumor is treated.

In another embodiment of the present disclosure, a method is provided for treating a tumor in a patient, including the steps of:
a) obtaining a sample from the patient;
b) performing an assay on the sample to determine a level of AREG in the sample;
c) determining that the AREG level is higher than a reference level; and
d) administering a therapeutically effective amount of an AREG inhibitor to the patient and/or administering a therapeutically effective amount of a radiotherapy agent to the patient,
e) performing an assay on the sample to determine a level of circulating plasma CD47 in the sample;
f) determining that the CD47 level is higher than a reference level; and
g) administering a therapeutically effective amount of a CD47 inhibitor to the patient.
whereby the tumor is treated.

In another embodiment of the present disclosure, a method is provided for treating a tumor in a patient, including the steps of:
a) obtaining a sample from the patient;
b) performing an assay on the sample to determine a level of AREG in the sample;
c) determining that the AREG level is higher than a reference level; and
d) administering a therapeutically effective amount of an AREG inhibitor to the patient;
and
e) administering a therapeutically effective amount of an ICI to the patient,
whereby the tumor is treated.

In another embodiment of the present disclosure, a method is provided for treating a tumor in a patient, including the steps of:
a) obtaining a sample from the patient;
b) performing an assay on the sample to determine the level of circulating plasma AREG in a subject;
c) determining that the AREG level is higher than a reference level;
d) performing an assay on the sample to determine the level of circulating plasma EGFR in a subject;
e) determining that the EGFR level is higher than a reference level;
f) administering a therapeutically effective amount of an AREG inhibitor to the patient; and
g) administering a therapeutically effective amount of an EGFR inhibitor to the patient, whereby the cancer is treated.

In another embodiment of the present disclosure, a method is provided for treating a tumor in a patient, including the steps of:
determining if an AREG level in the patient is higher than a reference level by:
   a) obtaining a sample from the patient;
   b) performing an assay on the sample to determine the level of circulating plasma AREG in the patient;
if the sample has an AREG level higher than the reference level, then administering a therapeutically effective amount of an AREG inhibitor and a therapeutically effective amount of a radiotherapy agent to the patient, or
if sample has an AREG level lower than the reference level, then administering a therapeutically effective amount of a radiotherapy agent to the patient.

In another embodiment of the present disclosure, a method is provided for treating a tumor in a patient, including the steps of:
determining if an AREG level in the patient is higher than a reference level by:
   a) obtaining a sample from the patient;
   b) performing an assay on the sample to determine the level of circulating plasma AREG in the patient;
   c) determining if an EGFR level is higher than a reference level by performing an assay on the sample to determine the level of circulating plasma EGFR in the patient;
if the sample has an EGFR level higher than the reference level, then administering a therapeutically effective amount of an EGFR inhibitor to the patient, and;
if the sample has an AREG level higher than the reference level, then administering a therapeutically effective amount of an AREG inhibitor and a therapeutically effective amount of a radiotherapy agent to the patient, or
if sample has an AREG level lower than the reference level, then administering a therapeutically effective amount of a radiotherapy agent to the patient.

In another embodiment of the present disclosure, a method is provided for treating a tumor in a patient, including the steps of:
determining if an AREG level in the patient is higher than a reference level by:
   a) obtaining a sample from the patient;
   b) performing an assay on the sample to determine the level of circulating plasma AREG in the patient;
   c) determining if a CD47 level is higher than a reference level by performing an assay on the sample to determine the level of circulating plasma CD47 in the patient;
if the sample has a CD47 level higher than the reference level, then administering a therapeutically effective amount of a CD47 inhibitor to the patient, and;
if the sample has an AREG level higher than the reference level, then administering a therapeutically effective amount of an AREG inhibitor and a therapeutically effective amount of a radiotherapy agent to the patient, or
if sample has an AREG level lower than the reference level, then administering a therapeutically effective amount of a radiotherapy agent to the patient.

All combination of steps are contemplated from the methods described herein, performed in any order. In one non-limiting example, methods may include the steps of a) obtaining a sample from the patient; b) performing an assay to determine the level of at least any one of the following markers: AREG, CD47, CTLA-4, EGFR, PD-1, PD-L1, STAT3, SIRPα, and (or) TACE; and c) determining if the level of at least any one of the following markers: AREG, CD47, CTLA-4, EGFR, PD-1, PD-L1, STAT3, SIRPα, and (or) TACE is above a reference level; and (or) d) administering a therapeutically effective amount of an inhibitor to any of the aforementioned markers.

In another embodiment of the present disclosure, a kit is provided which includes:
a) a first assay for determining a concentration of AREG in a sample; and
b) a second assay for determining a concentration of EGFR in the sample; and/or
c) a third assay for determining a concentration of CD47 in the sample.

In some embodiments, the assays present in the kit may comprise one or more of: an enzyme-linked immunosorbent assay (ELISA); a sandwich immunoassay with an electrochemiluminescence, a bead-based immunoassay, a proximity extension assay (PEA); a colorimetric assay; an immunoassay; an enzyme assay; an ultraviolet (UV) absorbance assay; a chromatography assay such as liquid chromatography or gas chromatography; a cell-based assay; and/or a protein quantification assay such as Bradford or bicinchoninic acid (BCA) assay.

In some embodiments, the sandwich assay may be performed using a Meso Scale Discovery electrochemiluminescence assay. In some embodiments, the bead-based immunoassay may be performed using Luminex xMAP® technology. In some embodiments, the PEA may be performed using the Olink® Target series multiplex panels for PEA.

In some embodiments, the kit may provide methods for preparing samples for assays; non-limiting examples of downstream assays may include fluorescence-activated cell sorting (FACS), mass spectrometry (MS), Western blotting; and co-immunoprecipitation followed by qualitative or quantitative analyses.

Variations of kits are contemplated that include any number of the assays as described herein. In a non-limiting example, a kit may comprise ELISAs for detecting AREG concentration with cell assays for detecting EGFR and (or) CD47 concentration.

### EXAMPLES

The Examples that follow are further illustrations of specific embodiments of the disclosure, and various uses thereof. They are set forth for explanatory purposes only and should not be construed as limiting the scope of the disclosure in any way.

### Example 1: Radiation-Induced Amphiregulin Drives Tumor Metastasis

### Summary

The anti-tumor effect of radiotherapy (RT) beyond the treatment field—the abscopal effect—has garnered much interest. By contrast, the potentially harmful impact of radiation in promoting metastasis is less well studied. Here, we show that RT induces the expression of epidermal growth factor receptor (EGFR) ligand amphiregulin (AREG) in tumor cells, which reprograms EGFR⁺ myeloid cells toward an immunosuppressive phenotype and reduces phagocytosis. This stimulates distant metastasis growth in both patients and pre-clinical murine tumor models. The inhibition of these tumor-promoting factors induced by RT may represent a novel therapeutic strategy to improve patient outcomes.

### Introduction

Here, we identify RT-dependent induction of AREG in patients with metastatic solid tumors enrolled in a clinical trial of multisite stereotactic body radiotherapy (SBRT) (NCT02608385) 21,22. RT-mediated AREG induction was associated with distant metastasis progression. In murine models of lung metastasis, tumor cell-derived AREG enabled lung metastasis progression by reprogramming EGFR+ mononuclear phagocytes (MNPs) 23 towards an immunosuppressive/anti-inflammatory phenotype. AREG reduced MNP phagocytosis via increased CD47 expression on tumor cells. Importantly, targeting AREG suppressed RT-induced metastasis growth and enhanced anti-tumor efficacy of IR. While most of the “classical” predictors of RT outcome, such as tumor histology, size, and molecular subtype are not amenable to modification following the initiation of RT, these results suggest that inhibiting specific radiation-induced metastasis-promoting proteins may improve cancer therapy.

### Methods

### Patient Selection and Sample Collection

Tissue biopsy microarray data: Patients with advanced solid tumors were enrolled between January 2016 and March 2017 (clinicaltrials.gov: NCT0260838²¹). Patients received SBRT to at least two measurable metastases with each receiving 30-50 Gy over 3-5 fractions. Twenty-two matched tumor biopsies were collected, prior to and within 7 days following SBRT. RNA was extracted and analyzed using Affymetrix Human Clariom™ D microarrays. Computational gene expression deconvolution methods were used for genome-wide expression analyses following SBRT and correlated with irradiated tumor response, as scored by RECIST⁶⁷.

### Detection of Genes Upregulated by Irradiation

R package ISOpure® was used to separate treatment-specific expression changes from patient- and histology-specific expression patterns. Genes with an absolute delta value change ≥1 between pre- and post-SBRT were retained for Ingenuity Pathway Analysis (IPA). Canonical pathways and predicted upstream regulator analyses were performed in IPA. Fisher's exact test P-values determined statistical significance. The ratio of post- and pre-SBRT gene expression values was determined for each patient. A 95% confidence interval was calculated for each gene across patients, and those genes having confidence intervals that did not cross zero were retained for subsequent analyses.

PBMC analysis: Patients enrolled in the COSINR study (NCT03223155²⁹) were used for PBMC analysis. The COSINR study is a randomized phase I/II trial designed to evaluate the safety and efficacy of combination ICB using Ipilimumab/Nivolumab plus sequential or concurrent SBRT as a first-line treatment for patients with stage IV NSCLC. Without consideration of PD-L1 expression or tumor mutational burden (TMB), patients were randomized to SBRT to two to four metastatic sites with concurrent or sequential (within 7 days) immunotherapy. PBMCs were obtained prior to treatment and following completion of SBRT (sequential arm).

### Mice

All mice were maintained under specific pathogen-free (SPF) conditions and used in accordance with the animal experimental guidelines set by the Institute of Animal Care and Use Committee (IACUC). This study has been approved by the Institutional Animal Care and Use Committee of the University of Chicago (ACUP no. 70931 and 72213).

Mice used in this work were on a C57BL/6 background. Mice were purchased from Harlan Envigo. Vav1^{□Areg} and Areg^{flx/flx} transgenic mice were purchased from Jackson Laboratory. To generate conditional Egfr-KO mice, LysM-Cre (B6.129P2-Lyz2^{tml(cre)Ifo}/J) were bred with Egfr-floxed mice (Egfr^{flx/flx})⁶⁸, kindly provided by Dr. Douglas Tilley. For tumor studies, 8-10-week old female C57BL/6 (WT) mice were used. For studies using knockout or conditional knockout mice, mice were used at 6 to 10 weeks of age and sex-matched in each experiment. A 12-hour light/dark cycle was used and temperatures of 19-22° C. with 30-70% humidity were maintained.

### Cell Lines and In Vivo Reagents

Cell lines: LLC (CRL-1642) and E0771-LMB (CRL-3405) cells were purchased from ATCC (Manassas, VA) and were maintained according to the method of characterization used by ATCC. Cell lines were not independently authenticated beyond the identity provided from the ATCC. To enhance metastatic homing, both cell lines were passaged twice from spontaneous lung metastatic lesions. CRISPR-Cas9 mediated Areg-depletion was conducted using amphiregulin CRISPR KO plasmid from Santa Cruz (sc-419185). Cells were selected with 5 μg/ml of puromycin (InvivoGen, San Diego, CA), sorted according to RFP-expression and a single cell culture was generated. The knockout cell lines were authenticated by RT-qPCR and flow cytometry. Tumor cells were cultured in 5% CO₂ and were maintained in DMEM medium (Corning, Manassas, VA), supplemented with 10% heat-inactivated fetal bovine serum (Sigma, St Louis, MO), 100 U/ml penicillin, and 100 μg/ml streptomycin.

In vivo reagents: Mouse Amphiregulin Biotinylated Antibody (BAF989) and recombinant mouse amphiregulin protein (989-AR-100)—R&D systems. Gefitinib (Iressa®, ZD1839)—Selleck Chem. InVivoMAb anti-mouse CD47 (IAP, #BE0270)—BioXcell.

### Tumor Growth, Murine RT, CT-Imaging and In Vivo Treatments

1×10⁶ tumor cells were subcutaneously (s.c.) injected in the flanks of 8 to 10-week-old mice. The tumor volumes were measured along three orthogonal axes (a, b, and c) and were calculated as follows: tumor volume=a×b×c/2. Mice were pooled and randomly divided into different groups when the tumor reached a volume of approximately 150 mm³ before RT with 5, 10, or 20 Gy, using a Philips RT-250 X-ray generator operating at 250 kVp, 15 mA, with a 1.0 mm copper filter, at a dose rate of 156 cGy/min. The Philips RT250 X-ray generator was regularly calibrated using a Farmer-type ionization chamber to ensure accurate dose delivery. Dose distribution was assessed by performing film dosimetry in a water phantom, which closely mimicked the conditions in the irradiated mice. The films were placed at varying depths to establish the dose gradient and verify the dose uniformity across the target area. The calculated dose distribution showed a consistent delivery to the tumor site with minimal variation. Irradiation doses to healthy tissue were minimized by fully shielding the murine body with lead and exposing only the flank tumor to direct irradiation. Tumors were positioned away from the body to reduce surrounding tissue exposure, aligning with the targeted approach of clinical SBRT. Mice were checked in regular intervals and euthanized before reaching experimental endpoints as defined in the protocols approved by the IACUC of the University of Chicago. For the intravenous tumor injection model, 1×10⁵ tumor cells were inoculated via retroorbital i.v. injection under continuous inhalative anesthesia and mice were sacrificed after 7 and 14 days. For the orthotopic lung tumor model, 0.8×10⁴ LLC^{AR+} and LLC^{AR-} cells were injected percutaneously in 2 μL serum-free DMEM and 2 μL growth factor reduced Matrigel (356231, Corning) under continuous inhalative anesthesia.

Lung tumors were visualized using contrast-enhanced conebeam computed tomography (CBCT) on an X-RAD 225 Cx, following an adapted protocol.⁶⁹ In brief, a CBCT scan was acquired under continuous inhalative anesthesia with 360 projection images (1° per image) with X-ray tube settings of 60 kV and 0.8 mA, and 1.0 mm aluminium filter. In order to enhance soft tissue contrast, 300 μL imeron-300 were injected intravenously 2-3 min prior to CBCT acquisition. Imeron-300 was kindly provided by Mrs. Roberta Fretta, Research Director, Bracco Imaging SpA. CT-guided irradiation was delivered with two beams (0° and 180°) using a 3×3 mm collimator with X-ray tube setings of 220 kV, 13 mA and a 0.15 mm copper filter. The isocenter of the radiation beam was aligned to the center of the contrast-enriching tumor volume.

In vivo treatments: Anti-AREG antibody was administered once every three days at a dose of 1.5 μg/mL blood volume via intravenous (i.v.) injection. Anti-CD47 antibody was administered once every three days via i.v. injection according to the manufacturer's protocol. Recombinant AREG was administered once every three days at a dose of 100 μg/mL blood volume via intravenous (i.v.) injection. Gefitinib was administered daily at a dose of 75 mg/kg body weight by oral gavage.

### BMDM Generation and In Vitro Stimulation

Bone marrow cells from naive mice were isolated by flushing femurs, tibias, and humeri with RPMI-1640 supplemented with 10% fetal bovine serum (FBS) and 1% penicillin-streptomycin. After suspension in ammonium-chloride-potassium (ACK) lysis buffer to lyse red blood cells, the bone marrow cells were filtered through a 40 μm filter. Cells were then resuspended at a density of 1×10⁶ cells per mL and cultured in complete RPMI-1640 medium with GM-CSF (20 ng/mL). Fresh culture media was added on day 3 and cells used for experiments on day 4.

### ELISA

For murine Amphiregulin ELISA assay, blood samples were collected one, three and five days after RT from tumor-bearing mice. Tumor samples were collected on day 5 post RT and homogenized. Samples were spun at 4° C. for 15 mins at 10,000 rpm to remove cells and debris. The concentration of Areg was measured with an Amphiregulin Mouse ELISA Kit (Abcam) in accordance with the manufacturer's instructions.

### In Vitro Live Cell Imaging

Incucyte® live-cell analysis system (EssenBioscience/Sartorius) was used for longitudinal live cell imaging experiments. For in vitro cell proliferation assay, cells were plated at 20% confluence and cell growth was measured every hour for 48 hours. For scratch-wound assay, cells were grown to confluency in 96-well ImageLock® plate (Sartorius) and wounds were generated using 96-Well woundmaker (Sartorius) tool, cells were imaged every 60 mins until they reached confluency. For co-culture assay, tumor cells and BMDM were stained with CellTracker™ Red (CMTPX) and CellTracker™ Green (CMFDA) dye, respectively. Then 1×10⁴ BMDMs were co-plated with 0.5×10⁴ tumor cells for 48 hours and 10× objectives plus phase contrast, green (Ex 441-481/Em 503-544 nm) and red (Ex 567-607/Em 622-704 nm) fluorescent channel were used for imaging.

### In Vitro Analysis of Phagocytic Ratio

Bone marrow cells from wild-type (WT) mice were cultured for 5 days in 20 ng/ml GM-CSF media, refreshed every 2 days. BMDMs were isolated by negative selection using the EasySep Monocyte Isolation Kit (StemCell Technologies, #19861) per manufacturer's instructions. Similarly, WT mouse lung monocytes were isolated post-perfusion, following lung digestion with 1 mg/mL collagenase IV and 200 μg/mL DNase I at 37° C. for 45 minutes. Red blood cells were lysed, and cells were filtered, washed, and isolated with the EasySep Monocyte kit, then cultured in GM-CSF media for 4 days with media changes every 2 days. Bone marrow and lung monocytes were seeded at 30,000 cells/well onto Poly-L-Lysine-coated (Sigma, #P4707) 8-well glass-bottom slides (Ibidi, #80827) and treated with 1 μg/mL polyinosinic-polycytidylic acid (Poly-IC, MedChemExpress, HY-107202) overnight. LLC^{AR+} and LLC^{AR-} cells, pre-treated or not with 250 μg/mL rAREG or 1.5 μg/mL anti-AREG for 24 or 48 h, were grown to 80% confluence, detached, and seeded at 30,000 cells/well overnight on similar slides. The following day, LLC cells were seeded on adherent BMDMs (or vice versa) at 30,000 cells/well, allowing 3 hours of interaction at 37° C. Thirty minutes before the endpoint, cells were stained at 1:100 dilution with anti-mouse Ly6C (PE/Dazzle 594, Biolegend #128044), anti-mouse CD47 (BV421, Biolegend #127527), and anti-mouse SIRPα (Alexa Fluor 647, Biolegend #144028) fixed, and permeabilized with 4% PFA and 0.5% Triton X-100. Following PBS washes, cells were blocked in 5% BSA, stained with primary antibodies for pMyoIIA (Ser19, Cell Signaling, #3671, 1:100) or MyoIIA (Cell Signaling, #3403, 1:100), and secondary anti-rabbit AF488 (2 μg/mL in 1% BSA). Cells were visualized using a SoRa Subdiffraction Marianas Spinning Disk Confocal with 40× or 63×, NA 1. MNPs within 10 μm of tumor cells were evaluated for their “phagocytic ratio”. The phagocytic ratio was calculated by comparing the mean fluorescence intensity (MFI) of phospho-myo-IIA at the phagocytic synapse to that of distant membrane sections for MNPs within 10 μm of a tumor cell. A phagocytic ratio >1 indicated reduced CD47-SIRPα-mediated myo-IIA dephosphorylation, reflecting enhanced actomyosin contractility and phagocytic activity.

### Transwell Migration Assay

Single-cell suspension of LLC^{AR+} and LLC^{AR-} cells (1×10³ cells/insert) in serum-free medium were added into the upper compartment of 24-well transwell plates with 8 μm inserts in polyethylene terephthalate track-etched membranes (Corning). The inserts were placed in plates with DMEM+10% FCS medium. After incubating overnight, wells were gently washed with PBS, migrated cells were fixed with 70% methanol for 10 min, and stained with 0.05% crystal violet. Cells attached to bottom of plates were counted as migrated cells (n=4 FOVs per well, n=3 wells per cell line).

RNA Extraction and Quantitative Real-Time Polymerase Chain Reaction (qRT-PCR)

RNA was extracted using Rneasy Micro Kit (Qiagen) according to the manufacturer's protocols. DNA was synthesized with the High-Capacity cDNA Reverse Transcription Kit (4368814, Applied Biosystems). Real-time polymerase chain reaction (PCR) was performed with SYBR Green PCR Master Mix (4309155, Applied Biosystems), and results were normalized to GAPDH. Relative gene expression was calculated using the 2^{-ΔΔCT} approach. Primer sequences: Areg forward: GGTCTTAGGCTCAGGCCATTA (SEQ ID NO: 1), Areg reverse: CGCTTATGGTGGAAACCTCTC (SEQ ID NO: 2), Cd47 forward: CATGGCCCTCTTCTGATTTC (SEQ ID NO: 3), Cd47 reverse: GGAGGTTGTATAGTCTTCTGATTGG (SEQ ID NO: 4).

### Histology

### H&E Staining

Tissues were fixed with 4% paraformaldehyde for a minimum of 24 hours and sent to the university core facility for embedding and processing. Staining was performed using a Leica Bond RX automated stainer, with the protocol “No Post Primary 1 h Bond DAB Refine”. Slides were scanned using CRi Panoramic SCAN 40× Whole Slide Scanner and image analysis was conducted using QuPath (v0.1.2). To ensure non-overlapping assessment of metastatic lesions, two or three sections were analyzed per lung at experimental endpoint, spaced 300 μm apart. Metastasis size: calculated as the percentage of total lung area occupied by the three to five largest metastases, normalized to the mean size across sections from the same mouse. Mean metastasis numbers also normalized across sections from the same mouse. Due to RT-induced significant reductions in metastasis number the total metastatic area (sum of individual metastases) was not a reliable predictor of increased lesion size. Instead, the mean size of the three to five largest metastatic lesions per lung was used as a representative measure.

### IF Staining

Tissues were fixed in 4% paraformaldehyde for at least 24 hours, then paraffin-embedded and processed at the university core facility. Slides were heated to 55° C. for 45 minutes, deparaffinized with two 2-minute xylene washes, followed by graded ethanol washes (100%, 90%, 75%, 30%), and ddH₂O. For antigen retrieval, slides were incubated at 90° C. in antigen retrieval buffer for 45 minutes, cooled to room temperature, and rinsed with ddH₂O. Tissues were blocked in 5% BSA for 45 minutes in a humidity chamber.

Primary antibodies (pEGFR, Tyr 992, Invitrogen #44786G; Ly6C-PE/Dazzle 594, Biolegend HK1.4, #128044) were applied at 1:100 in 1% BSA, and slides incubated overnight at 4° C. Slides were washed, then incubated with secondary anti-rabbit AF488 (1:500 in 1% BSA) for 1 hour at room temperature. After a final ddH₂O wash, slides were treated with True VIEW Autofluorescence Kit (Vector, #SP8400) per protocol, quenched for 2 minutes, rinsed, stained with 1 μg/mL DAPI for 10 minutes, and washed. Finally, slides were mounted in Vectashield Vibrance Antifade and imaged using a SoRa Subdiffraction Marianas Spinning Disk Confocal with a 40× NA 1.0 objective.

### Tissue Specific AI Cell Segmentation and Logistic Regression Classifier

We developed a custom tissue specific AI-based cell segmentation model using the human-in-the-loop pipeline of Cellpose^{70,71} to analyze immunofluorescence images. Segmentation masks were generated to classify individual cells within the tissue using a custom Python classification pipeline (Code will be made accessible on GitHub). For each segmented cell, morphological features and intensity distributions across different regions of the cell were extracted. A logistic regression classifier was trained on these features, using manually identified monocytes as ground truth, to differentiate monocytes from non-monocytes, using a cutoff value, which was determined by maximizing the Youden's J index in the learning cohort⁷². The model was applied to new images to predict MNP presence. MNP density was subsequently determined by normalizing the detected MNP count to the tissue area in square micrometers, derived from the cytoplasmic channel using Otsu's thresholding⁷³ and the image's physical dimensions obtained from metadata.

### Flow Cytometry

LLC^{AR+} and LLC^{AR-} mice were sacrificed and murine lungs were immediately removed and repeatedly washed in subsequent PBS baths to reduce peripheral blood contamination. Lungs were digested with 1 mg/mL collagenase IV (Sigma-Aldrich) and 200 μg/mL DNaseI (Sigma-Aldrich) at 37° C. for 30-60 minutes. Red blood cells were removed with lysis buffer. Samples were then filtered through a 70 μm cell strainer and washed twice with staining buffer (PBS supplemented with 2% FBS and 0.5 mM EDTA). The cells were re-suspended in staining buffer and were blocked with anti-FcR (2.4G2, BioXcell).

Murine flow cytometry antibodies used were: anti-FcR CD16/CD32 (2.4G2) from BioXcell, CD45 (30-F11), FoxP3 (MF-14) from eBioscience or Invitrogen (US); CD11b (M1/70), CD4 (RM4-5), CD8 (53-6.7), NK1.1 (PK136), CD3 (17A2), F4/80 (BM8), Ly6C (HK1.4), anti-MHCII (M5/114.15.2), PD-1 (29F.1A12), PD-L1 (10F9G2), CD206 (C068C2), CCR7 (4B12), iNOS (CXNFT), CD19 (6D5), Tyr705 STAT3 Phospho (13A3-1) from BioLegend (US); F4/80 (T45-2342), Ly6G (1A8), NK1.1 (PK136), CD8 (53-6.7), F4/80 (T45-2342), and CD11c (HL3) from BD Biosciences (US).

Human flow cytometry antibodies used were: FoxP3 (236A/E7), CD20 (2H7), CD16 (eBioCB16) from Invitrogen, CD4 (SK3), CCR7 (G043H7), PD-1 (EH12.2H7), PD-L1 (B7-H1), HLA-DR (L243), CD34 (561), CD11c (3.9), CD303 (BDCA-2), CD304 (12C2) from Biolegend, CD56 (NCAM16.2), CD141 (1A4) from BD, CD3 (UCHT1), CD33 (WM53), CD45 (HI30), CD8a (SK1) from ThermoFisher.

For pEGFR and AREG staining in murine and human samples, phospho-EGFR (Tyr992) polyclonal antibody (44-786G, ThermoFisher) or Amphiregulin antibody (sc-74501, Santa Cruz) were conjugated using LYNX rapid antibody conjugation kits (Biorad). Dead cells were excluded by Zombie NIR® Fixable Viability Kit (Biolegend, US). For staining one million cells in a 100 μL volume, surface marker antibodies were used at 1:500 dilution (CD16/CD32 blocking was used at 1:10000) and intracellular staining antibodies were used at 1:100 dilution for 30-60 mins at 4° C. Flow cytometry data was acquired using a 5-laser Cytek Aurora and analyzed with FlowJo (v10.8.1, BD Biosciences, US).

### Flow Cytometry Data Analysis

1×10⁴-0.5×10⁵ CD45⁺ cells from each sample were concatenated and used for further downstream analysis. High-dimensional data was visualized using t-distributed stochastic neighbor embedding (t-SNE) in FlowJo 10.8.1 (BD, Franklin Lakes, NJ). Phenograph v2.4⁷⁴ and FlowSOM v3.0.18⁷⁵ were used for unsupervised nearest-neighbor clustering based on phenotypic similarities. FlowJo plugin Cluster explorer v.1.7.4 (BD, Franklin Lakes, NJ) was used for data visualization.

### Bulk RNA Seq of Tumor Cells and Ly6C⁺ MNPs

Tumor cells from single culture or Ly6C⁺ cells from co-culture settings were purified by Aria III cell sorter (BD Biosciences). RNA was extracted using Qiagen Kit (Life Technologies) according to manufacturer's instructions. cDNA library preparation and RNA sequencing were performed by the genomics core facility at The University of Chicago using NovaSeqX (Illumina). The raw sequence reads (110 bp, paired-end) of each sample were in the range of ~22 to 36 million, which have been deposited in NCBI Gene Expression Omnibus.

Raw data quality control was performed using FastQC and filtered with average quality score greater than 30. The reads passing QC were mapped to mouse reference genome (mm 10) with STAR (version 2.7.10b). Gene expression matrix with read count was generated by subread (version 2.0.5). Differentially expressed genes were identified by using the Bioconductor package DESeq2 with FDR <0.1 threshold for hypothesis testing. Dotplots, volcano plots and normalized gene count heatmaps were generated using GraphPad Prism (10.4.0). Gene Set Enrichment Analysis (GSEA) was performed by GSEA_4.3.2.

Analysis of CD45⁺ Cells from Lung Tissue of Tumor-Bearing Mice by Singe Cell RNA-Seq

LLC^{AR+} and LLC^{AR-} flank tumor-bearing mice were treated with 20 Gy once flank tumors reached 150 mm³. Mice were sacrificed and lung tissues were collected five days post-IR, as described above. Single cells were harvested by digestion and CD45⁺ cells were purified by AriaIII cell sorter (BD Biosciences). Four mice were pooled per biological condition (LLC^{AR+}, LLC^{AR+}+20 Gy, LLC^{AR-}, LLC^{AR-}+20 Gy), and GEX (Gene Expression) and HTO (Hashtag Oligos) libraries were generated for each biological condition and demultiplexed using TotalSeq™ hashtag antibodies (Biolegend). For each of the four conditions, both the GEX and HTO libraries were concurrently aligned using cellranger count (Cellranger version 7.1.0 from 10× genomics) with the “include-introns” option set to be “true. All four conditions had high alignment proportions with the reads mapped to the mm10-2020A genome ranging between 91-94%. Barcode processing, filtering, UMI counting and aggregation of sequencing runs were also performed using the Cell Ranger analysis pipeline (version 7.1.0 from 10× genomics). Downstream analyses were primarily performed in Python using the Pegasus package (version 1.8.1)⁷⁶ scanpy package (version 1.8.1)⁷⁷, and the Seurat package (version 4.4.0)⁷⁸.

Gene-expression data for cell barcodes from all four biological conditions were pooled together into a single Pegasus Unimodal object. A multi-step approach was used to perform quality control. For each cellular barcode, two metrics were calculated: total number of genes detected and proportion of mitochondrial UMIs (mito fraction). Cell barcodes with <200 genes or mito fraction >20 were considered to be poor-quality transcriptomes or dying cells and were excluded. Next, we normalized the HTO counts across the filtered cells using the centered log-ratio (CLR) transformation and then demultiplexed using the HTODemux( ) function in Seurat R package and by setting the positive.quintile parameter to 99. All cells with >6000 genes detected or multiple HTO barcodes identified by the HTO classifier were considered multiplets and were filtered out, yielding us a final set of 32,690 CD45⁺ cells.

To assess technical variability between mice arising from the same biological conditions, we first performed dimensional reduction on the entire transcriptome using principal components analysis (PCA). Using the elbow method, the first 50 PCs were deemed to be significant and a t-SNE projection was subsequently generated using these 50 PCs. A mouse-level t-SNE projection was generated by averaging the t-SNE coordinates of all cells originating from each single mouse and this method was used to visualize global transcriptomic shifts induced by the presence of RT as well as AREG.

Next, we took a multi-step approach to clustering cells for cell type identification in the 32,690 CD45⁺ cells using the Pegasus package. First, 18,931 robust genes were identified based on expression in at least 5% of all cells in our dataset. Normalized gene expression counts for robust genes were calculated by scaling counts so that each cell had the same sum of total gene counts (10⁵) and then a log-transformation was performed. Then, genes were ranked as “highly variable” based on having moderate mean expression but high dispersion (computed via the loess smoothing method) using Pegasus. Data dimensionality of the dataset was reduced by selecting the top 4000 most highly-variable, robust genes. Next, PCA was calculated on this dimensionally-reduced transcriptome and top 50 PCs were used for downstream analysis. Batch correction was performed using the run harmony function, which implements the Harmony algorithm⁷⁹, using top 50 PCs as inputs and batch parameter set to the HTO barcode variable which maps a unique barcode to each individual mouse in the experiment. Next, we constructed a k-Nearest-Neighbor (kNN) graph where 30 the nearest neighbors were calculated for each cell. Unsupervised clustering was performed using the Leiden algorithm, a modularity optimization algorithm, using a resolution parameter of 0.8 and yielded 21 cell clusters of 11 major lineages. UMAP (Uniform Manifold Approximation and Projection) was performed and used to visualize the clusters in a 2-D space. To identify significant cluster specific gene markers, we performed differential gene expression using the Mann-Whitney U (MWU) test to compare gene expression in the cluster of interest against all other clusters and retained only the genes with MWU q-value <0.05. Cell type labels were assigned by a combination of manual curation by immunology experts and auto-annotation tools in Pegasus.

For mononuclear phagocyte (MNP) focused-analyses, MNP clusters (4,095 cells) were selected and previously described steps were repeated (PCA, kNN, leiden with resolution of 0.8) to yield 17 cell clusters. Marker genes were identified as previously described using the MWU test to compare clusters and using a q-value of 0.05 to threshold out significant genes. Clusters were visualized using UMAP. Biological condition-specific MNP densities were visualized by calculating the kernel density estimate of the UMAP embedding using the kdeplot function from the Python seaborn library with thresh set to 0 and levels set to 15.

Monocyte populations of interest were manually selected from the MNP population (2,600 cells) and underlying differentiation/developmental relationships between these populations were explored by computing the diffusion map (top 100 diffusion components) using the Pegasus package (default parameters were used). Pseudotime was computed in Pegasus to estimate relative progression of cells along the differentiation/developmental processes identified in the diffusion map. Next, the top 10 diffusion components of monocyte populations were input into the Slingshot R package⁸⁰ to infer likely trajectories. The slingshot function and getLineages with default parameters and monocyte population 1 as starting population were utilized.

For RNA velocity analysis, the fraction of spliced and un-spliced mRNAs for all detected genes was calculated using the Velocyto package (version 0.17.17)⁸¹. Using these fractions, RNA velocities for the manually-selected 2,600 MNP cells, were then computed using the sc Velo package (0.3.2)⁸². Specifically, the top 4,000 genes were selected and moments were calculated with the number of principal components set to 30 and the number of neighbors set to 15. The dynamical method was used to learn the full transcriptional dynamics of splicing kinetics.

While comparing mean gene expression between monocyte clusters (either individual gene markers or gene signatures), we applied a more stringent cut-off and filtered out cells with mito fraction >5 to enable the most accurate comparisons. Gene signature calculations were performed using manually curated signatures from literature as well as gene sets from the Reactome⁸³ and the KEGG gene sets⁸⁴. Single-cell signature scoring was performed using a well-established gene-scoring methodology⁸⁵. The gene-scoring method employs a strategy aimed at minimizing the impact of technical variability while amplifying biological signals. It categorizes all genes in the genome into 50 bins and then assesses how many genes from the gene signature E fall into each bin. Next, it computes a weighted sum of normalized counts for each gene in signature E. This sum is then subtracted from a weighted sum derived from 1000 randomly generated “null” signatures, which are gene signatures of the same size as the original set E and share its bin distribution

### TCGA Data Analysis

TCGA data was acquired and analyzed in part using the Xena Platform⁸⁶. For analysis of the LUSC cohort, gene expression was divided into high and low expression based on the median gene expression of all samples. Samples in the lowest and highest groups received a score of −1 and 1, respectively. Scores were added for each sample. Samples were filtered for patients receiving radiation therapy. Survival data from these groups were then compared and significance calculated using the ggsurvplot function in R.

### Statistical Analysis

Sample sizes were modelled after those from existing publications regarding in vitro immune killing assays and in vivo tumor growth assays, and an independent statistical method was not used to determine sample size. For tumor growth data, descriptive statistics of tumor size were summarized by treatment group at each time point. Tumor growth curves were plotted over time by treatment. Two-way ANOVA tests were used to analyze the tumor growth curve. For both one-way and multiple comparisons were accounted for by Šídák's and Tukey's multiple comparisons test. For subcutaneous tumors, mice were taken off study when individual tumor volumes were ≥2,000 mm³. For lung metastasis models, mice were taken off study when a relevant worsening of body condition score (BCS) or 20% loss of body weight occurred. The survival curves were analyzed by Kaplan-Meier survival analysis with the log-rank (Mantel-Cox) test. Flow cytometry data were summarized, presented using descriptive statistics for each treatment, and compared across treatment groups using One-way ANOVA followed by Šídák's multiple comparisons test or unpaired t-tests. Statistical figures were prepared using Prism v8.4.0, GraphPad Software). P values as indicated in figures.

### Results

### Radiotherapy Induces Amphiregulin Expression and Growth of Distant Metastases

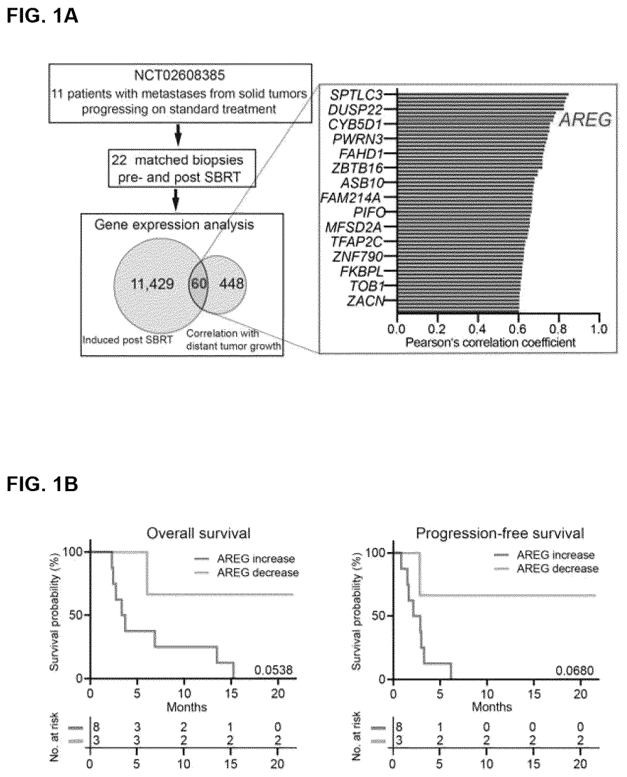

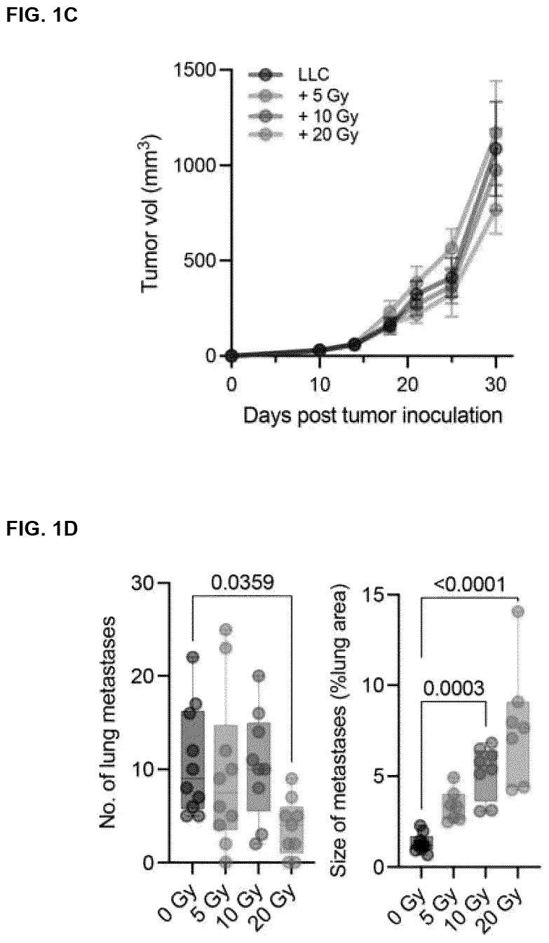

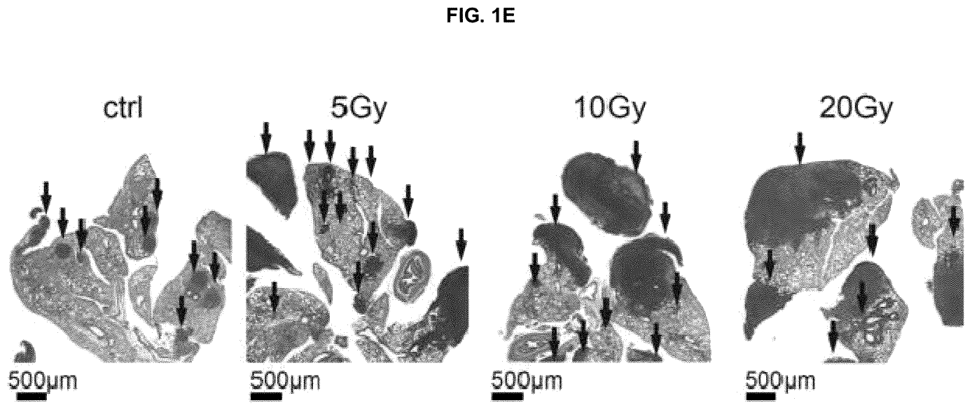

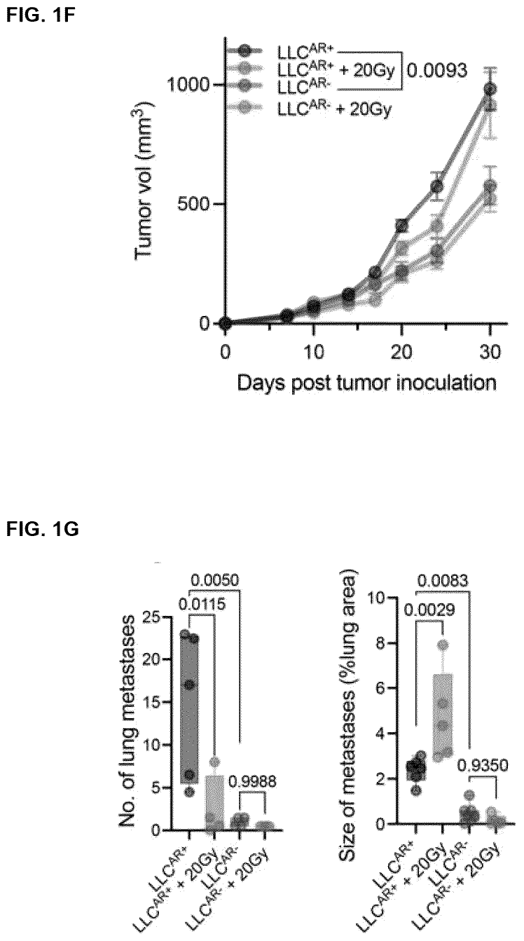

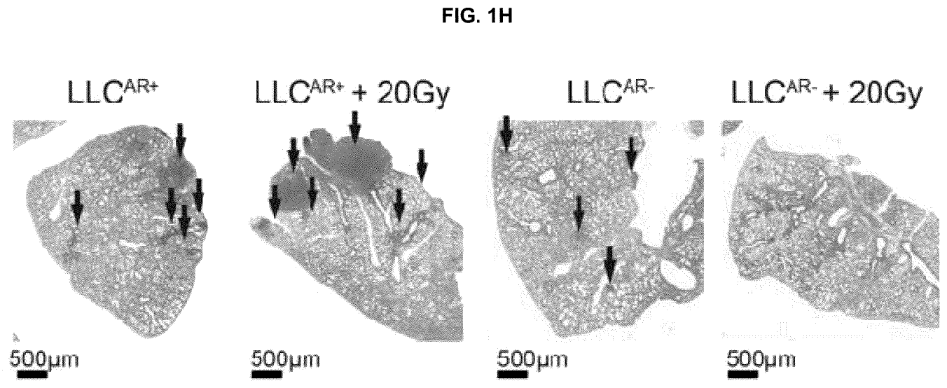

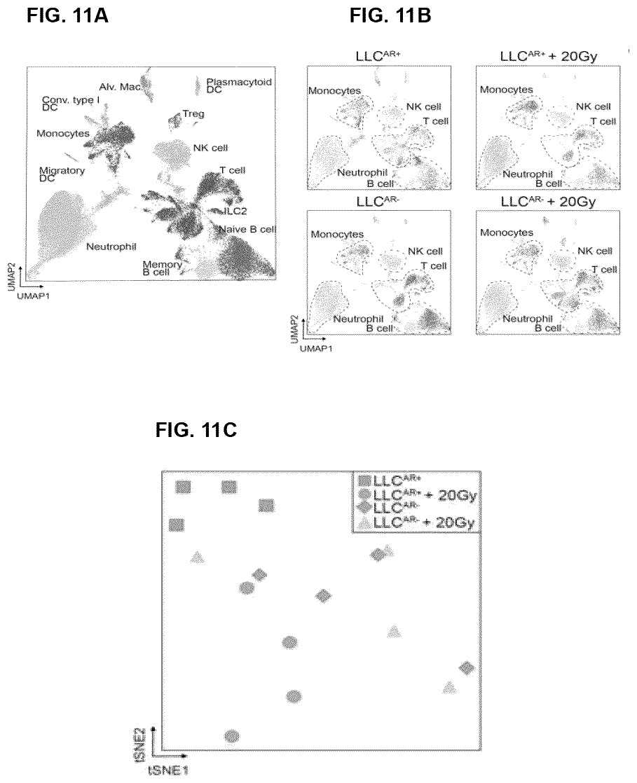

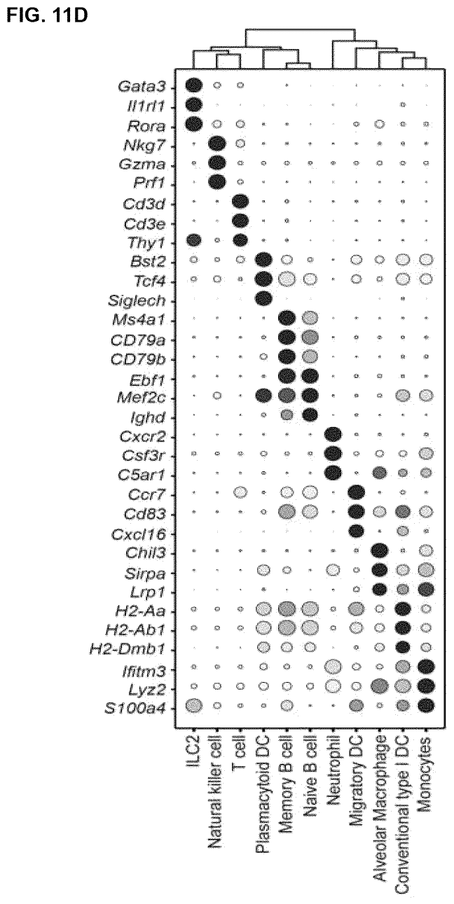

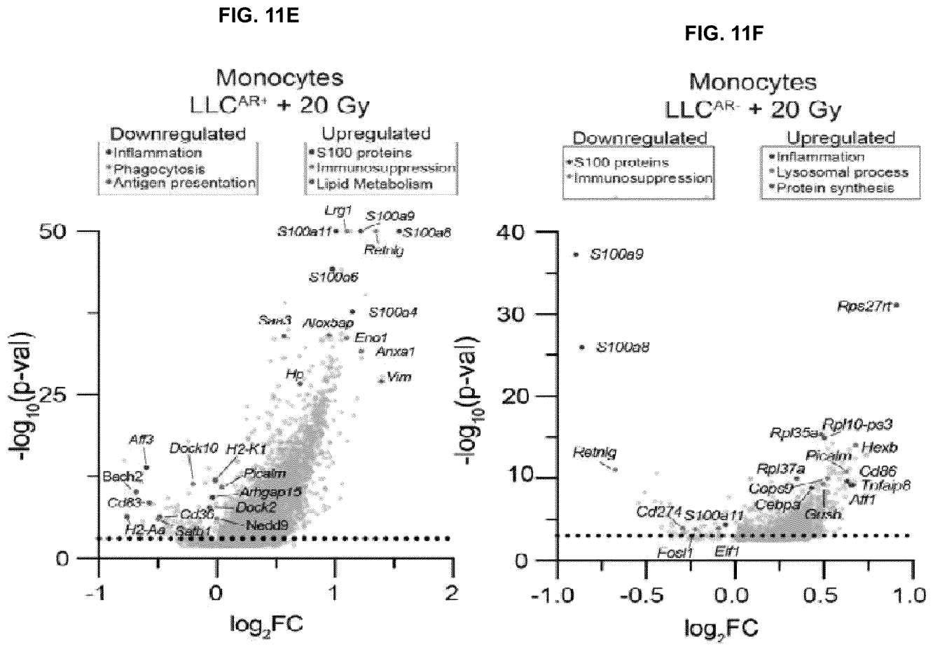

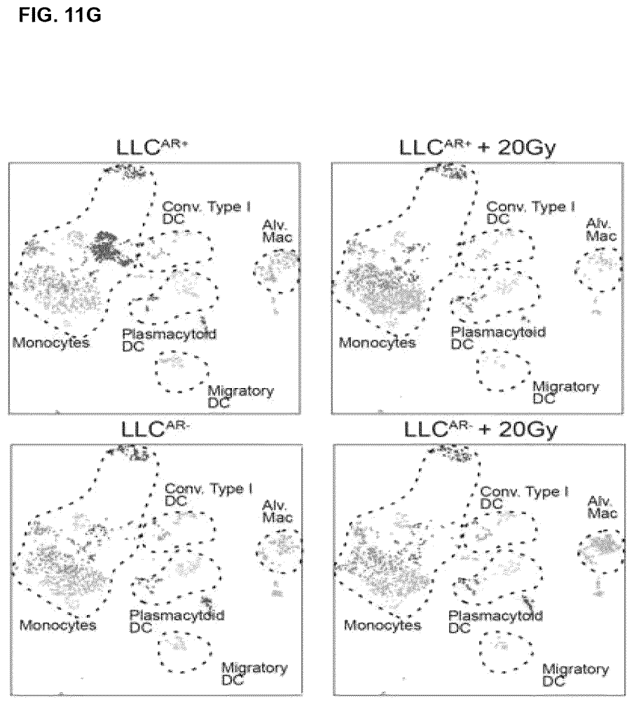

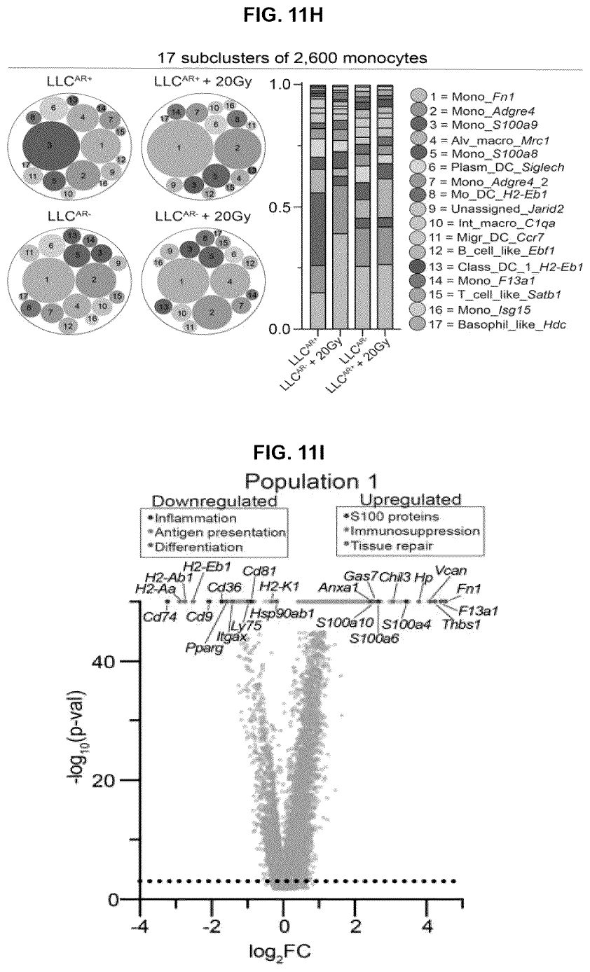

We profiled the gene expression of patients with advanced solid tumors who received SBRT to multiple metastatic sites (clinical trial NCT02608385)^{21,22}. In 22 matched pre- and post-RT biopsies from irradiated metastases, we identified genes that (i) increased in expression following SBRT, and (ii) positively correlated with progression of distant unirradiated metastases. We found 60 overlapping genes that were induced by SBRT and positively correlated with distant tumor growth (FIG. 1A, Table 1). Among these, the EGFR ligand AREG was involved in four of the top 20 upregulated gene pathways that correlated with distant tumor progression (FIG. 11A). Stratifying the patient cohort by the change in AREG expression in response to RT demonstrated a shorter progression-free survival (PFS) and overall survival (OS) in patients whose tumors exhibited increased AREG expression following SBRT (FIG. 1B).

TABLE 1
SBRT induced genes positively correlated
with distant tumor growth

| | Pearson's correlation with |
|---|---|
| Gene Name | distant tumor progression |
| ABHD4 | 0.71890049 |
| ADH5 | 0.695443343 |
| ANKEF1 | 0.618322839 |
| AP4S1 | 0.621263293 |
| AREG | 0.774671685 |
| ASB10 | 0.67918294 |
| ASB7 | 0.669247568 |
| C1orf146 | 0.656797436 |
| CCKAR | 0.645008404 |
| CKMT2-AS1 | 0.610322623 |
| CLUAP1 | 0.728515502 |
| CNTN2 | 0.604697585 |
| COQ6 | 0.737378255 |
| CRELD1 | 0.785550804 |
| CYB5D1 | 0.75682963 |
| DHODH | 0.651118506 |
| DNAJC28 | 0.674743997 |
| DUSP22 | 0.824811142 |
| FAHD1 | 0.72341445 |
| FAM214A | 0.66925609 |
| FAM215A | 0.720424974 |
| FBXW9 | 0.731125312 |
| FKBPL | 0.618465385 |
| GPKOW | 0.659638635 |
| GSTZ1 | 0.666212782 |
| HEATR9 | 0.606255655 |
| HYAL4 | 0.633753772 |
| IFT80 | 0.753180676 |
| KCNH7 | 0.657330913 |
| MDH1B | 0.604352885 |
| MFSD2A | 0.655923503 |
| NMRK1 | 0.625604458 |
| NUDT6 | 0.647096576 |
| ORAOV1 | 0.666657545 |
| PIFO | 0.664655901 |
| PRG2 | 0.615861907 |
| PRPF39 | 0.670011446 |
| PWRN3 | 0.744186628 |
| RBMXL3 | 0.679928483 |
| ROGDI | 0.673740997 |
| RPS6KA5 | 0.605659808 |
| SCARNA20 | 0.825924699 |
| SCN11A | 0.602361846 |
| SLC44A1 | 0.837693943 |
| SNORD8 | 0.62915309 |
| SPTLC3 | 0.848624416 |
| STK32A | 0.719235989 |
| SVBP | 0.753691962 |
| TFAP2C | 0.634506231 |
| TIGAR | 0.745267928 |
| TMEM267 | 0.696708244 |
| TOB1 | 0.610669795 |
| TRIM6-TRIM34 | 0.619235769 |
| WRAP73 | 0.831334544 |
| ZACN | 0.604828877 |
| ZBTB16 | 0.71879623 |
| ZNF786 | 0.611211437 |
| ZNF790 | 0.62589809 |
| ZNF805 | 0.628419144 |
| ZRANB3 | 0.776249616 |

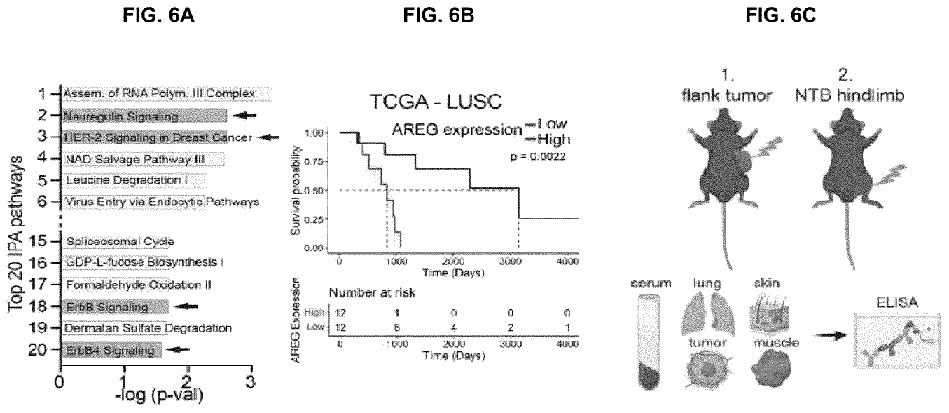

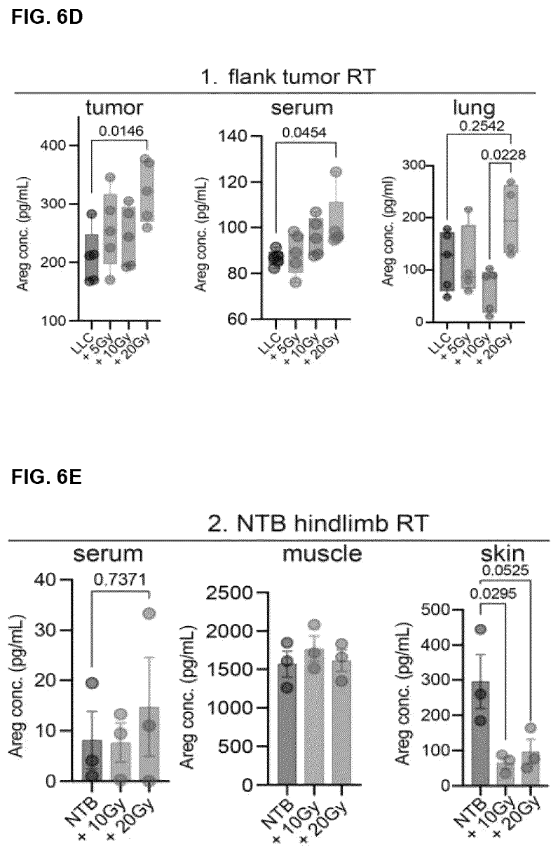

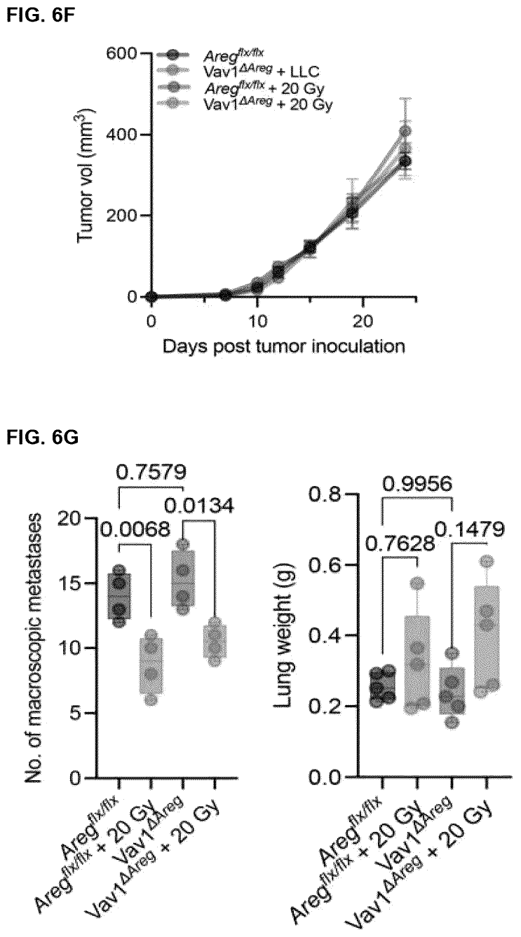

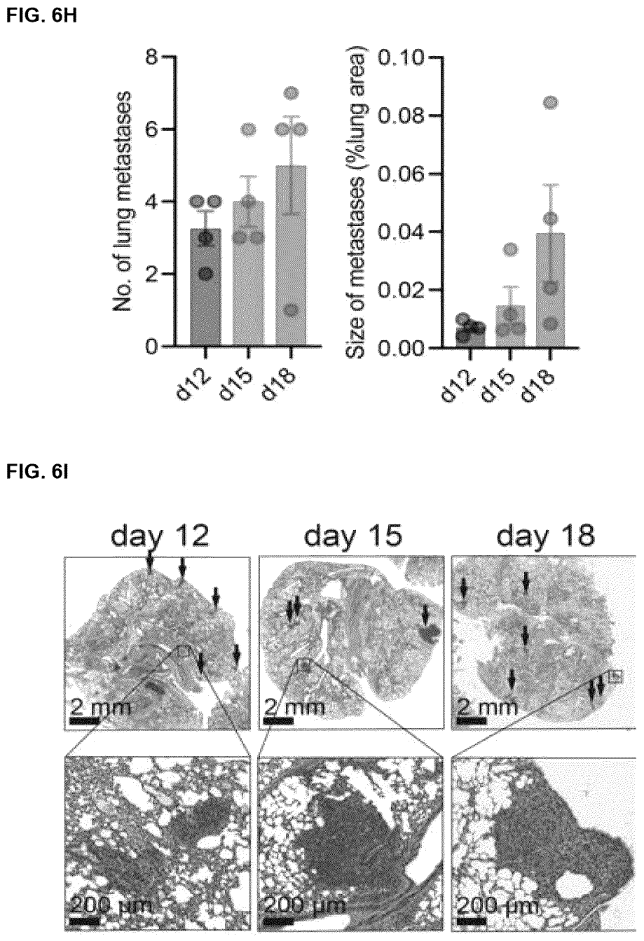

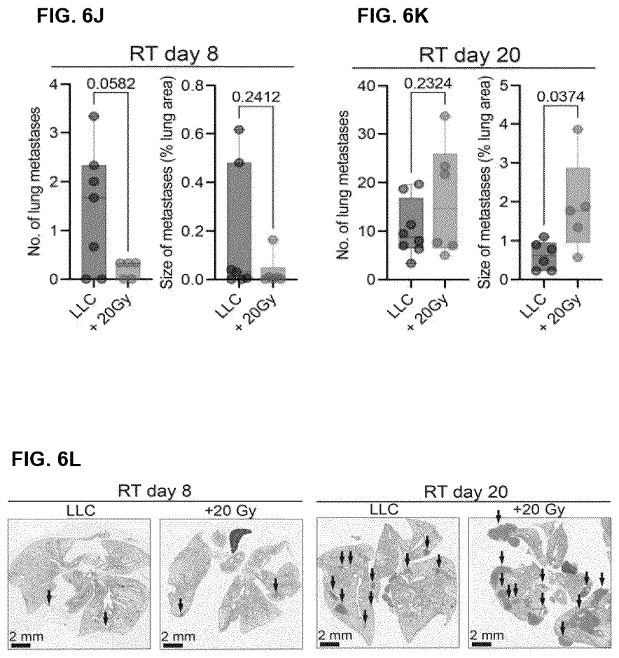

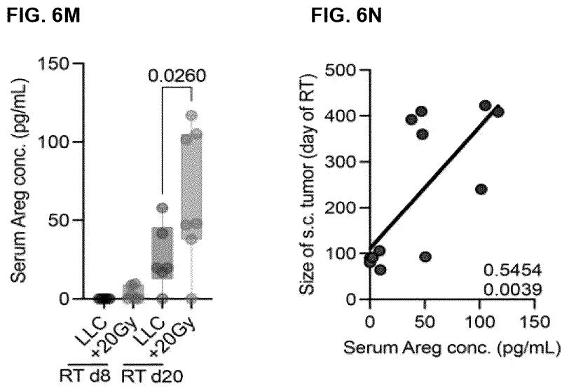

We utilized the murine Lewis lung carcinoma (LLC) model of spontaneous lung cancer metastasis to characterize the mechanisms by which RT-induced AREG expression contributes to distant metastasis. LLC tumor cells were subcutaneously (s.c.) implanted in the flank of C57BL/6 wildtype (WT) mice and treated with local RT (5, 10, and 20 Gy). The resulting tumors showed a high degree of radiation-resistance at these doses (FIG. 1C). Although 20 Gy decreased the number of lung metastases, both 10 Gy and 20 Gy significantly increased the mean size of lung metastases compared with metastases derived from non-irradiated tumors (FIGS. 1D-1E). Irradiation of LLC flank tumors induced a significant, dose-dependent increase of local and systemic AREG concentration with highest levels at 20 Gy, which was absent following RT in non-tumor bearing (NTB) mice, indicating that AREG upregulation is specific to irradiated tumor tissues (FIGS. 6C-6E). Similar flank tumor and lung metastasis size was observed in WT mice and in Vav1^{-Areg} mice, in which all hematopoietic and endothelial lineages lack Areg²⁴, which reinforced that although a range of tissues can produce AREG, tumor cell-derived AREG plays a central role in mediating the metastasis phenotype (FIGS. 6F-6G). In addition, we verified that lung metastases were present at the time of systemic RT-induced AREG upregulation (five days post IR) (FIGS. 6H-61). While early irradiation of smaller tumors on day 8 reduced the number and size of lung metastases (FIG. 6J), a significant increase in metastasis size of larger tumors (IR day 20) coincided with greater systemic AREG levels post-IR (mean±SEM flank tumor sizes 79.95±6.10 and 415.80±30.02 mm3, respectively, FIGS. 6K-6M). A positive correlation was observed between tumor size at the time of RT and the induction of serum AREG five days later (Pearson R=0.5454, p=0.0039), suggesting that larger irradiated tumors secrete more AREG (FIG. 6N).

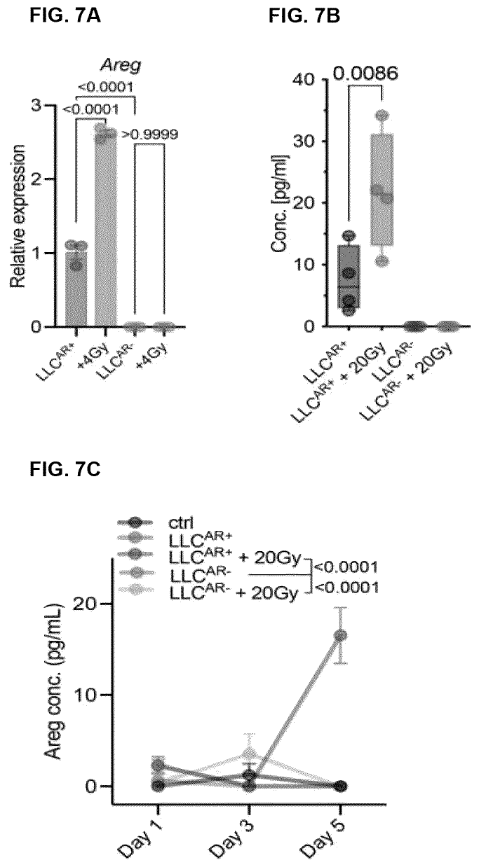

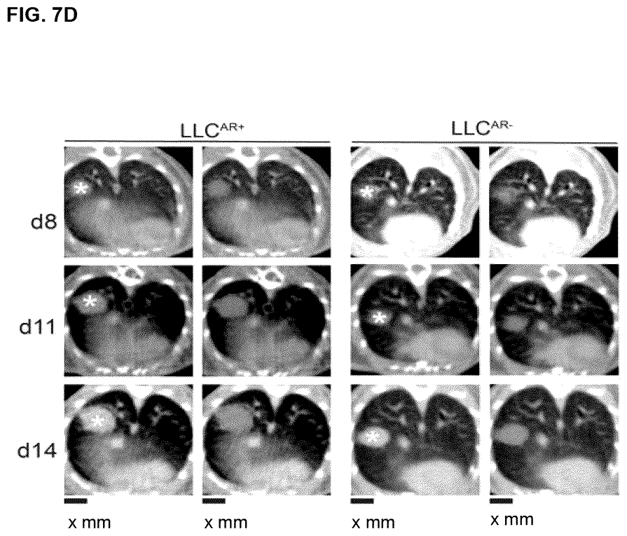

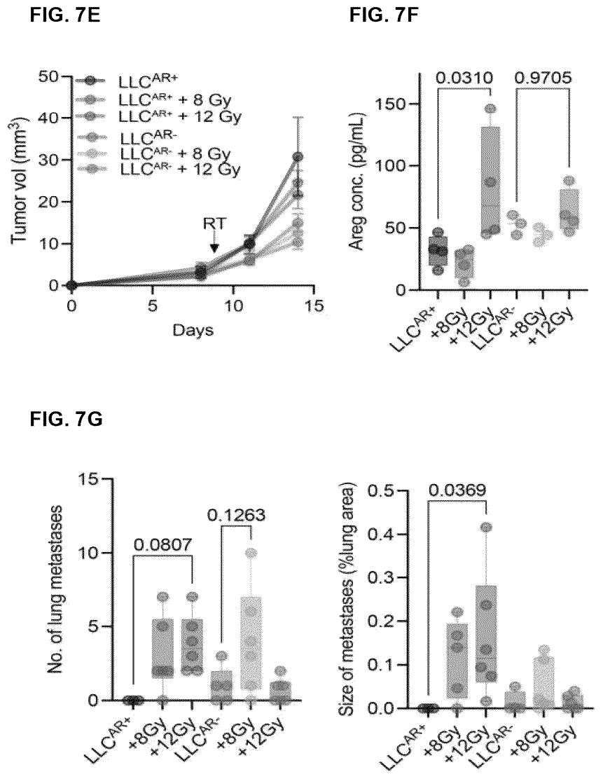

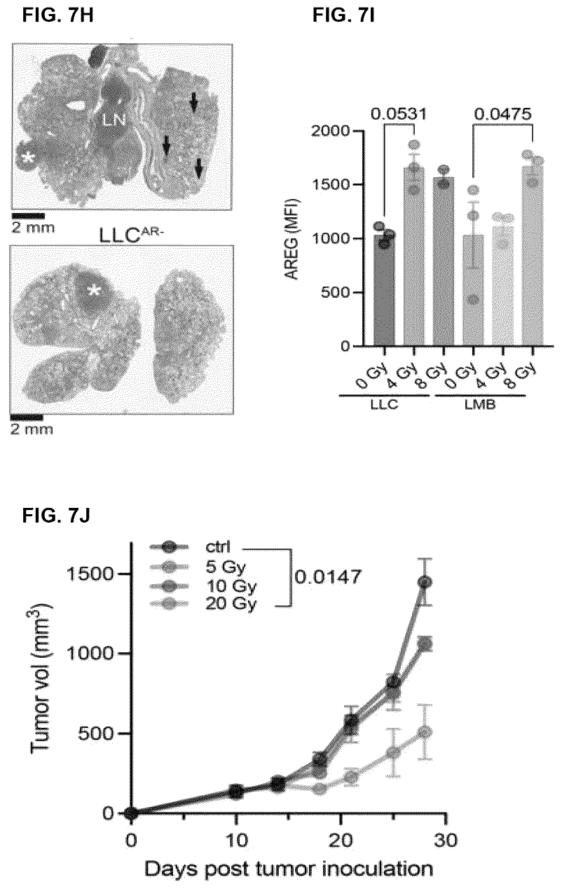

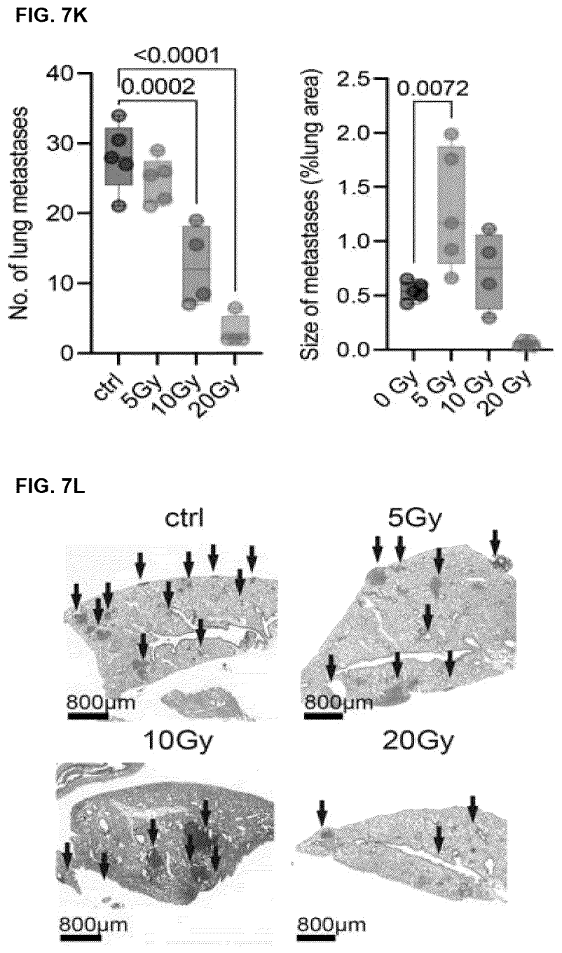

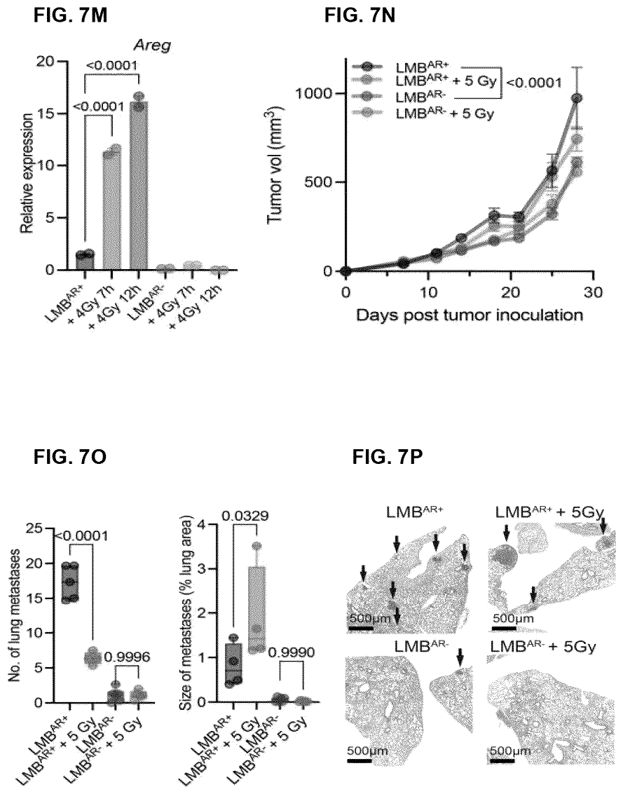

A CRISPR-Cas9 genome-edited knockout (KO) of Areg in LLC cells expressing red fluorescent protein (RFP, hereafter referred to as LLC^{AR+} and LLC^{AR-}) was generated to evaluate the impact of AREG on metastasis growth and response to irradiation. LLC^{AR-} did not upregulate AREG after RT (FIGS. 7A-7C). Non-irradiated LLC^{AR-} tumors exhibited slower tumor growth than LLC^{AR+}, while a high degree of radiation-resistance persisted in both flank tumors (FIG. 1F). At the same time, LLC^{AR-} tumors resulted in significantly smaller and fewer lung metastases and the size of LLC^{AR-} lung metastases did not increase following flank tumor RT (FIGS. 1G-1H). These results were reproduced in an orthotopic model, in which CT-guided irradiation of intraparenchymal lung tumors resulted in increased systemic AREG concentration and size of contralateral lung metastases in LLC^{AR+}, but not in LLC^{AR-} (FIGS. 7D-7H).

In the breast cancer cell line E0771-LMB²⁵ (hereafter LMB), which spontaneously metastasizes to the lung and also upregulates Areg in response to RT (FIG. 7I), 20 Gy suppressed both flank tumor and lung metastasis growth. However, a single dose of 5 Gy, unable to suppress LMB flank tumor growth, led to a significant size increase of lung metastases compared with untreated controls, suggesting that distant metastasis proliferation may be particularly pronounced in the setting of ineffective local tumor control (FIGS. 7J-7L). Like the effects observed in LLC, CRISPR-mediated Areg KO prevented radiation-induced Areg upregulation and significantly delayed the growth of flank tumors without affecting the tumor's response to RT (FIG. 7N). At the same time, LMBAR-demonstrated a reduced number and size of spontaneous lung metastases and RT-related metastasis growth was absent (FIGS. 7O-7P). Thus, while local RT can decrease the number of lung metastases, it can also increase the size of established lung metastases through AREG upregulation.

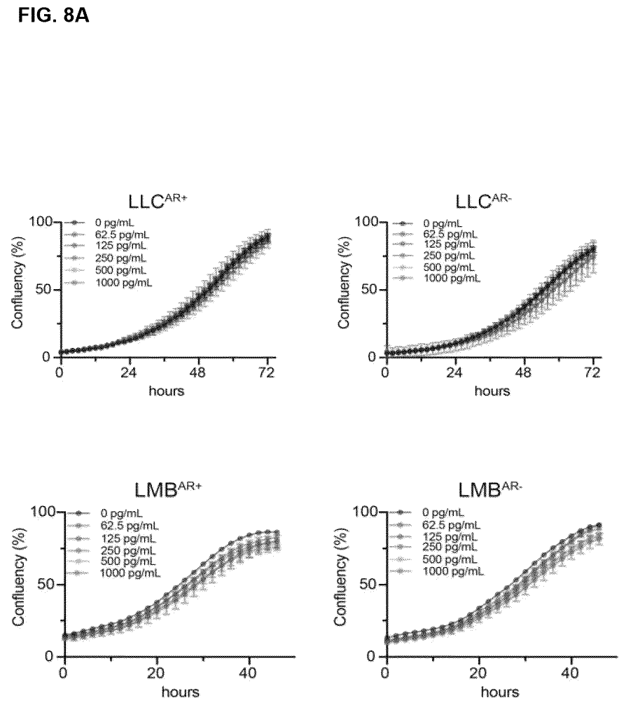

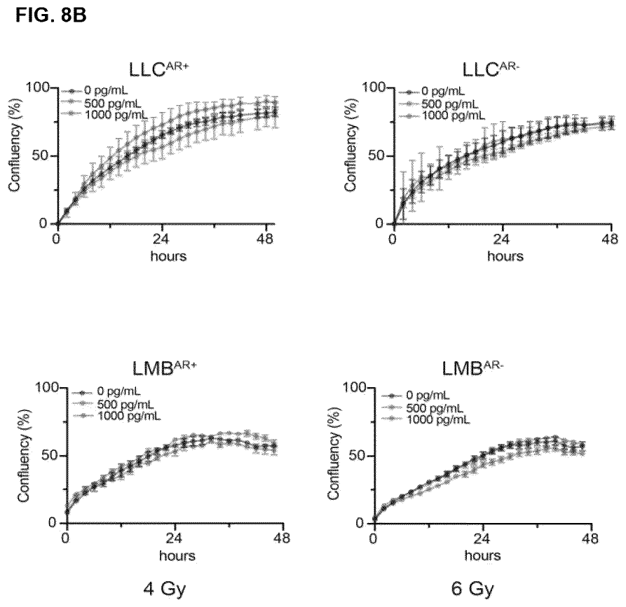

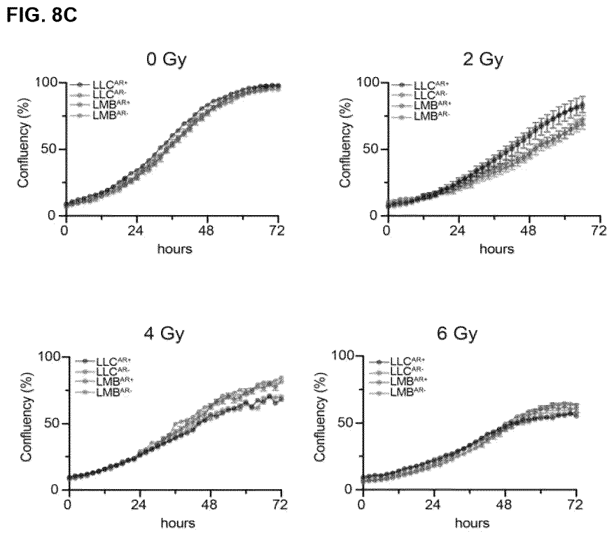

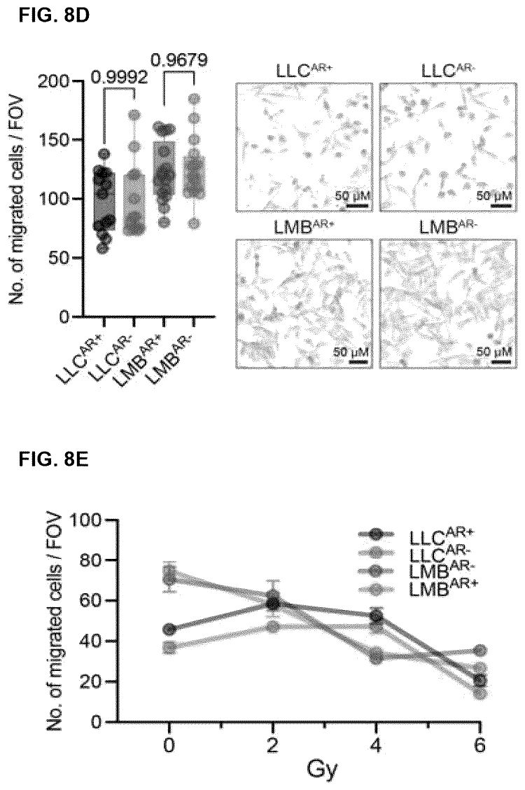

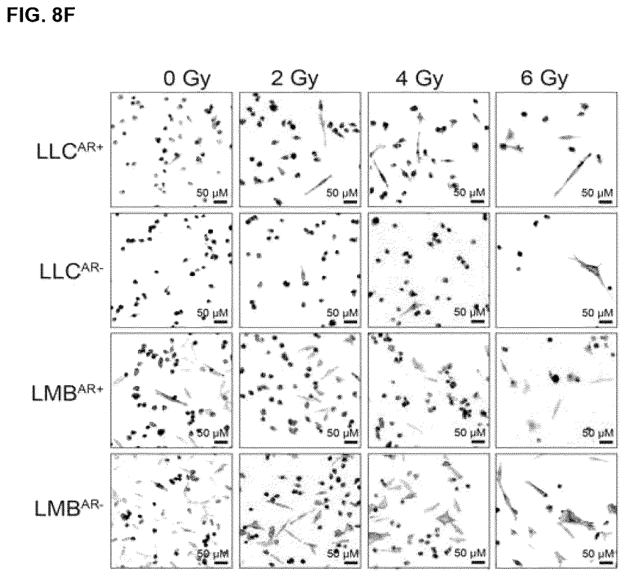

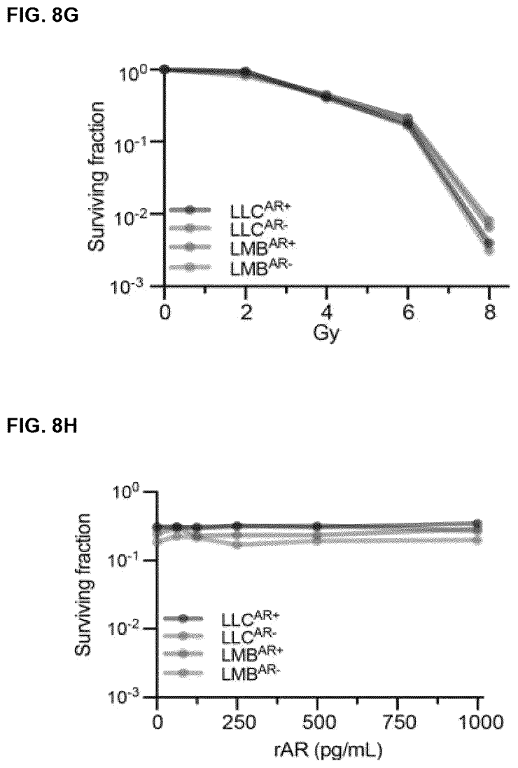

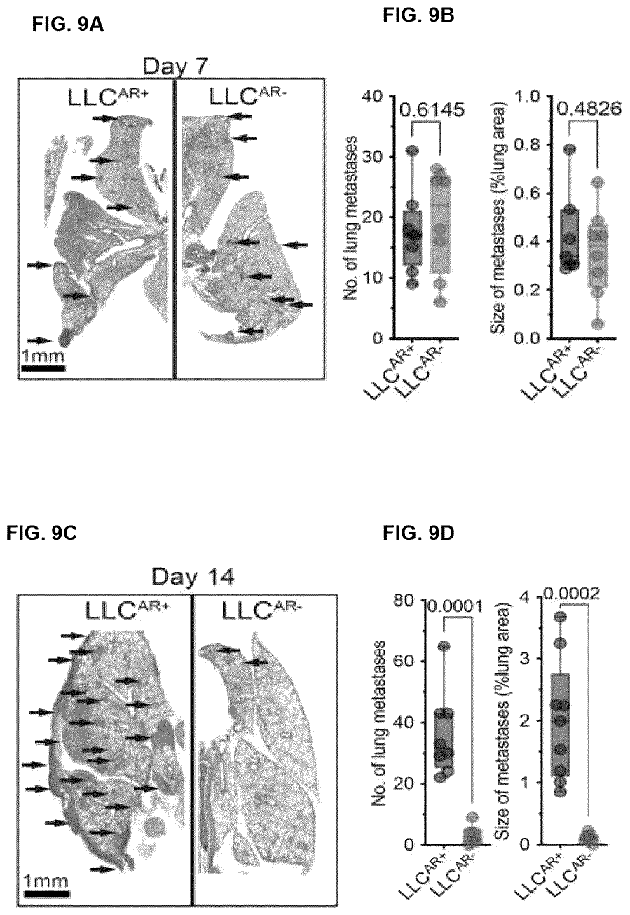

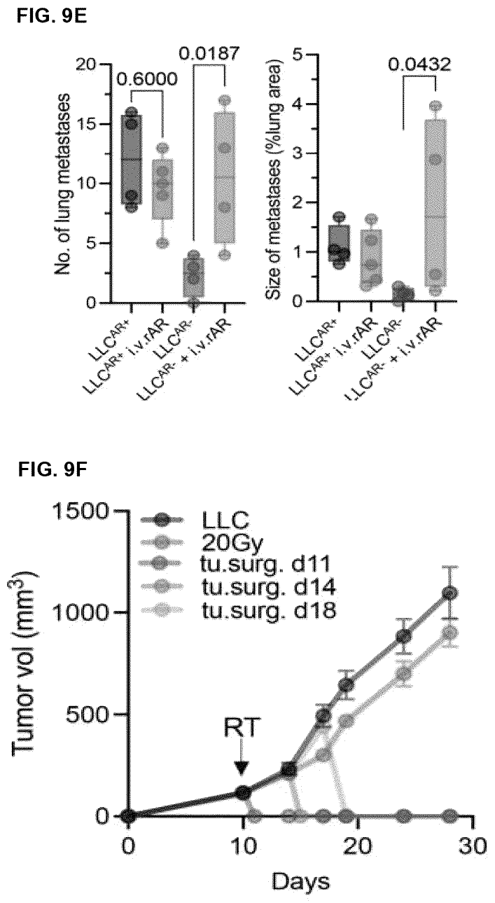

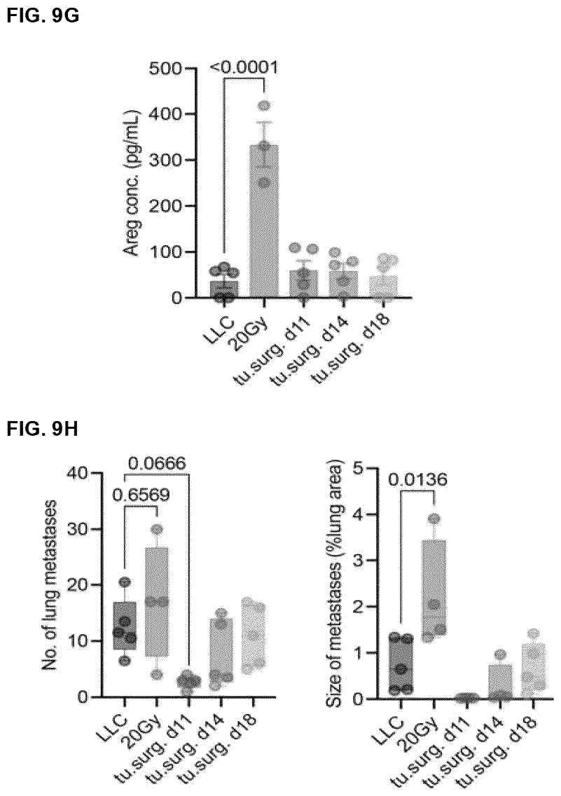

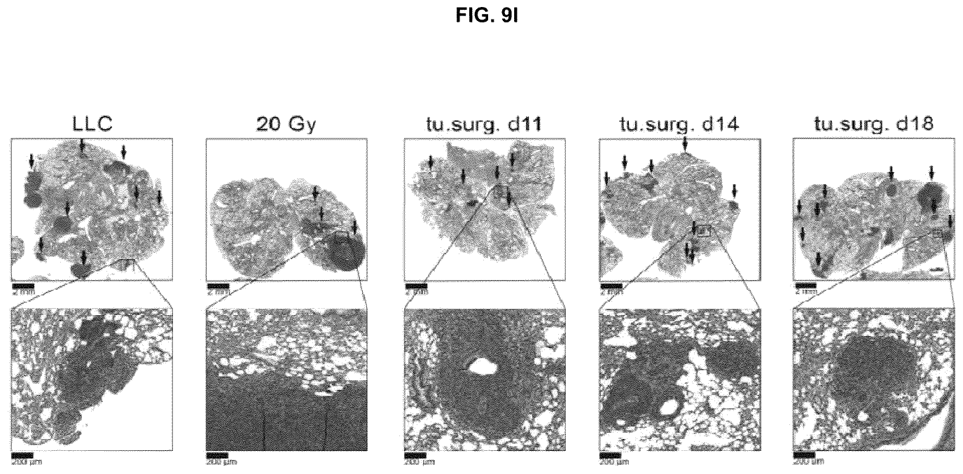

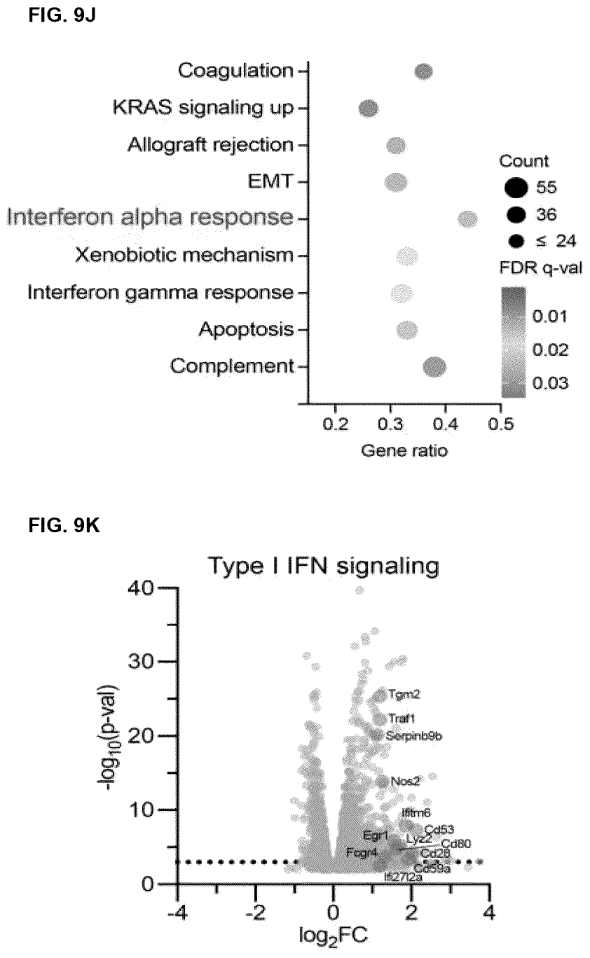

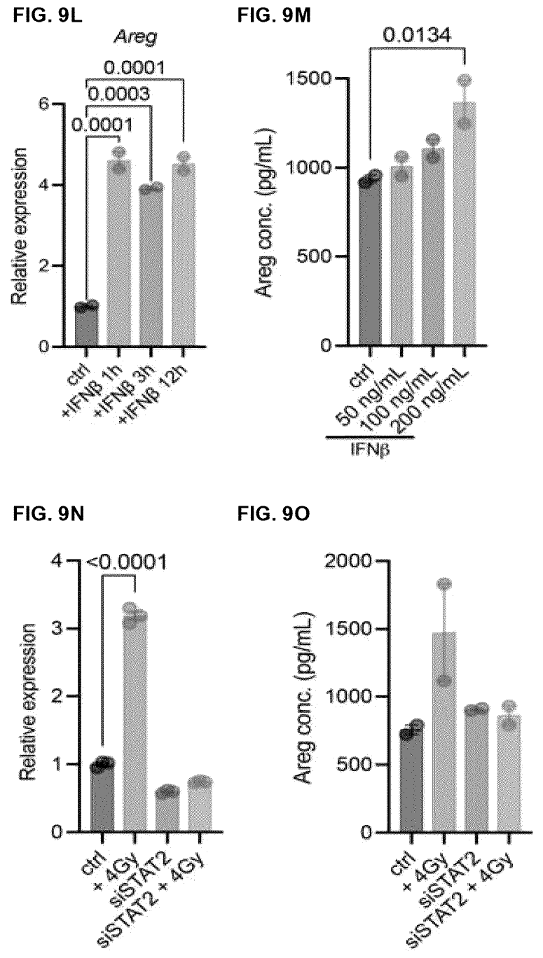

Areg KO and recombinant AREG treatment did not impact the proliferative (FIG. 8A) or migratory (FIG. 8B) capacity of AR+ and AR− LLC and LMB tumor cells in vitro. Similarly, RT effects on proliferation, migration and clonogenic survival were unchanged (FIGS. 8C-8H). To delineate whether the seeding or growth of lung metastases is impacted by tumor cell-derived AREG, we injected LLC^{AR+} and LLC^{AR-} cells intravenously (i.v.). Both LLC^{AR+} and LLC^{AR-} cells extravasated and formed micrometastases 7 days post-injection (FIGS. 9A-9B). Fourteen days after i.v. injection, however, both the number and size of LLC^{AR-} metastases were significantly reduced compared to LLC^{AR+} (FIGS. 9C-9D). To recapitulate the AREG-induced phenotype, we employed intravenous (i.v.) injection of recombinant AREG (rAR), which significantly increased the size of LLC^{AR-} lung metastases, implying that in the absence of tumor-derived AREG, its supplementation can drive lung metastasis growth (FIG. 9E). We next explored whether this mechanism is unique in the context of radiation or whether other types of tissue injury similarly elicit lung metastasis growth. Surgical resection of the flank tumor did not increase systemic AREG levels nor lung metastasis size (FIGS. 9F-9I). Mechanistically, RT-induction of AREG was mediated by type I IFN signaling, as reported in previous studies. 26 RNA-seq of LLC^{AR+} cells following RT identified type I IFN signaling among the top upregulated pathways (FIGS. 9J-9K). Furthermore, Interferon β (IFNβ) treatment significantly increased AREG RNA and protein levels in LLC cells, an effect attenuated by knockdown of the type I IFN-related transcription factor STAT2 (FIGS. 9L-9O).

### Example 2: Radiation-Induced AREG Modulates the Myeloid Immune Landscape in NSCLC Patient PBMCs and a Murine Lung Metastasis Model

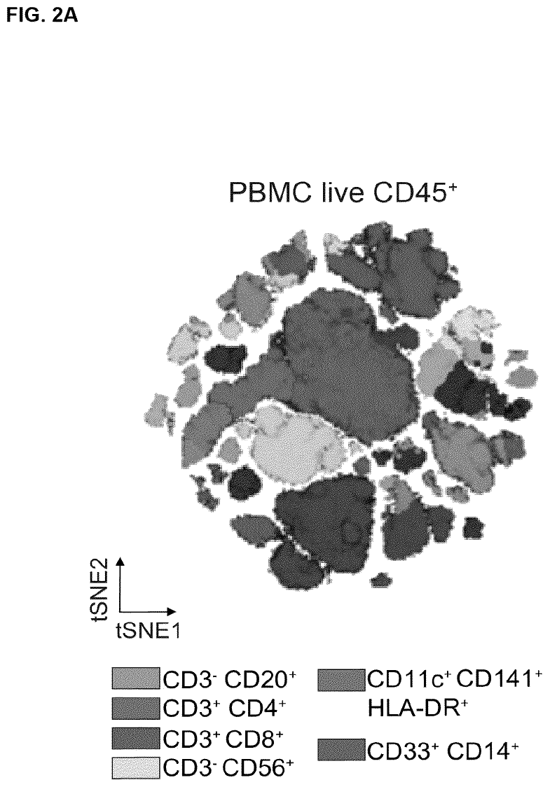

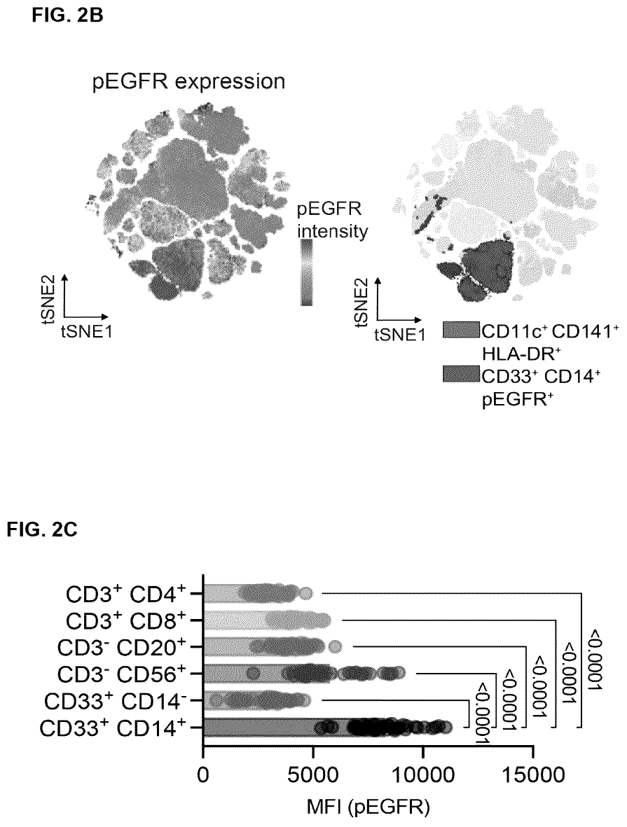

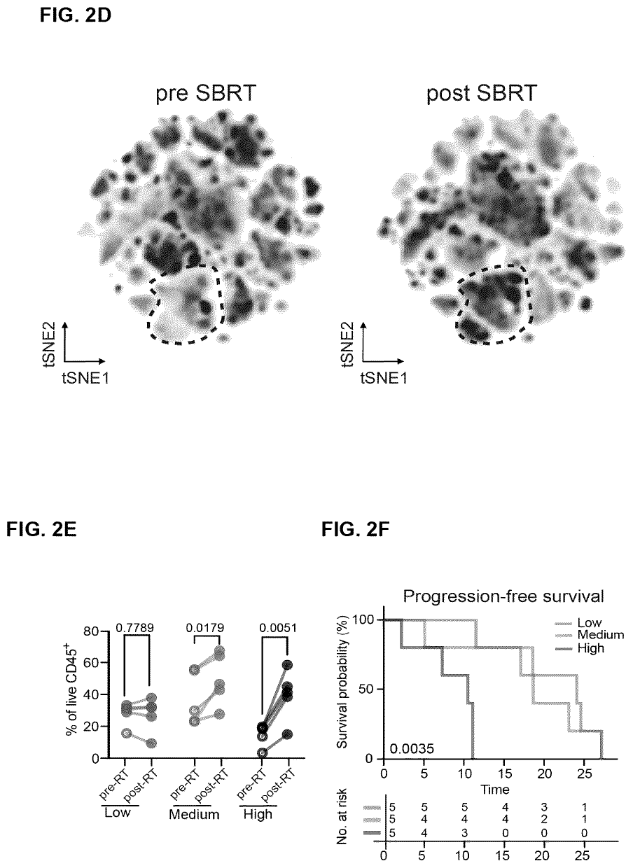

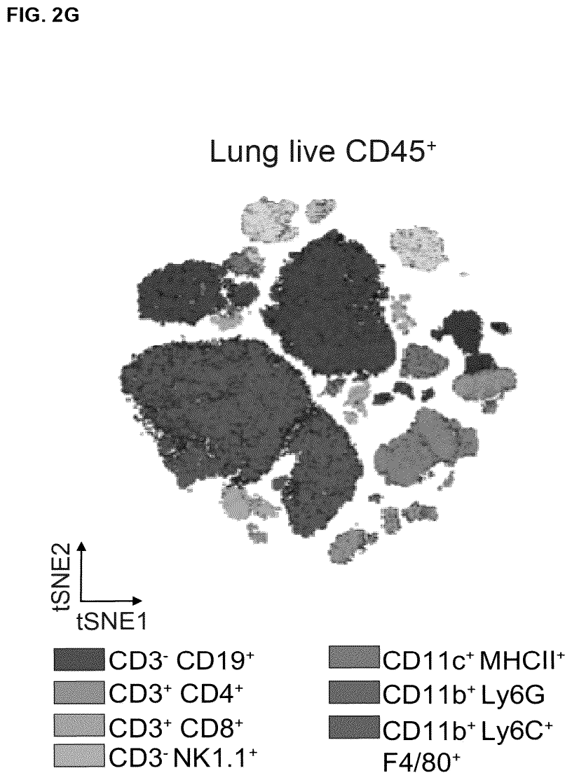

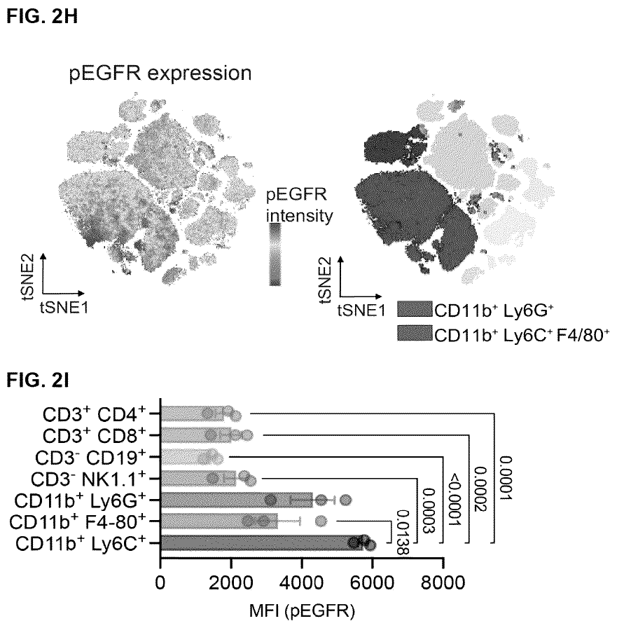

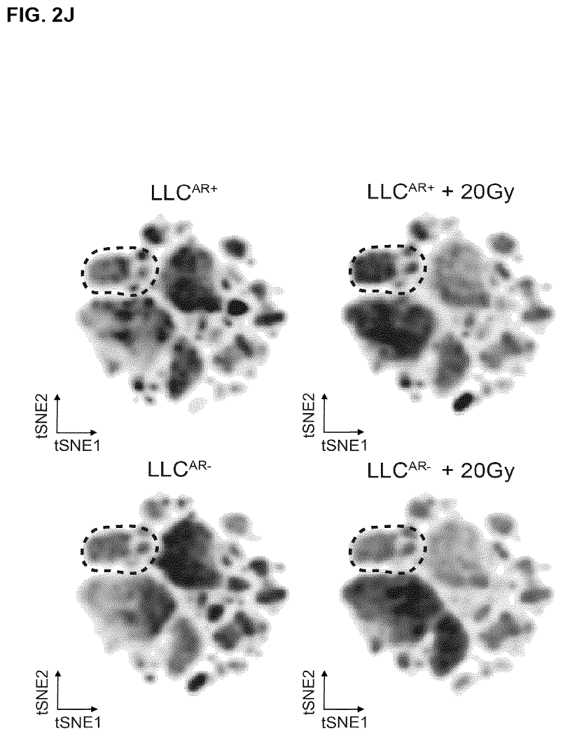

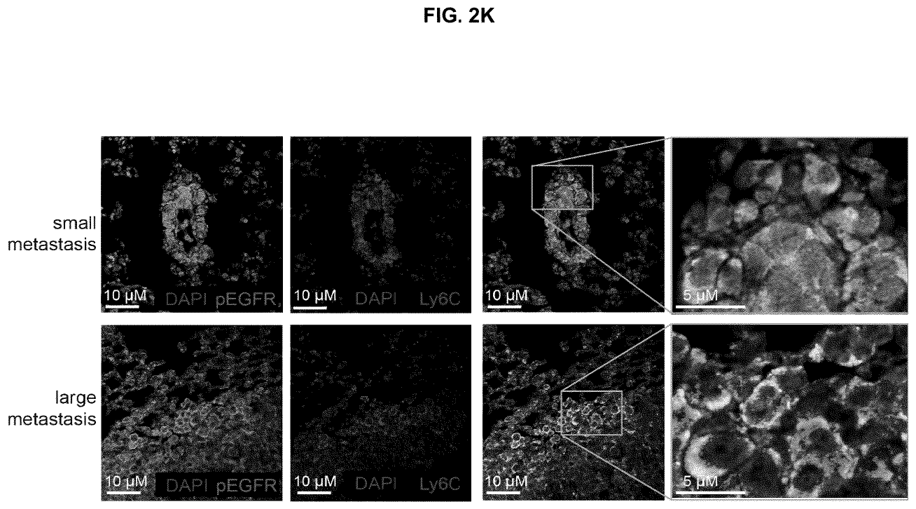

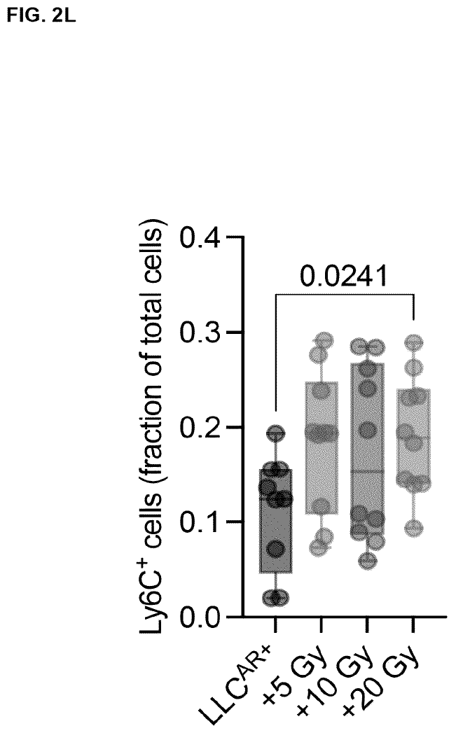

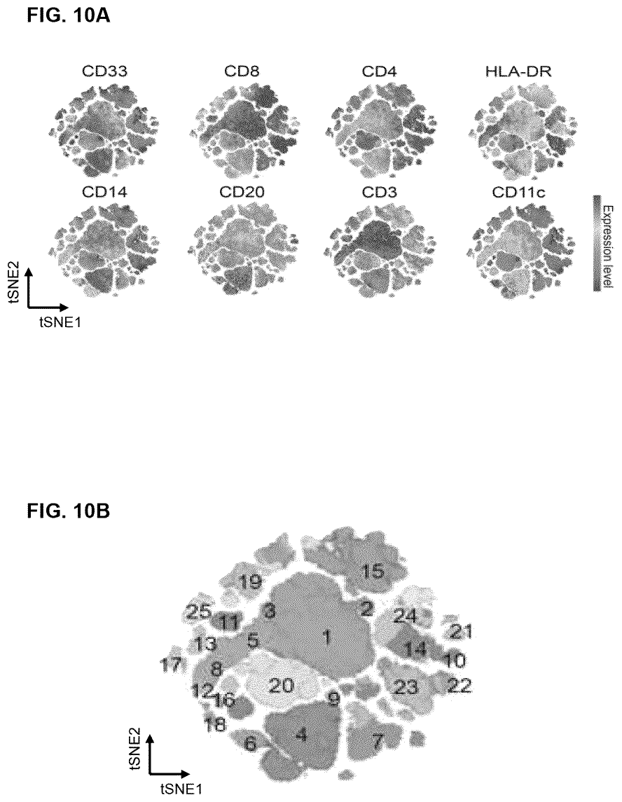

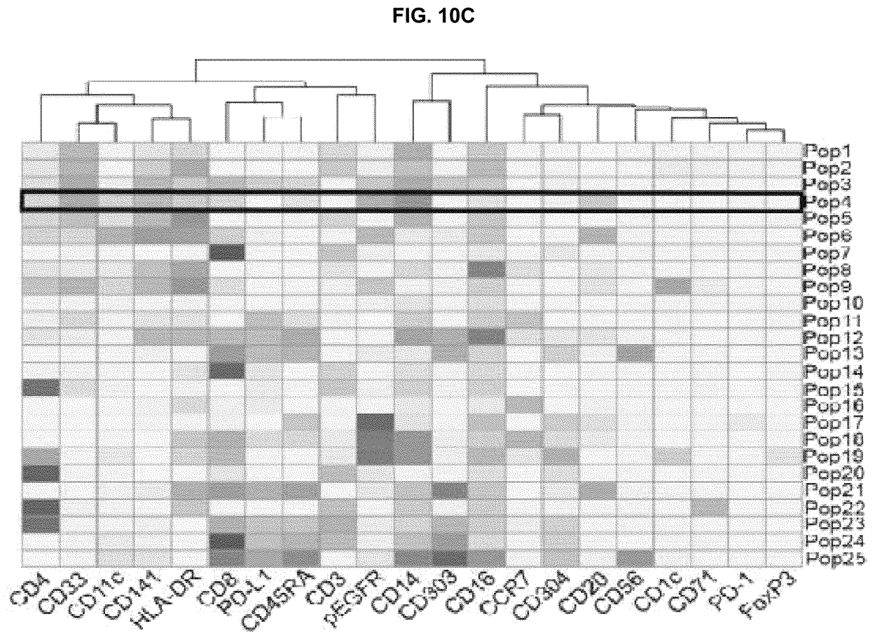

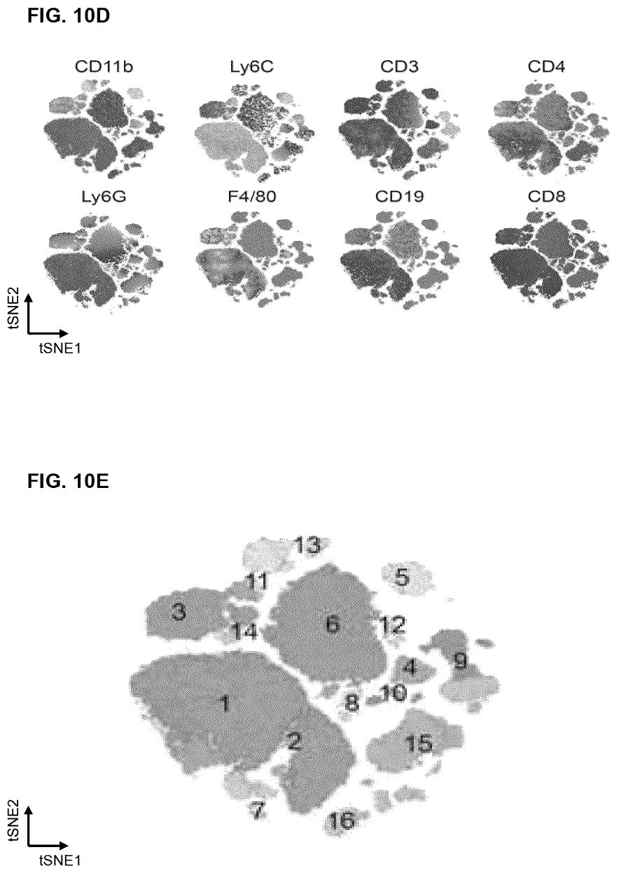

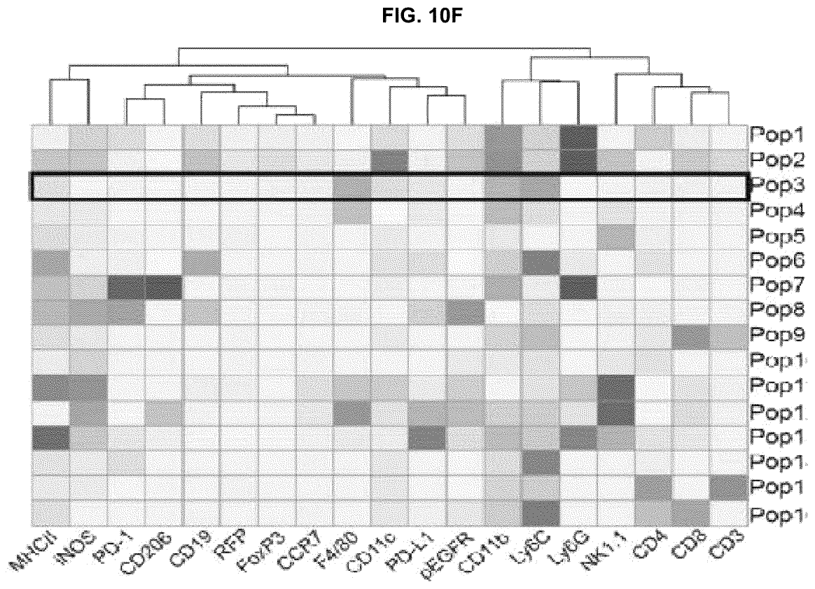

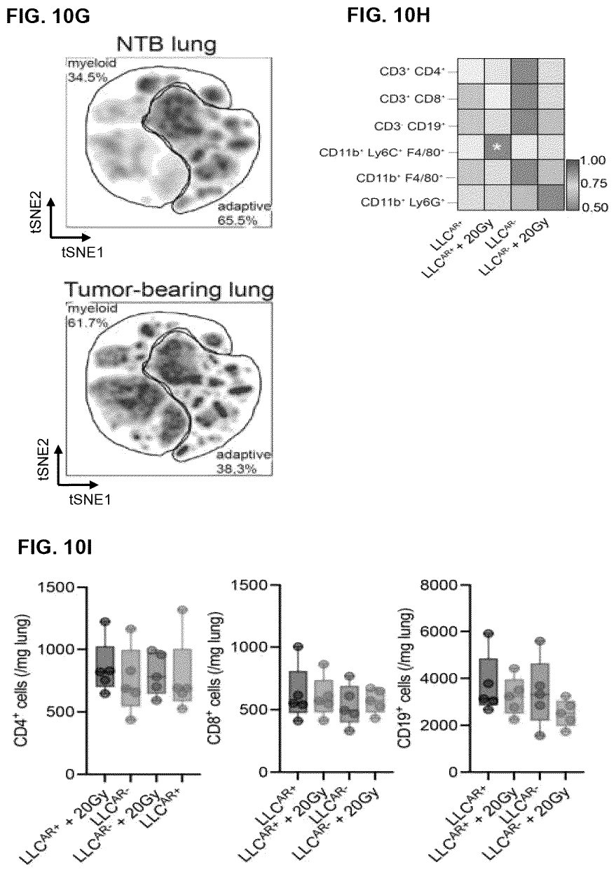

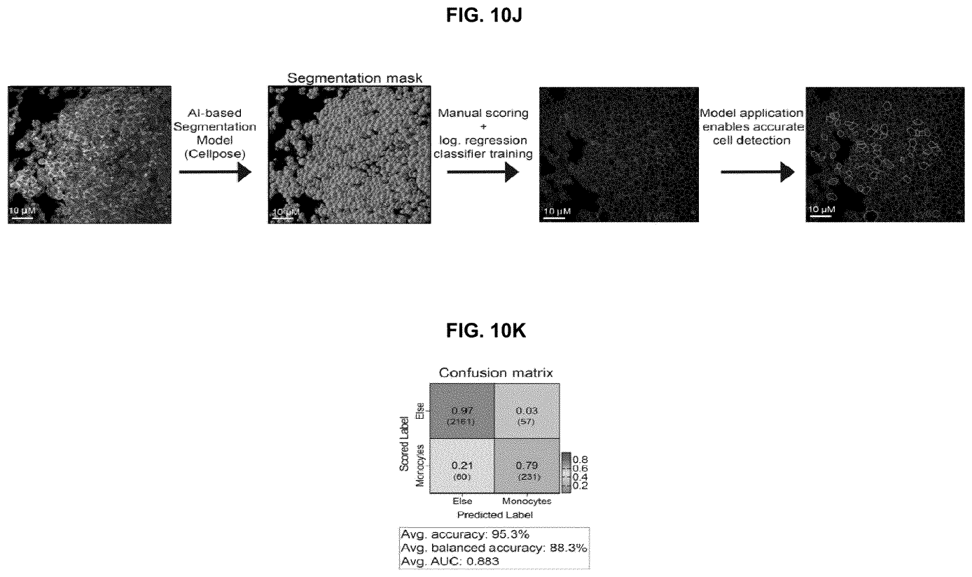

Given that the knock-out of Areg did not impact cellular proliferation or migration in vitro, we hypothesized that the host immune response contributed to the AREG-dependent metastatic growth observed in patients and murine models. AREG induces EGFR phosphorylation at tyrosine residue 992 (Tyr992), previously implicated in reduced immunoreactivity to esophageal carcinomas^{27,28}. We utilized spectral flow cytometry to validate the presence of Tyr992 pEGFR⁺ immune cells in peripheral blood mononuclear cells (PBMC) of metastatic non-small cell lung cancer (NSCLC) patients treated with SBRT in a second clinical trial at our institution (NCT03223155)²⁹. We analyzed 30 matched pre- and post-SBRT PBMC samples and created a t-distributed stochastic neighbor embedding (t-SNE)³⁰ projection of 800,000 live CD45⁺ cells. The highest expression of Tyr992 pEGFR was found on monocytes (CD33⁺, CD14⁺, CD16⁻) and dendritic cells (CD11c⁺, CD141⁺, HLA-DR⁺) (FIGS. 2A-2C, FIGS. 10A-10C). The fraction of CD33⁺ CD14⁺ pEGFR⁺ monocytes increased after SBRT (FIG. 2D). Trichotomization of the NSCLC patient cohort based on the fold change of CD33⁺ CD14⁺ pEGFR⁺ monocytes demonstrated a significantly worse PFS in the patients with the greatest increase following SBRT (FIGS. 2E-2F).

Mirroring our findings in PBMCs, the cell types with the highest Tyr992 pEGFR expression in murine lung tissues were myeloid cells; CD11b⁺ Ly6G⁺ granulocytes and Ly6C⁺ F4/80⁺ monocyte-derived macrophages (Ly6C⁺ MNPs) (FIGS. 2G-21, FIGS. 10D-10F). The fraction of myeloid cells significantly increased in the lungs of tumor-bearing mice compared to NTB mice (FIG. 10G). While RT resulted in a similar increase of Ly6G⁺ granulocytes in the lungs of LLC^{AR+} and LLC^{AR-} tumor-bearing mice, the fraction of pEGFR⁺ Ly6C⁺ MNPs exhibited a unique increase after RT in LLC^{AR+}, but failed to increase in the lungs of LLC^{AR-} tumor-bearing mice post-RT (FIG. 2J, FIG. 10H). These results suggested a possible link between Ly6C⁺ MNPs, RT-induced AREG and the increased metastasis size observed in mice bearing LLC^{AR+} tumors treated with 20 Gy. Similar patterns were not observed in CD4⁺ and CD8⁺ T cells or CD19⁺ B cells (FIGS. 10H-10I). We verified the presence of pEGFR⁺ Ly6C⁺ MNPs in the lung metastatic microenvironment by confocal microscopy (FIG. 2K) and developed an AI-based cell segmentation model, which detected a significant increase of MNPs post-RT (FIG. 2L, FIGS. 10J-10K). These results demonstrate that (i) Ly6C⁺ MNPs have the highest expression levels of Tyr992 pEGFR in PBMC of NSCLC patients and murine lung immune cells and (ii) an increase of pEGFR⁺ Ly6C⁺ MNPs in the lung metastatic microenvironment correlates with RT-induced AREG secretion.

### Example 3: scRNA-Seq Identifies AREG-Dependent Changes in Mononuclear Phagocytes in Lung Tissue from LLC^{AR+} and LLC^{AR-} Tumor-Bearing Mice Post-RT

Since MNPs were identified as target populations of tumor-secreted, RT-induced AREG and circulating Ly6C⁺ monocytes are known to differentiate into metastasis-associated macrophages^{31,32}, we investigated their involvement in AREG-dependent lung metastasis growth by single-cell RNA sequencing (scRNA-seq) of immune cells from the lungs of mice bearing LLC^{AR+} and LLC^{AR-} flank tumors five days post-IR.

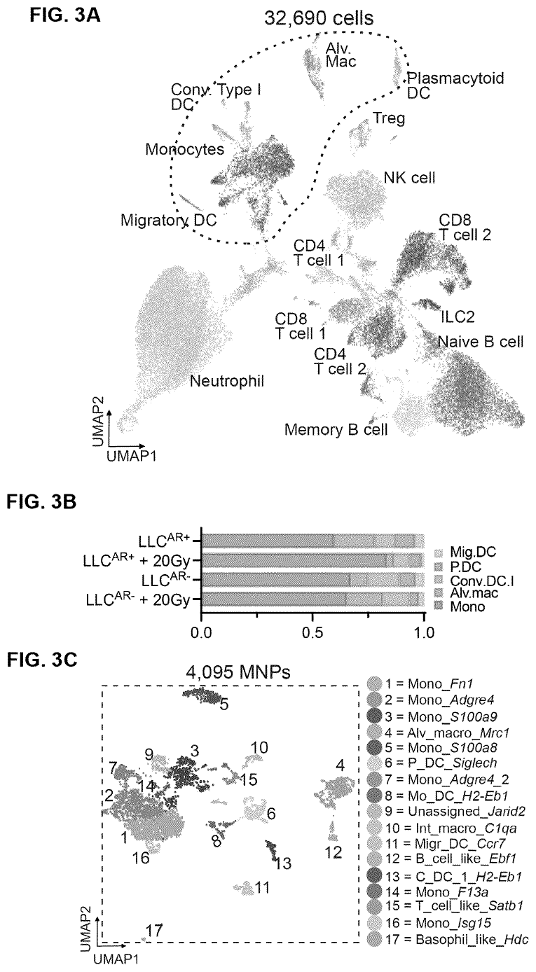

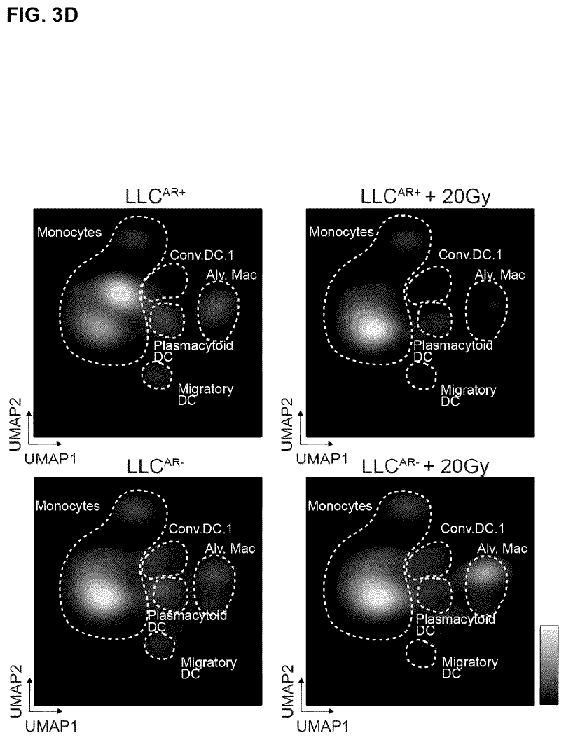

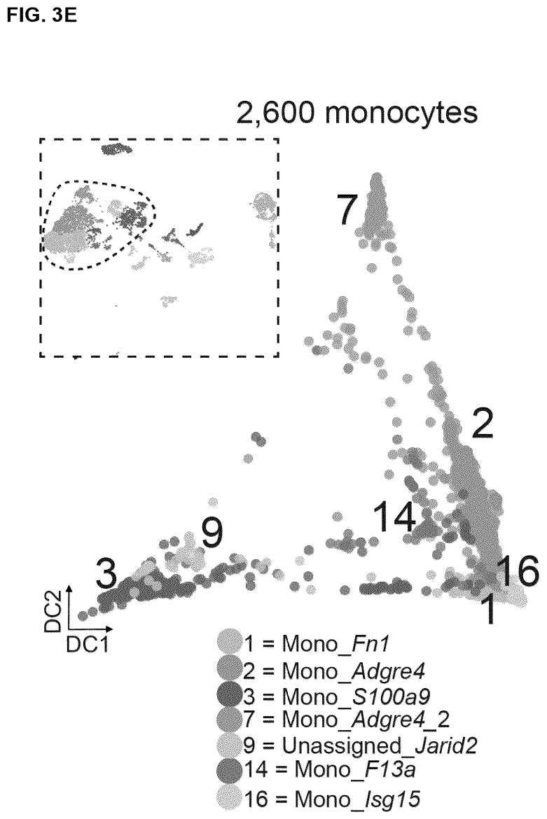

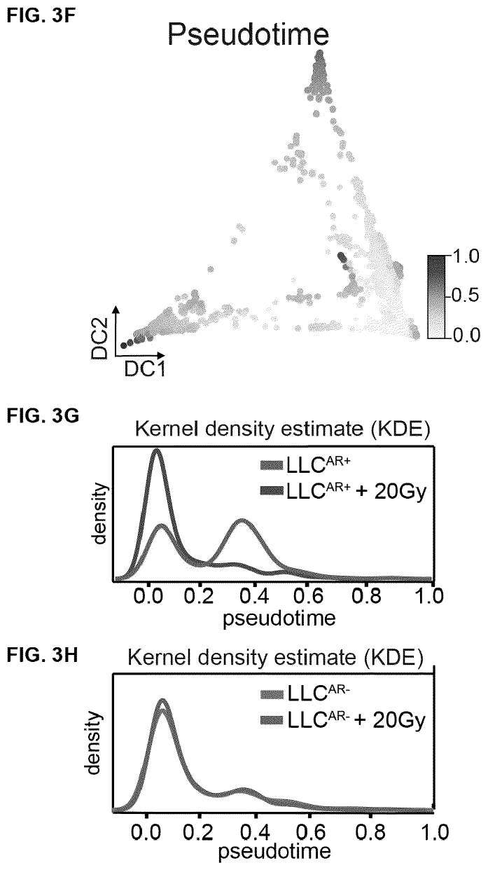

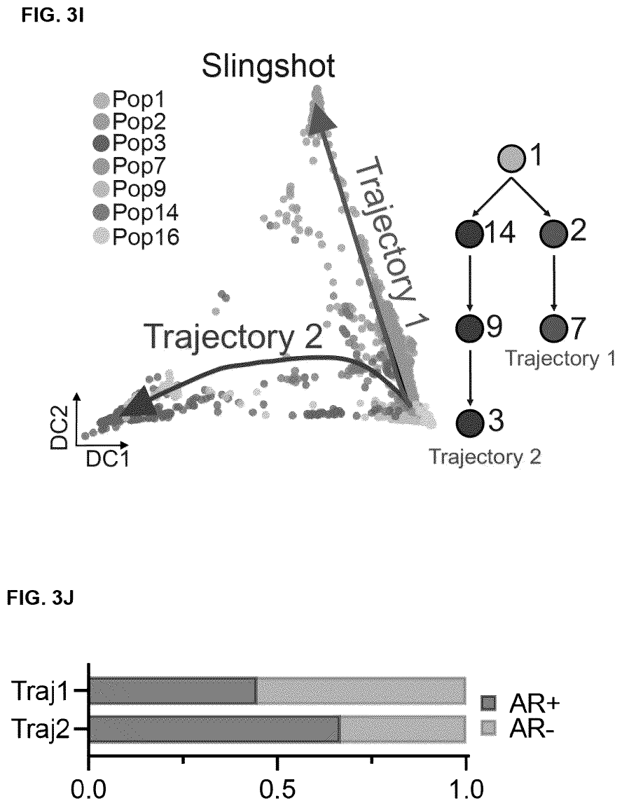

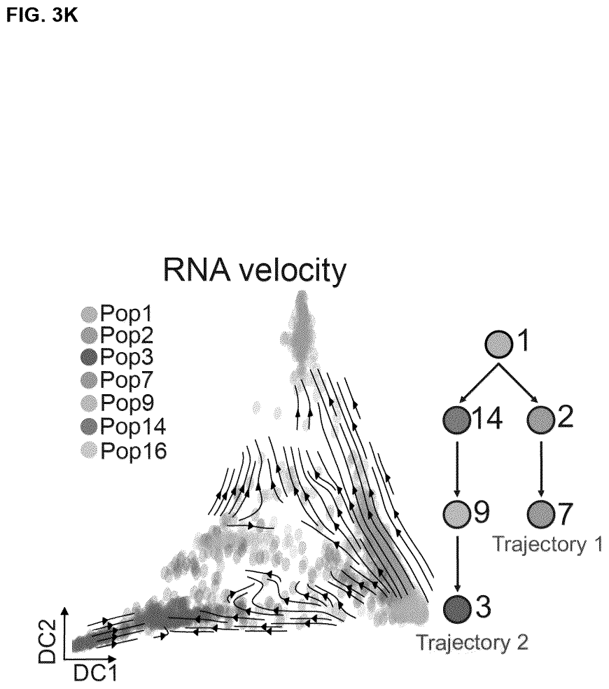

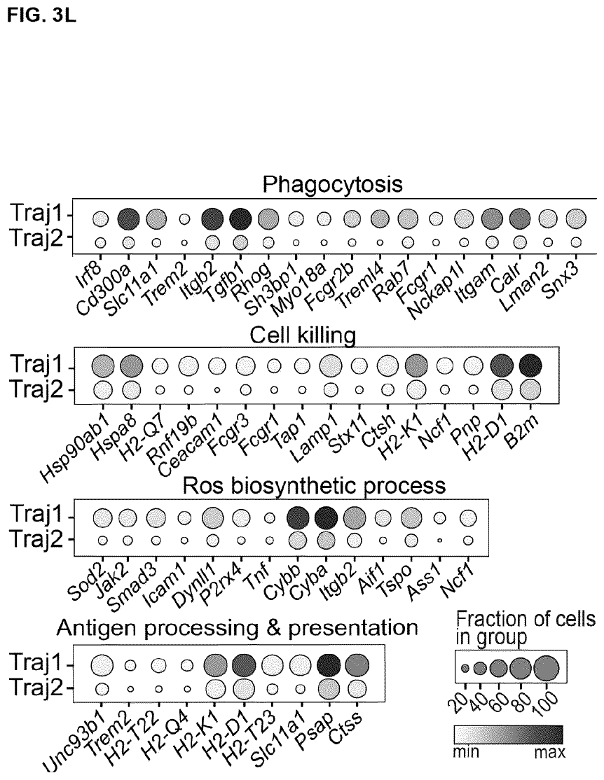

Unsupervised clustering of 32,690 CD45⁺ cells identified 21 clusters from 11 major cell lineages (FIG. 3A, FIGS. 11A-11D). We focused on the five clusters containing 4,095 MNPs (monocytes, alveolar macrophages, conventional type I DCs, plasmacytoid DCs, and migratory DCs) (FIG. 3A). Consistent with our previous results, local irradiation of LLC^{AR+} flank tumors increased the fraction of monocytes in the lung, which was not recapitulated under AR-conditions (LLC^{AR-} and LLC^{AR-}+20 Gy) (FIG. 3B). Comparing the gene expression of the LLC^{AR+}+20 Gy monocyte cluster to all other conditions, immunosuppressive genes (Lrg1, Retnlg, Eno1) and S100 proteins (S100a9, S100a8) were upregulated and genes involved in inflammation (Cd36, Dock2, Aff3), antigen processing and presentation (H2-K1, H2-Aa, Cd83), as well as phagolysosomal processes (Dock10, Picalm, Satb1) were downregulated (FIG. 11E). In contrast, we observed an opposite pattern in the LLC^{AR-}+20 Gy monocyte cluster, in which S100 and immunosuppressive genes (Cd274, Retnlg, Fosl1, Eif1) were downregulated and genes involved in inflammatory (Cd86, Tnfaip8) and phagolysosomal (Hexb, Cops9) processes were significantly upregulated (FIG. 11F).

Subclustering of MNPs identified 17 populations (FIG. 3C) and a distinctive change in the composition of the monocyte clusters post-IR under AR+ conditions (LLC^{AR+} and LLC^{AR+}+20 Gy), which was not observed under the AR-conditions (FIG. 3D and FIGS. 11G-11H). Monocytes from LLC^{AR+} were dominated by population 3 (Mono_S100a9), a population characterized by high expression of S100a9, S100a8 and Satb1. The most abundant cluster in LLC^{AR+}+20 Gy was population 1, also characterized by high expression of S100, immunosuppressive (Chil3, Hp, Gas7, Anxa1) and tissue-repair genes (Fn1, F13a1, Thbs1, Vcan), as well as a significant downregulation of genes involved in monocyte to macrophage differentiation (Pparg, Itgax, Ly75, Hsp90ab1) (FIGS. 11G-11I). MNPs from the lungs of AR-tumor-bearing mice showed a more diverse spread between all clusters and a larger fraction of clusters 4 (Alv_macro_Mrc1), 10 (Int_Macro_C1qa), and 13 (Class_DC_1_H2-Eb1) (FIG. 3D and FIGS. 11G-11H).

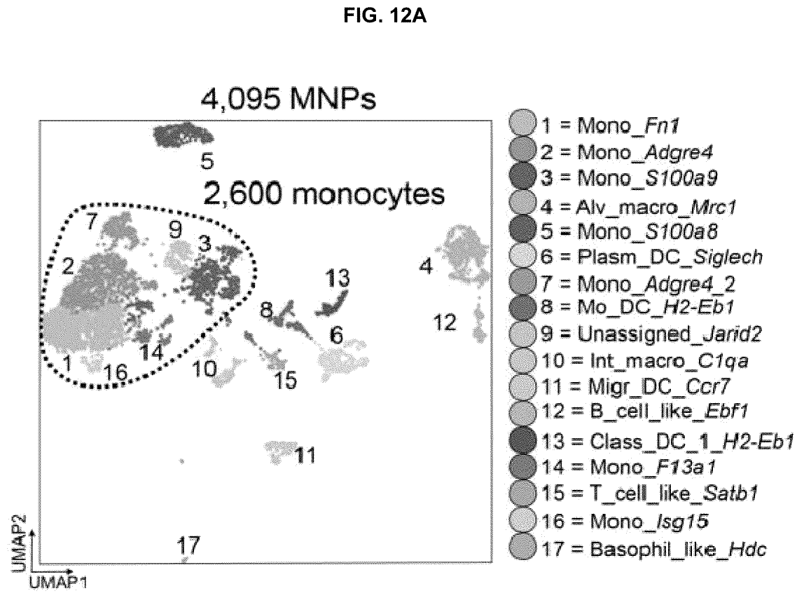

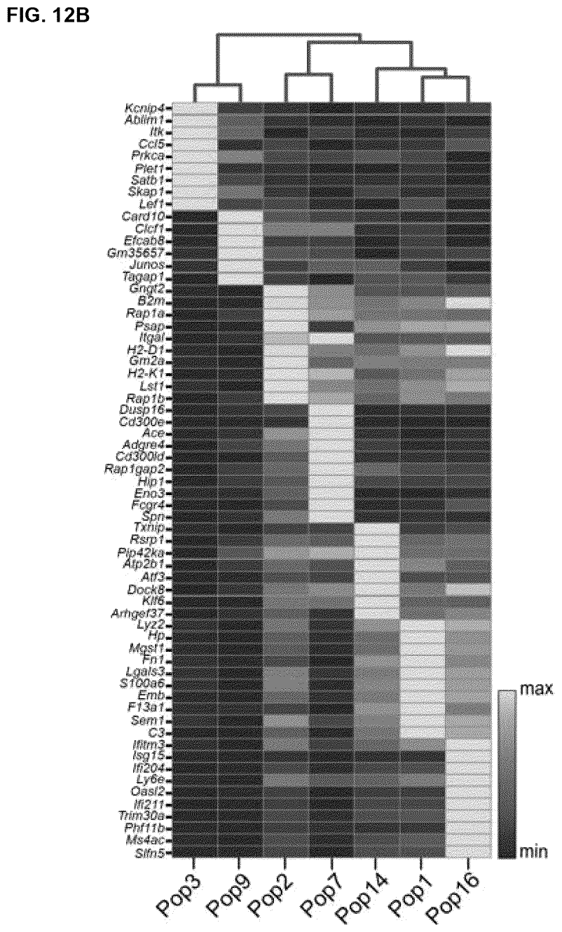

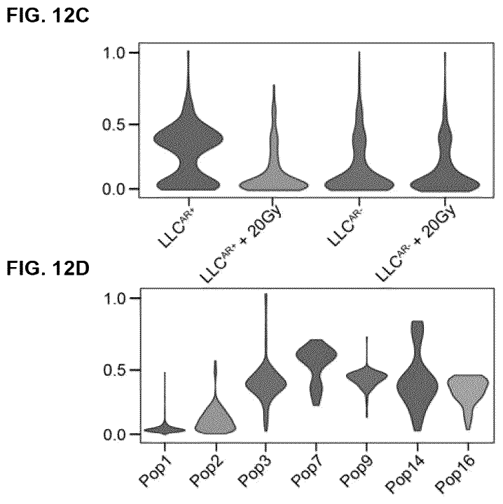

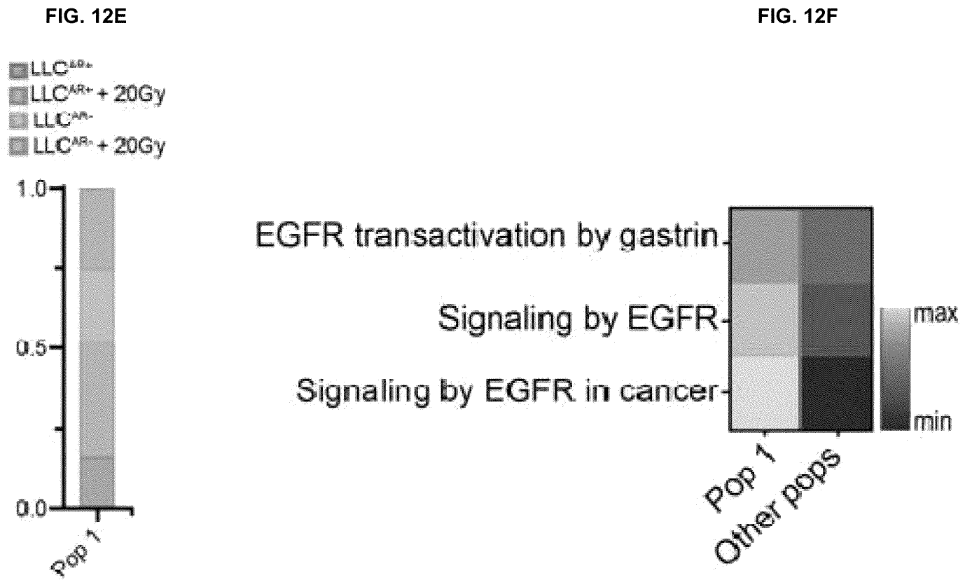

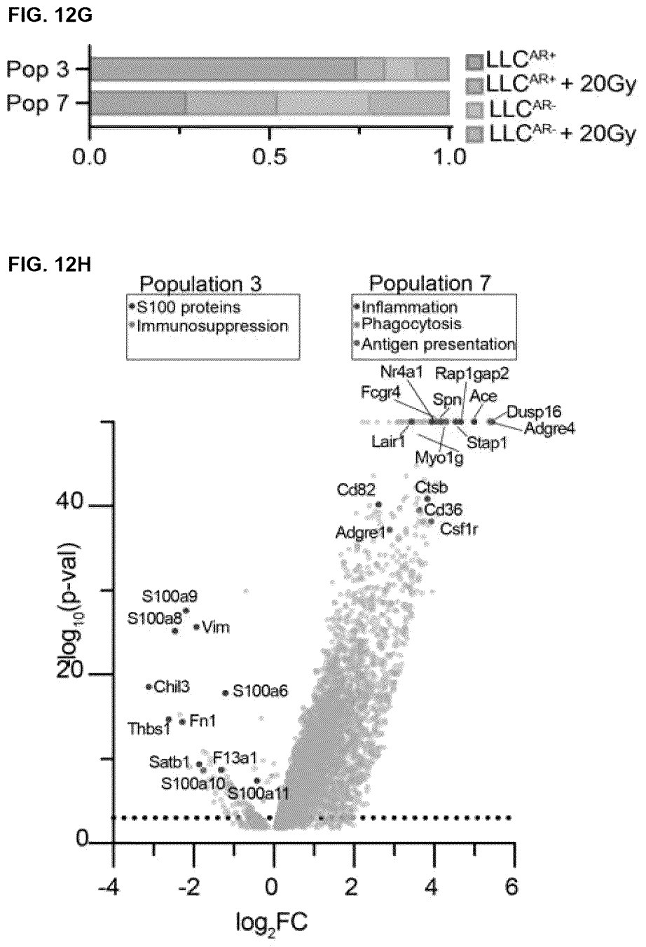

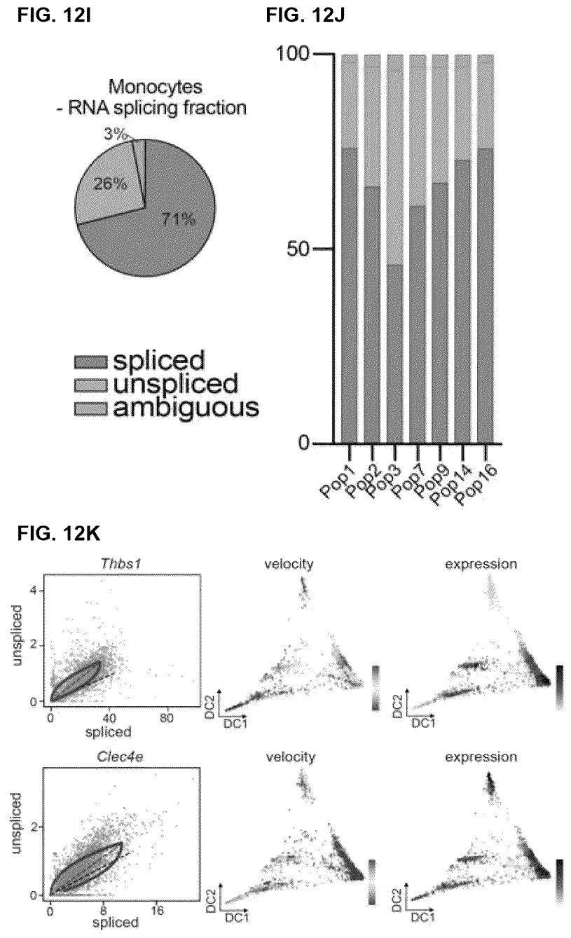

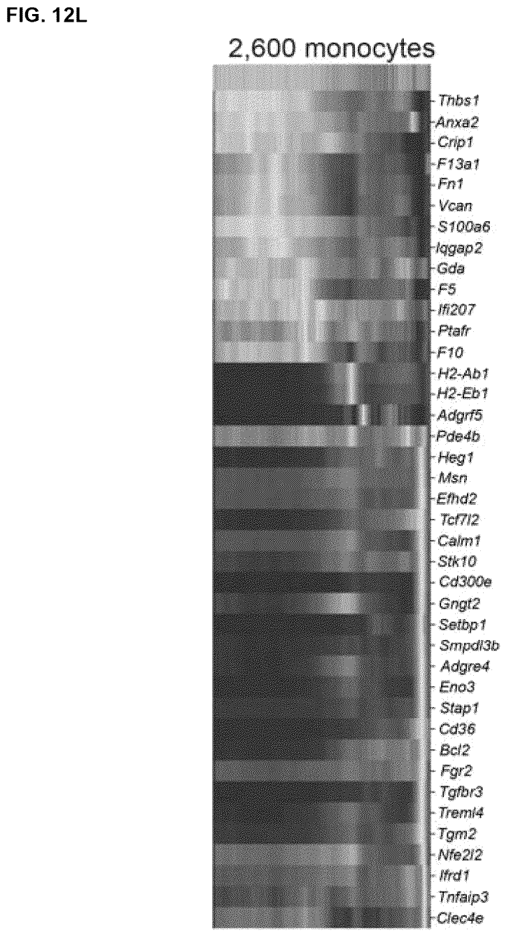

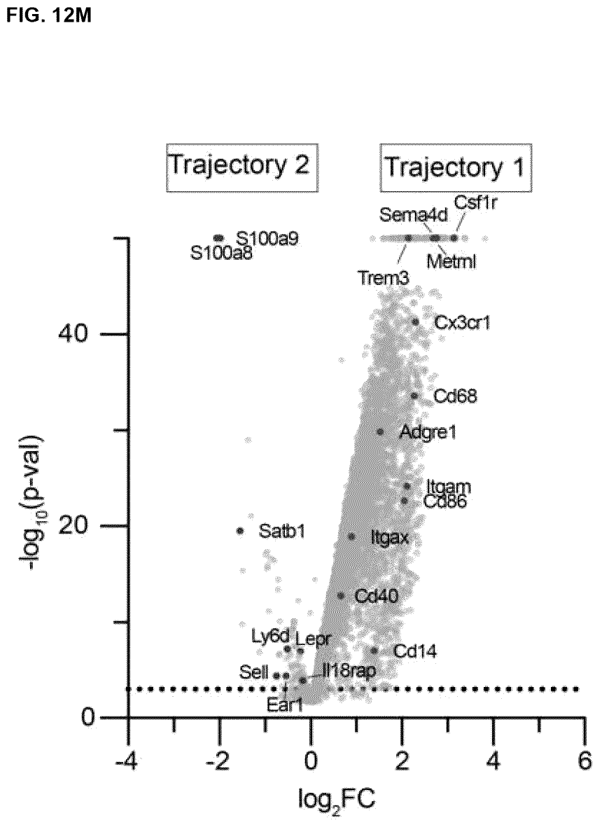

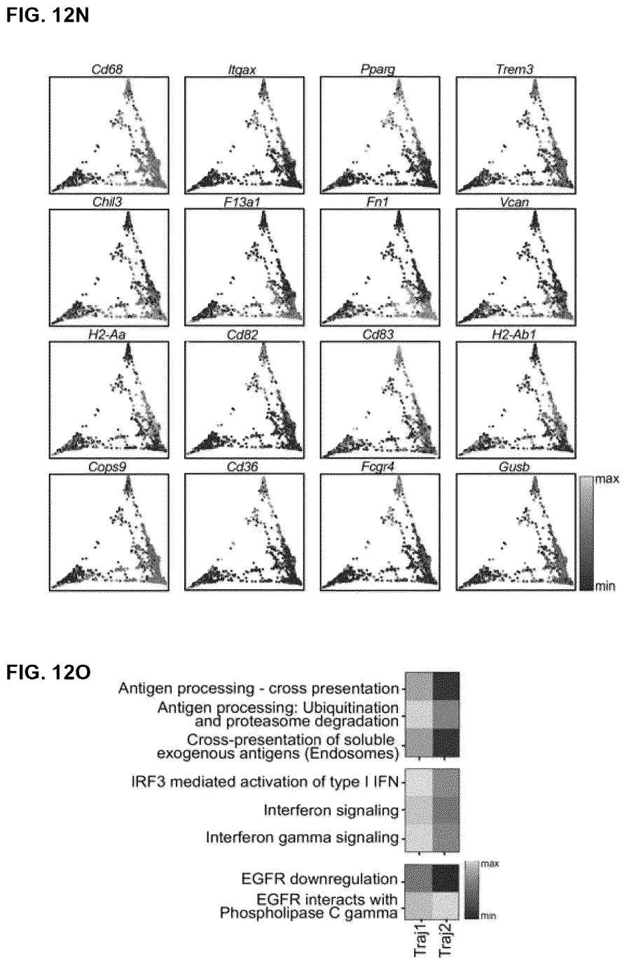

To improve our understanding how tumor-derived AREG affects functionally relevant monocyte differentiation trajectories, we inferred the development of monocyte subclusters by computing a diffusion map and ordering them along a pseudotime axis (FIGS. 3E-3F and FIGS. 12A-12B). Notably, the comparison of pseudotime states under AR+ conditions showed that a considerable fraction of monocytes from LLC^{AR+}+20 Gy were arrested at an earlier pseudotime state and less likely to differentiate to later stages (FIG. 3G, FIG. 12C). In contrast, cells from AR-conditions were more likely to differentiate, measured by progression along pseudotime, and local tumor irradiation did not affect their pseudotime density (FIG. 3H, FIG. 12C). Corresponding to the differentiation arrest, we found that the largest fraction of cells in population 1—the population with the lowest pseudotime—was derived from LLC^{AR+}+20 Gy (FIG. 12E). Notably, Pop1_Fn1 also showed a higher expression of EGFR signaling pathways compared with other subpopulations (FIG. 12F).

Trajectory inference using Slingshot with population 1 as a starting population predicted two main trajectories: trajectory 1 developed through population 2 to 7. An alternative trajectory, trajectory 2, instead moved through populations 14 and 9, ending in population 3 (FIG. 3I). Population 7 was made up of equal parts of cells from all conditions and characterized by high expression of genes involved in inflammatory, phagocytic and antigen processing functions such as Fcgr4, Myolg, and Ctsb. In contrast, population 3 was dominated by monocytes from AR+ conditions and characterized by high expression of immunosuppressive genes (Chil3, Thbs1, Fn1, F13a1) and genes encoding S100 proteins (FIGS. 12G-12H). Correspondingly, trajectory 1 was followed equally by cells from AR+ and AR-conditions, while a larger fraction of trajectory 2 was made up of cells from AR+ conditions (FIG. 3J). These findings were reproduced using RNA velocity analysis (FIG. 3K and FIGS. 12I-12L).

Cells following trajectory 1 increasingly expressed macrophage differentiation and function genes along their differentiation trajectory (Cd68, Trem3, Csflr, Adgre1), and while MNPs in trajectory 2 showed a global downregulation of gene expression, they significantly upregulated genes linked to immunosuppression (S100a8, S100a9, Sell, Il18rap) (FIGS. 12M-12N). Further examination of previously published gene signatures involved in anti-tumor functions of monocyte-derived macrophages³³ revealed their significant downregulation in trajectory 2 (FIG. 3L), correlating with higher EGFR signaling (FIG. 12O). We concluded that the primary route of monocyte differentiation occurred along trajectory 1 and encompassed increasing expression of macrophage markers and gene signatures associated with phagocytosis, antigen processing and inflammatory response. By contrast, in the response to tumor-derived AREG, monocytes were more likely to be redirected along an alternative trajectory (trajectory 2), characterized by decreased anti-tumor functions and increased immunosuppression.

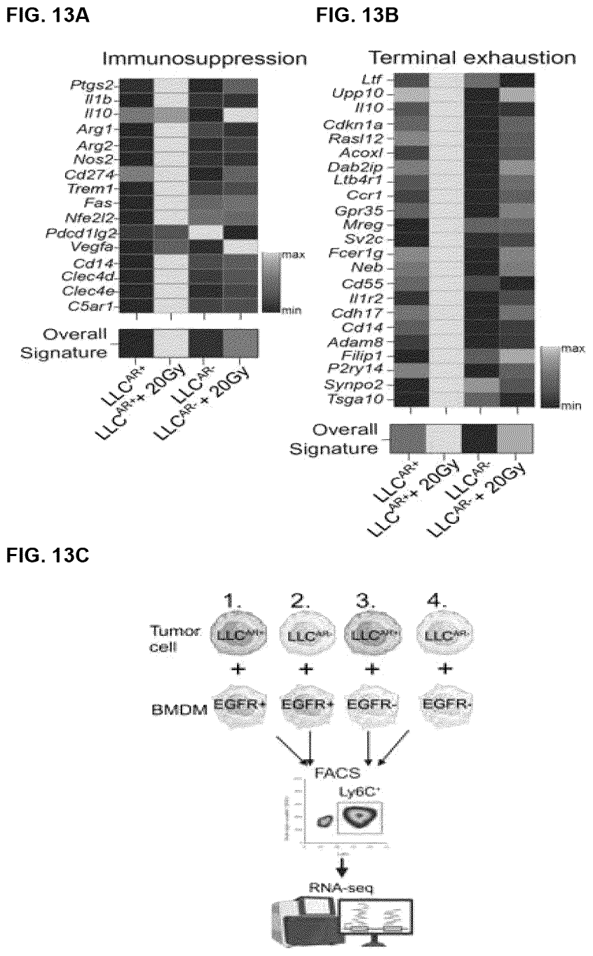

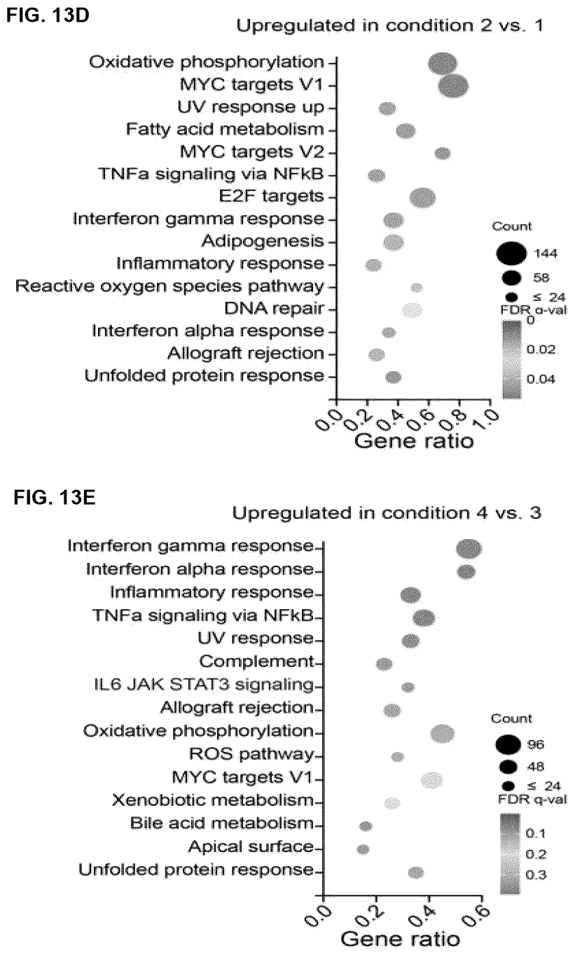

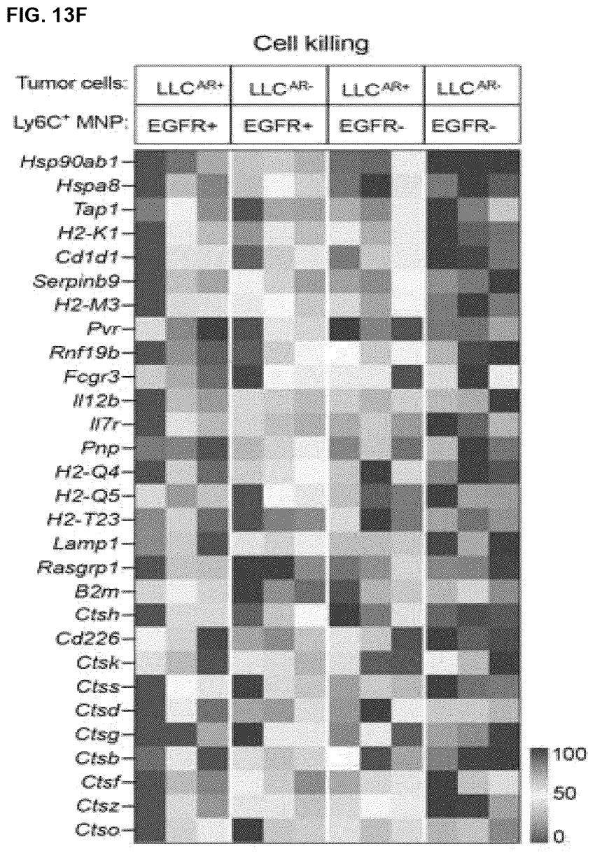

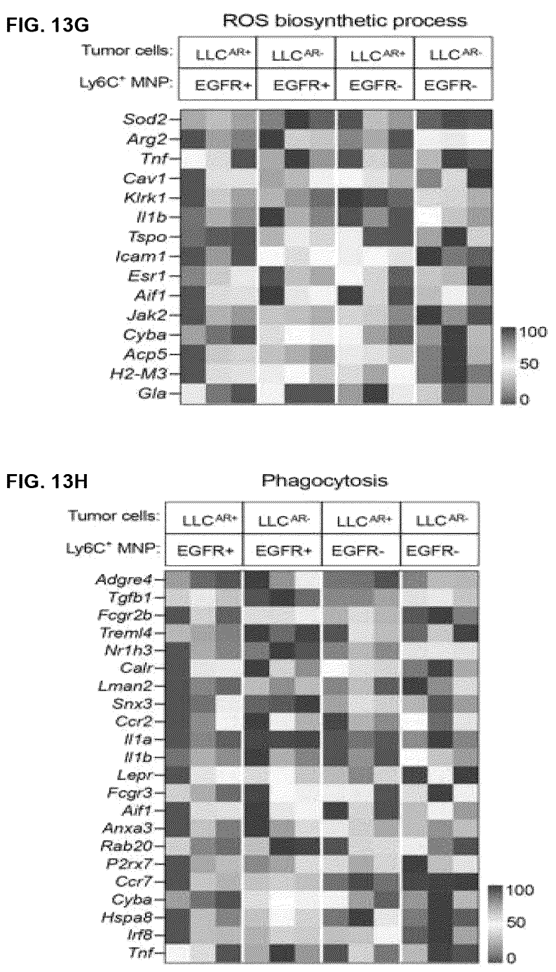

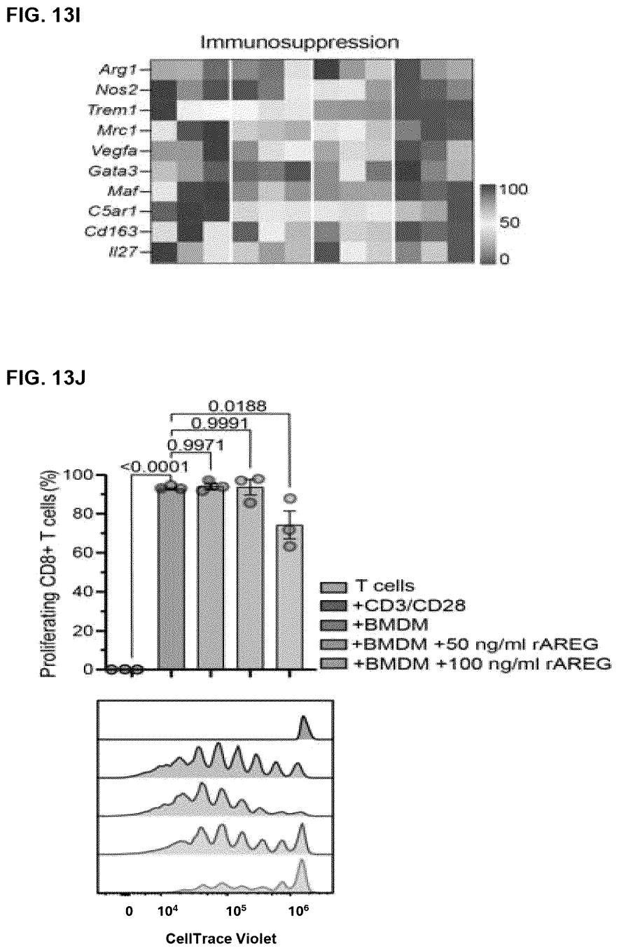

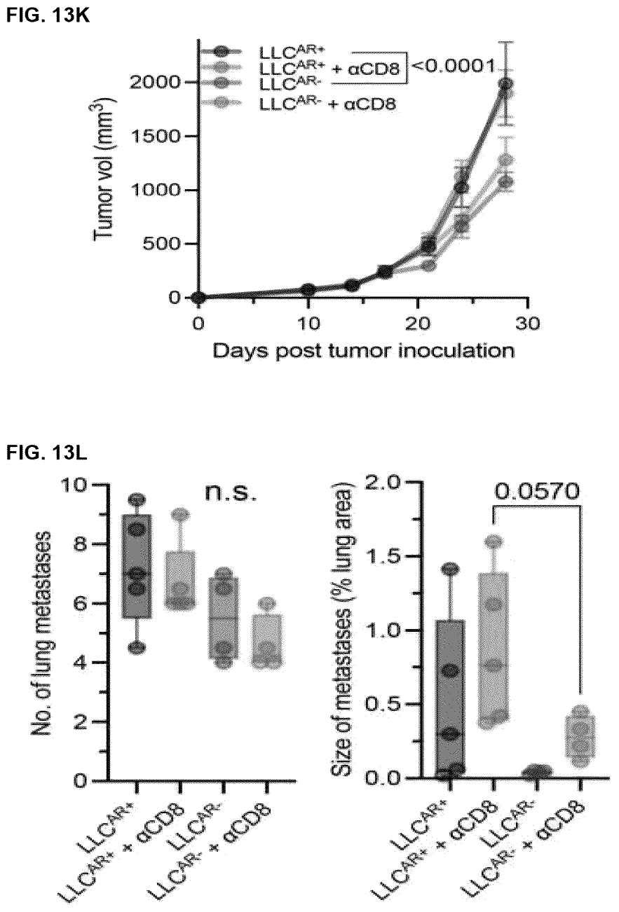

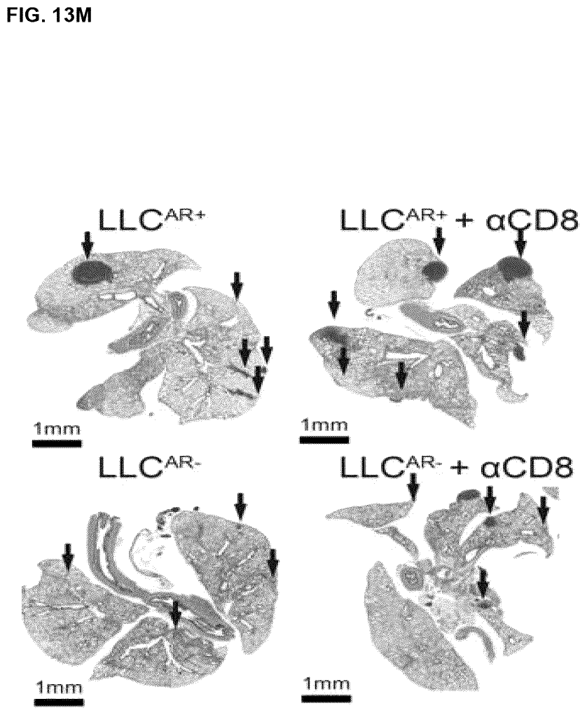

The known immunosuppressive functions of monocytes after IR³⁴ and their increased presence in the lungs of LLC^{AR+}+20 Gy mice suggested that suppression of the adaptive antitumor T-cell response may enable metastatic proliferation under AR+ conditions. In support of this, we observed an increased expression of immunosuppressive gene in LLC^{AR+}+20 Gy monocytes (FIG. 13A) and correspondingly, a terminally exhausted gene signature of CD8⁺ T cells, indicative of a decreased proliferative and cytokine production capacity³⁵ (FIG. 13B). RNA-seq of Ly6C⁺ MNPs from EGFR+ (Egfr^{flx/flx}) and conditionally EGFR-deficient (LysM^{ΔEgfr}, EGFR−) mice co-cultured with LLC^{AR+} and LLC^{AR-} tumor cells (FIG. 13C) revealed that tumor cell AREG-expression induces an anti-inflammatory transcriptomic state in EGFR+ MNPs. Gene Set Enrichment Analysis (GSEA) identified upregulated pro-inflammatory pathways in EGFR+ MNPs co-cultured with LLC^{AR-} tumor cells (FIG. 13D), an effect further amplified in EGFR− MNPs, where interferon gamma/alpha responses, inflammatory response and TNFα signaling were the top upregulated pathways in the presence of LLC^{AR-} cells (FIG. 13E). Gene signatures related to phagocytosis, cell killing and ROS biosynthesis³³ showed highest expression in EGFR− MNPs co-cultured with LLC^{AR-} tumor cells (FIGS. 13F-13H). In contrast, genes associated with immunosuppression were most highly expressed in EGFR+ MNPs co-cultured with LLC^{AR+} tumor cells (FIG. 13G). AREG further induced a T-cell suppressive phenotype in bone-marrow-derived monocytes (BMDM) (FIG. 13J).

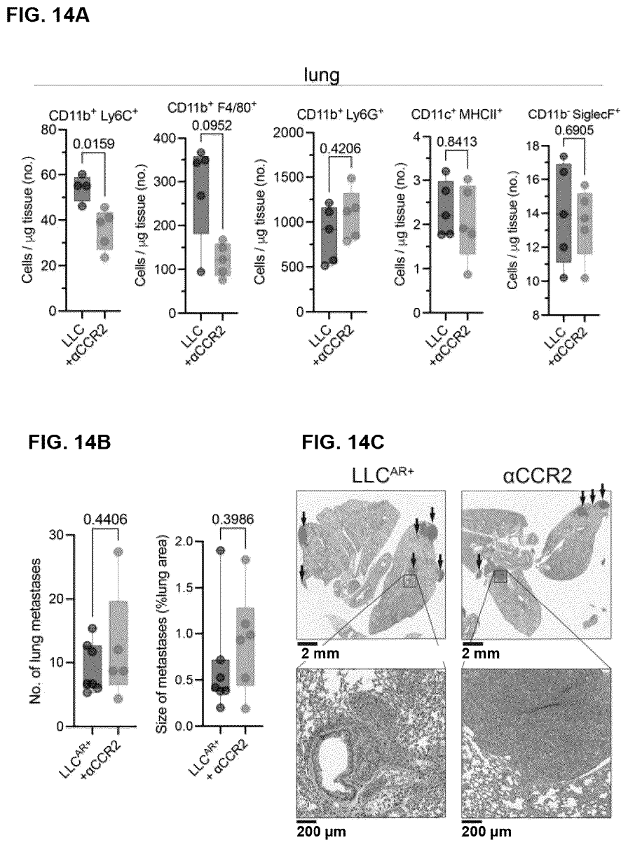

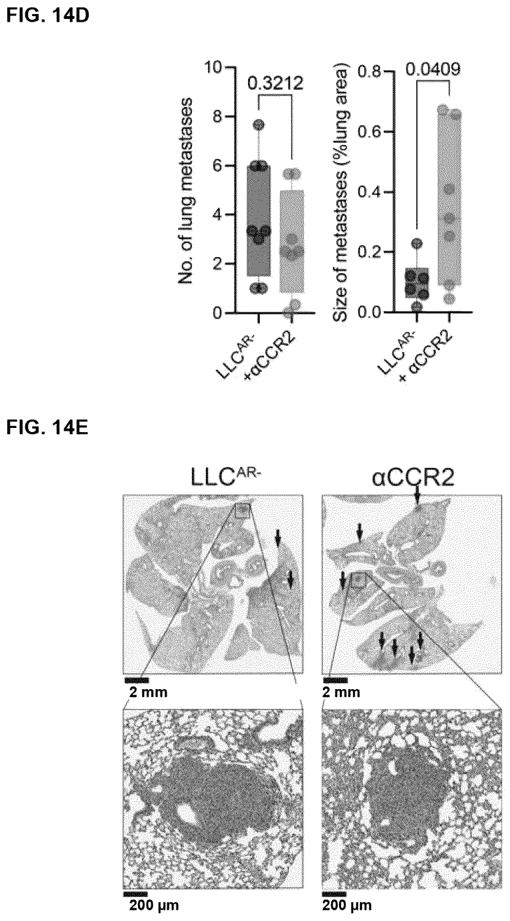

Despite these findings, antibody-mediated depletion of CD8⁺ T cells did not eliminate the size difference between LLC^{AR+} and LLC^{AR-} metastases (FIG. 13k-m), suggesting that CD8⁺ T cells are not the primary mediators of metastatic size differences in vivo. We hypothesized that Ly6C⁺ MNPs are key effector cells. CCR2 antibody³⁶-mediated depletion of Ly6C⁺ MNPs (FIG. 14A) did not alter lung metastasis size in LLC^{AR+} tumor-bearing mice, but significantly increased it in mice bearing LLC^{AR-} tumors (FIGS. 14B-14E), indicating that Ly6C⁺ MNPs play a crucial role in constraining LLC^{AR-} lung metastasis growth.

### Example 4: AREG Upregulates CD47 in Tumor Cells Resulting in SIRPα-Mediated MNP Phagocytosis Suppression

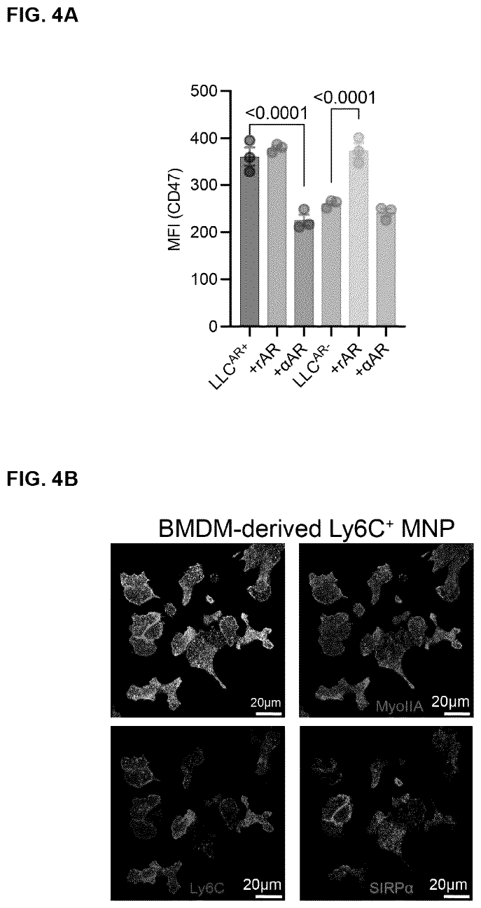

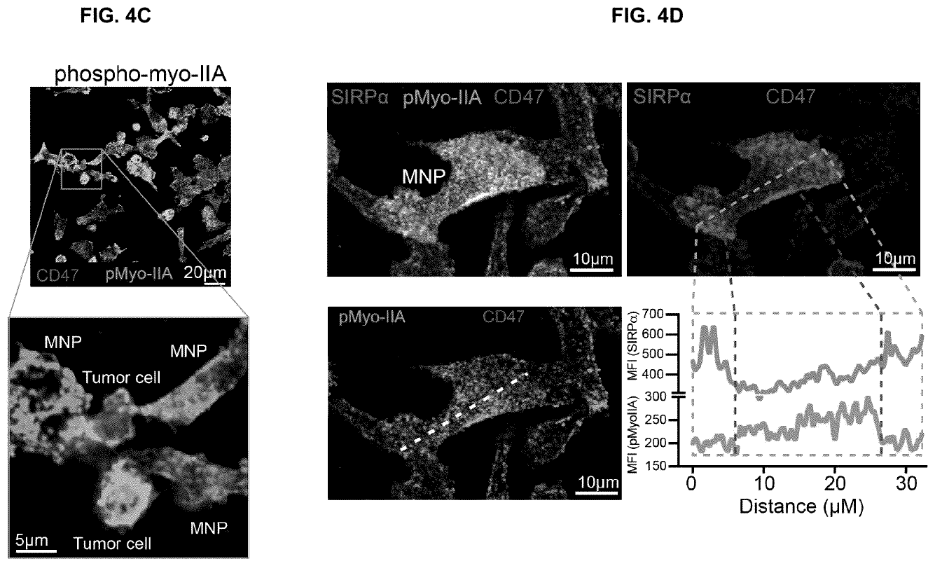

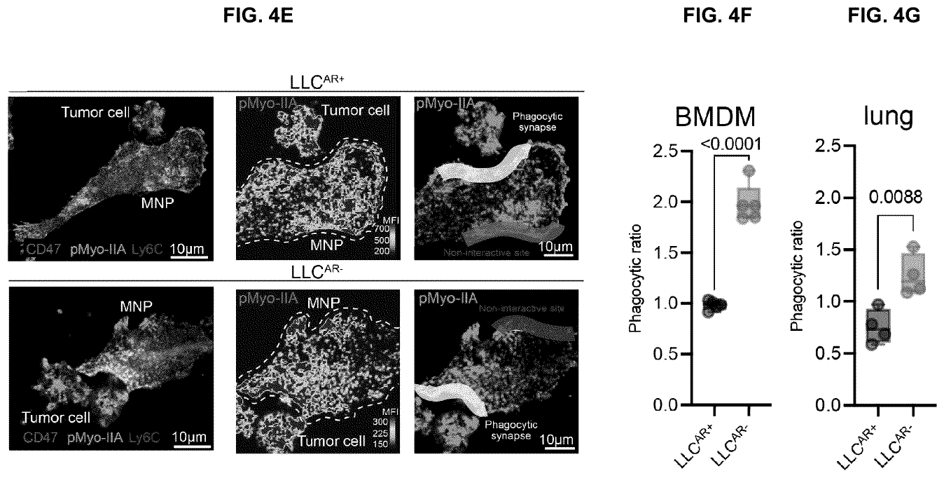

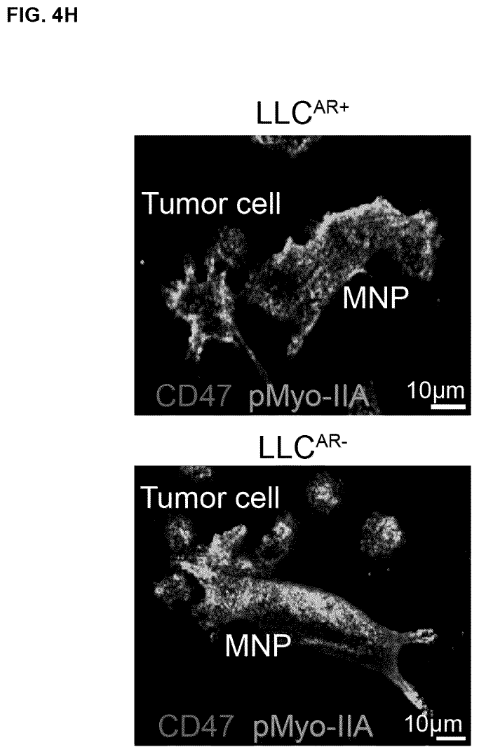

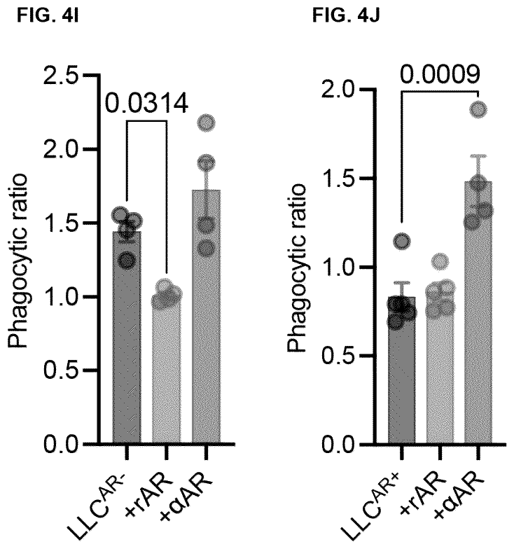

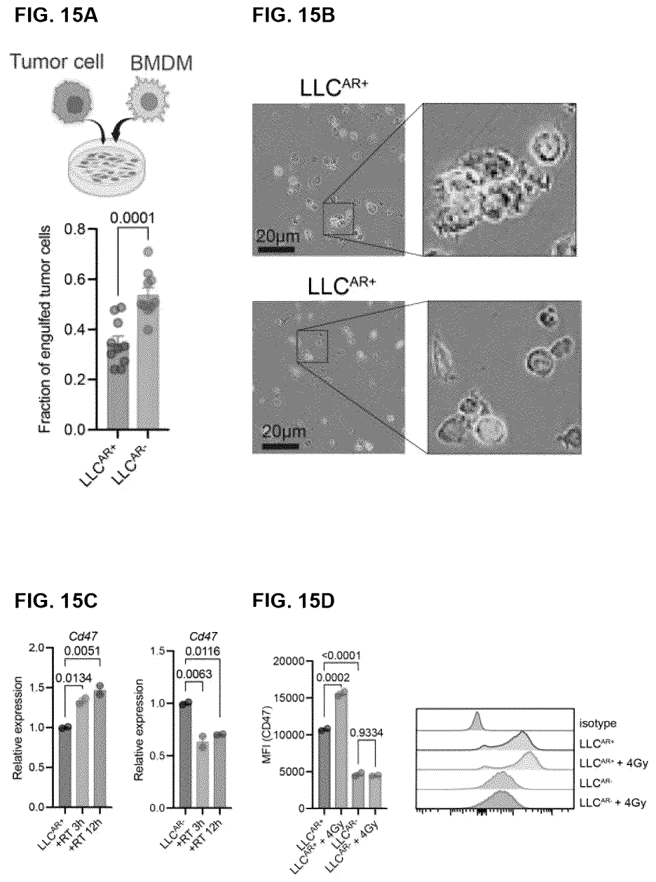

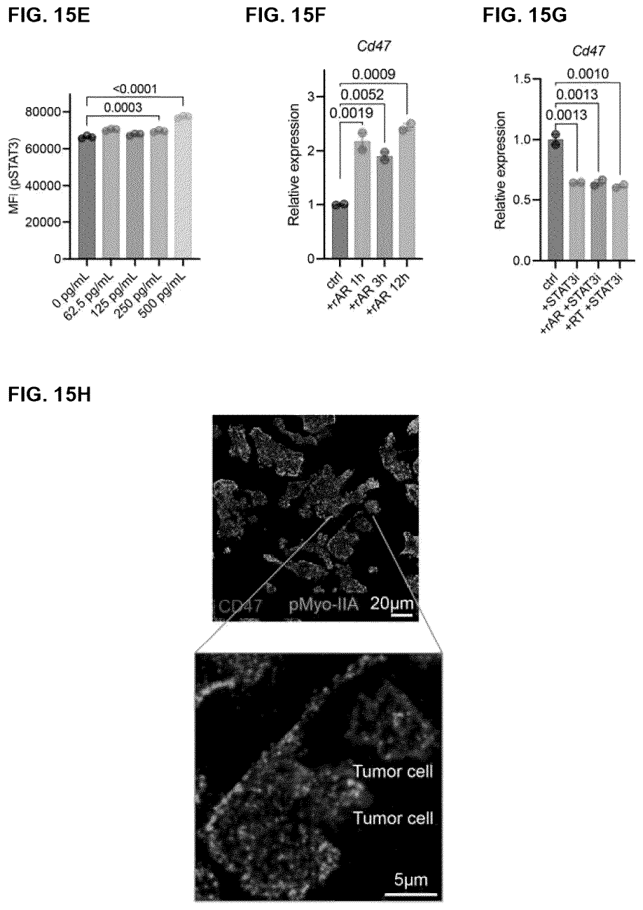

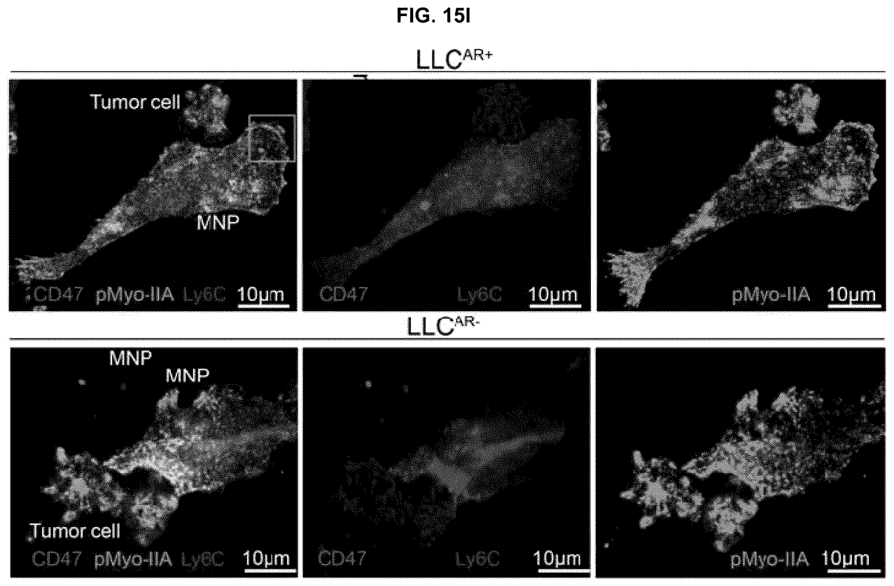

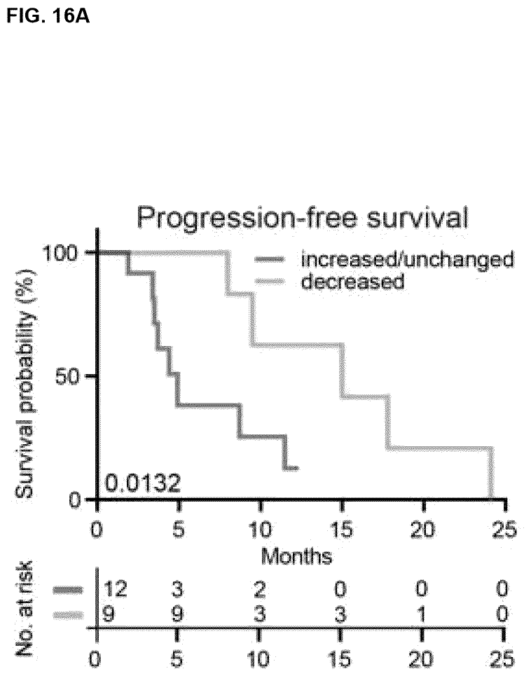

The phagocytic activity of MNPs plays a central role in tumor control^{33,37,38}, both in tissue-resident and monocyte-derived cells within the lung tumor microenvironment^{39,40}. AREG has been implicated in reducing phagocytosis-induced cell death⁴¹; however, the effects of tumor cell-derived AREG on the phagocytic capacity of MNPs remain unclear. To investigate this, we utilized a co-culture system and observed that BMDM-derived Ly6C⁺ MNP phagocytosed significantly fewer LLC^{AR+} tumor cells compared to LLC^{AR-} tumor cells (FIGS. 15A-15B). This reduced phagocytic activity correlated with a significantly higher expression of the “don't-eat-me” signal CD47 on LLC^{AR+}, as well as an RT-induced CD47 upregulation, which was absent in LLC^{AR-} tumor cells (FIGS. 15C-15D). Treatment of LLC^{AR-} tumor cells with recombinant AREG (rAR) increased CD47 protein levels, while treatment of LLC^{AR+} with an AREG-targeting antibody (αAR) decreased CD47 levels (FIG. 4A). AREG increases STAT3 phosphorylation in tumor cells^{42,43} (FIG. 15E) and STAT3 is known to upregulate CD47 by binding to consensus DNA elements in the promoter and intron regions of Cd47 in lung cancer cells⁴⁴. Correspondingly, treatment with a STAT3 inhibitor (Stattic, STAT3i⁴⁵) suppressed both rAR- and RT-induced Cd47 upregulation (FIGS. 15F-15G). These results suggest that RT-induced AREG upregulation promotes phagocytosis resistance of tumor cells by increasing STAT3-mediated CD47 expression.

CD47 inhibits MNP phagocytosis by SIRPα-SHP-1-mediated dephosphorylation of activated myosin-IIA (MyoIIA), reducing actomyosin contractility at the phagocytic synapse⁴⁶⁻⁴⁸. We used high-resolution confocal microscopy to assess Ly6C⁺ MNP phagocytic function, by visualizing MyoIIA, Ly6C and SIRPα (FIG. 4B). Ly6C⁺ MNPs co-cultured with LLC tumor cells showed no changes in unphosphorylated myo-IIA expression (FIG. 15H), but elevated phospho-myo-IIA-levels at phagocytic synapses (FIG. 4C). Notably, phospho-myo-IIA was reduced in cell-cell contact areas with high SIRPα-expression (FIG. 4D), indicating that decreased phospho-myo-IIA levels reflect CD47-SIRPα-mediated suppression of phagocytosis. We quantified this suppression using a “phagocytic ratio”: phospho-myo-IIA fluorescence intensity at the phagocytic synapse compared to a distant membrane section. A phagocytic ratio >1 indicated reduced CD47-SIRPα-mediated myo-IIA dephosphorylation and enhanced phagocytic activity. BMDM-derived Ly6C⁺ MNPs co-cultured with LLC^{AR-} cells had significantly higher phagocytic ratios than those co-cultured with LLC^{AR+} cells (FIGS. 4E-4F, FIG. 15I), a finding confirmed in Ly6C⁺ MNPs isolated from the lungs (FIGS. 4G-4H). The phagocytic ratio of MNPs co-cultured LLC^{AR-} cells was significantly decreased after tumor-cell treatment with rAR, while αAR-treatment of LLC^{AR+} cells enhanced the phagocytic ratio of co-cultured MNPs (FIGS. 41-4J). These findings demonstrate that AREG signaling upregulates CD47 on tumor cells, resulting in SIRPα-mediated myo-IIA-dephosphorylation and phagocytosis suppression in Ly6C⁺ MNPs.

### Example 5: AREG Blockade Reduces Metastasis Size in Combination with IR, EGFR TKI and Anti-CD47 Immunotherapy

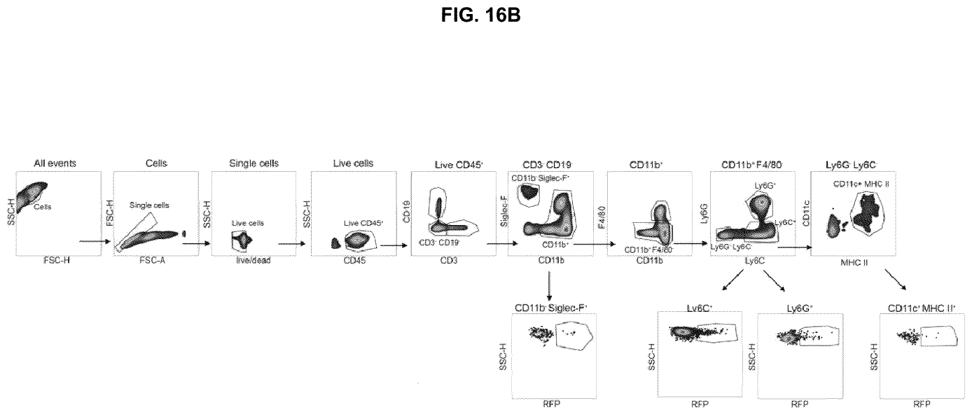

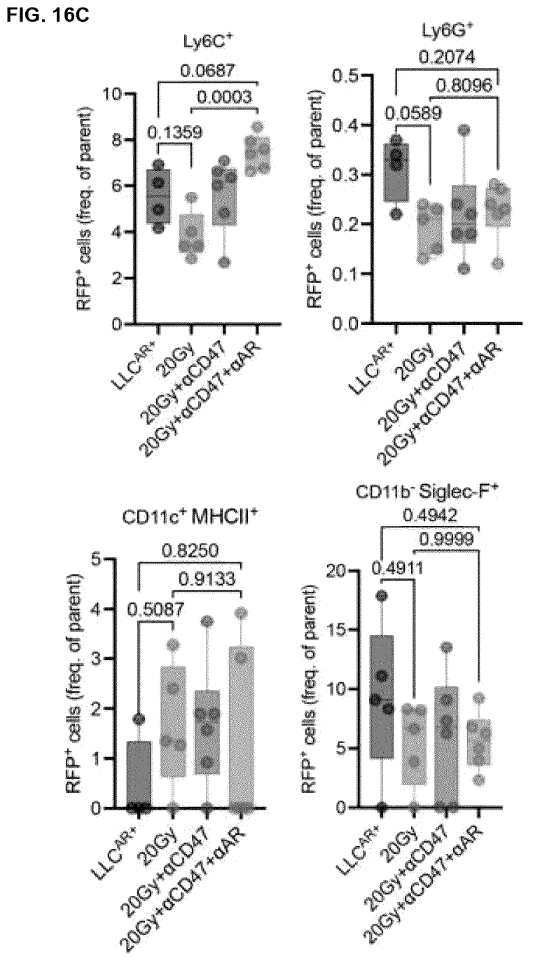

To evaluate the effects of chronic systemic AREG elevation in patients, we analyzed 42 matched-paired serum samples of 21 NSCLC patients prior to SBRT, as well as after SBRT and three cycles of ICB (from our institutional trial NCT03223155)²⁹. We found that patients with elevated serum AREG levels after SBRT+ICB had a significantly shorter PFS (FIG. 16A). Thus, we hypothesized that blocking systemic AREG may improve RT efficacy and delay tumor progression.

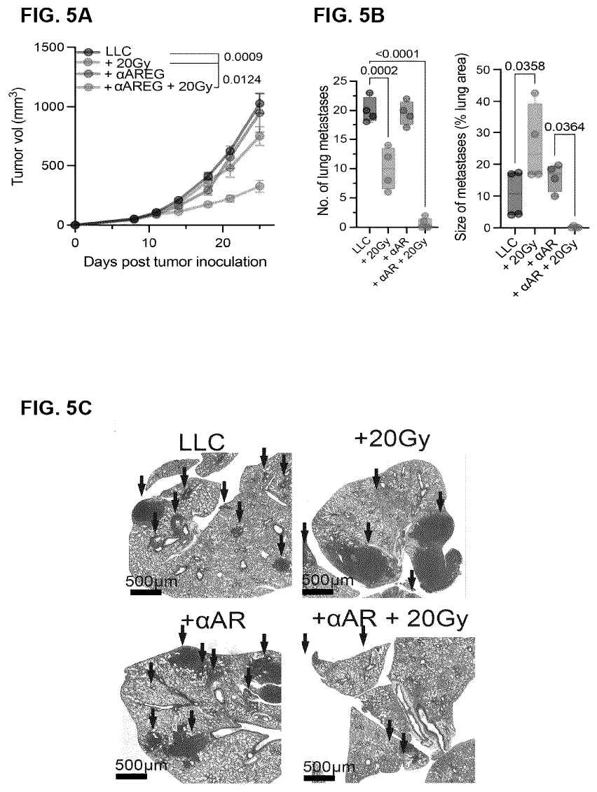

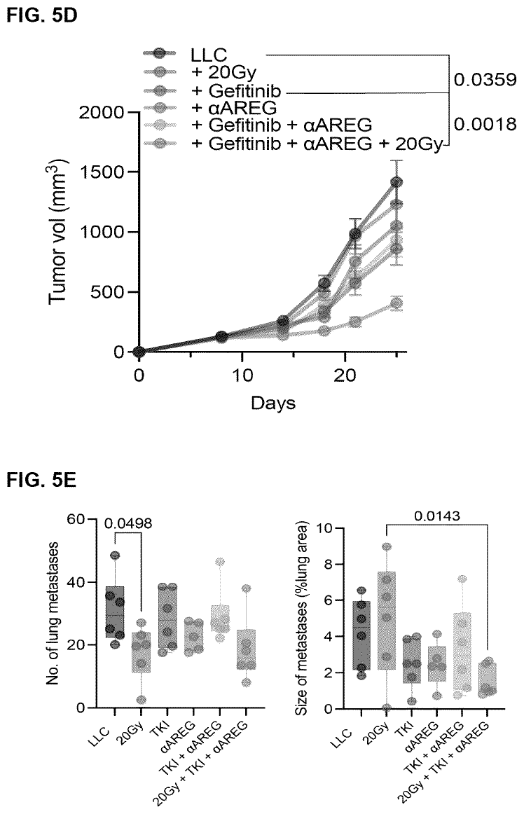

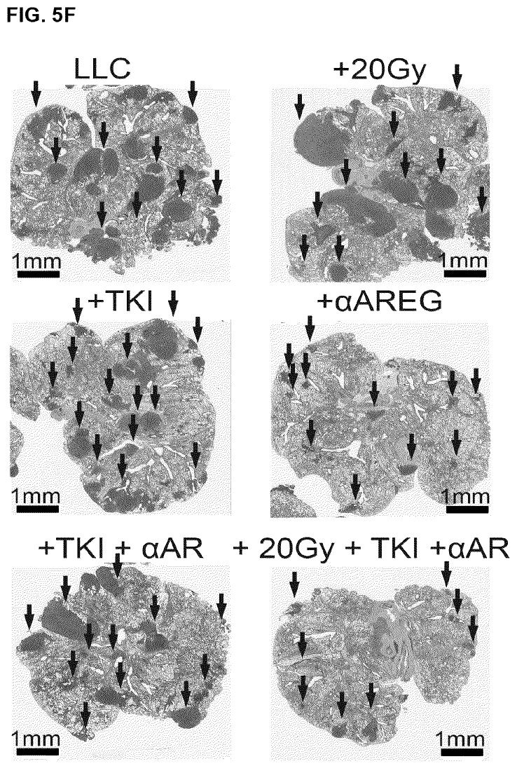

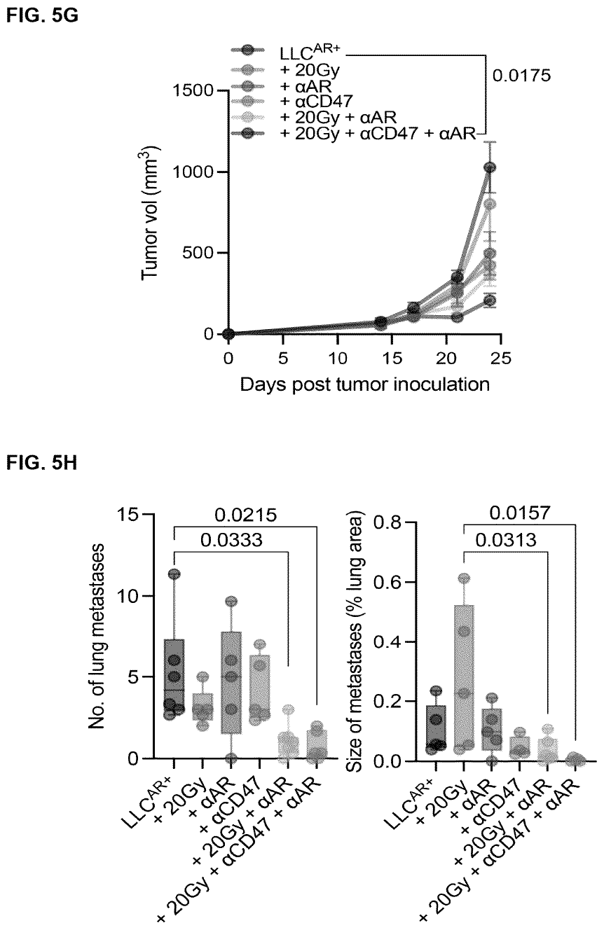

EGFR tyrosine kinase inhibition (TKI) has been tested in concurrent and sequential combination with RT in patients with EGFR-mutant NSCLC with inconsistent results⁴⁹⁻⁵² and serum AREG levels have been suggested as a predictor of resistance to Gefitinib in patients with advanced NSCLC⁵³. To evaluate the potential clinical translation of our findings, LLC^{AR+} tumor-bearing mice were intravenously treated with an AREG-targeting antibody (αAR). When combined with αAR, RT elicited a significant growth delay of flank LLC tumors (FIG. 5A) and the RT-induced increase in the size of lung metastases was abrogated (FIGS. 5B-5C). Additionally, EGFR-TKI treatment significantly abrogated the RT-induced metastatic growth when combined with αAR (FIGS. 5D-5F).

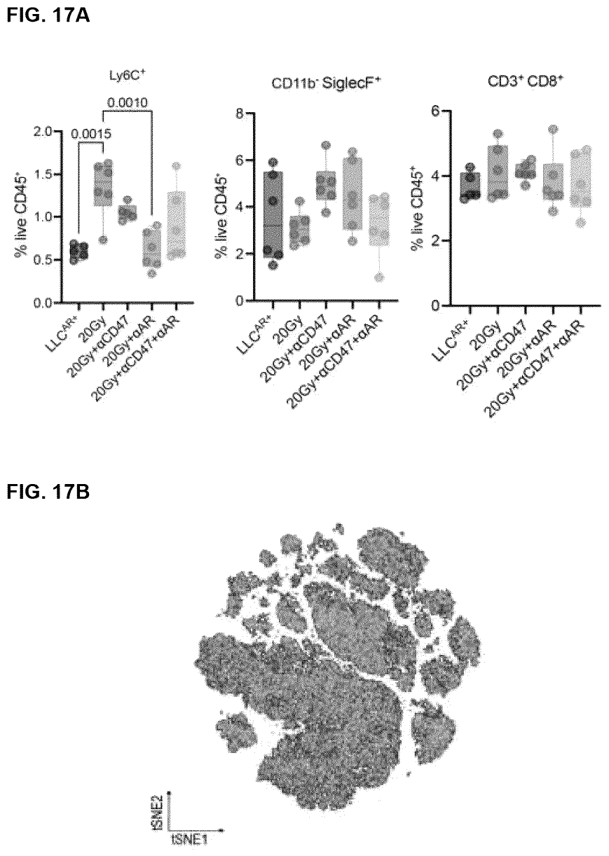

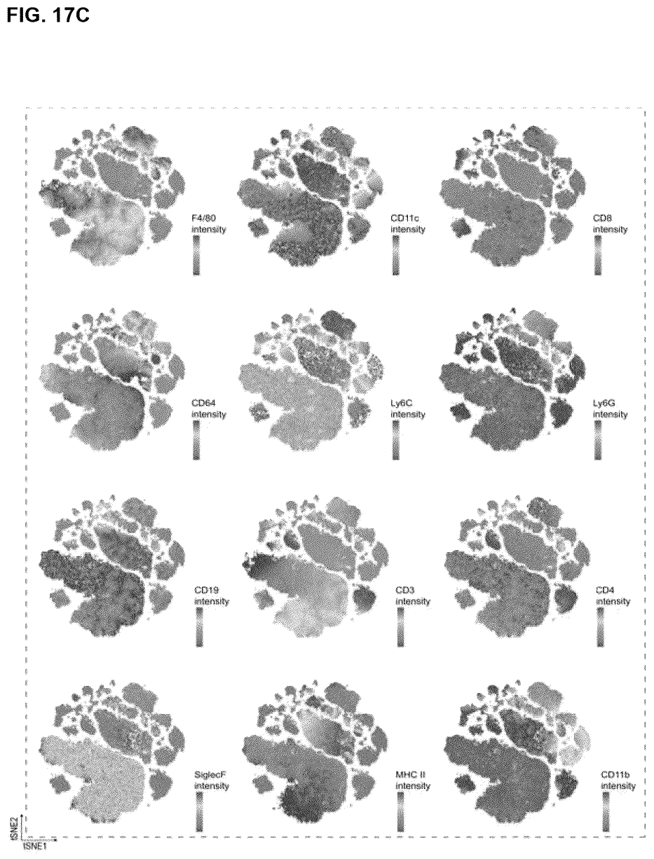

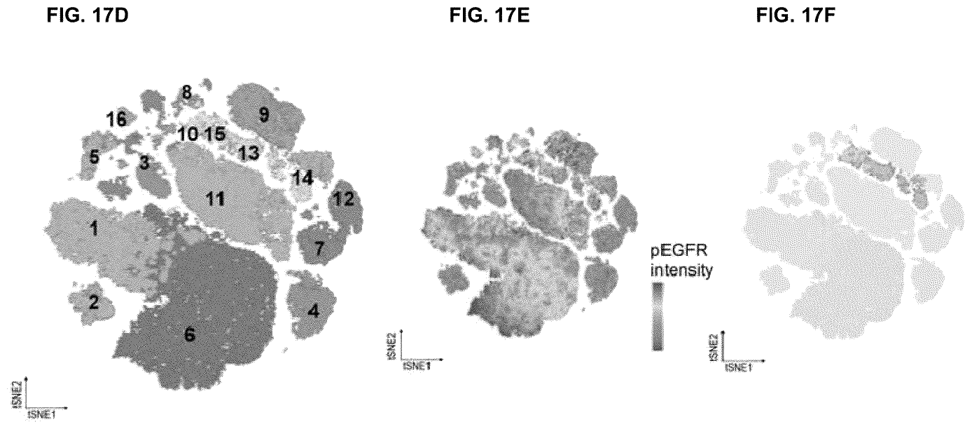

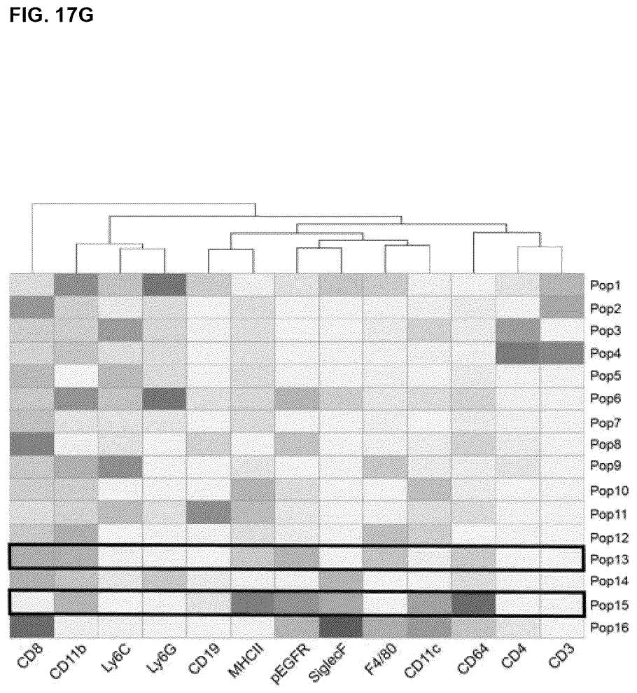

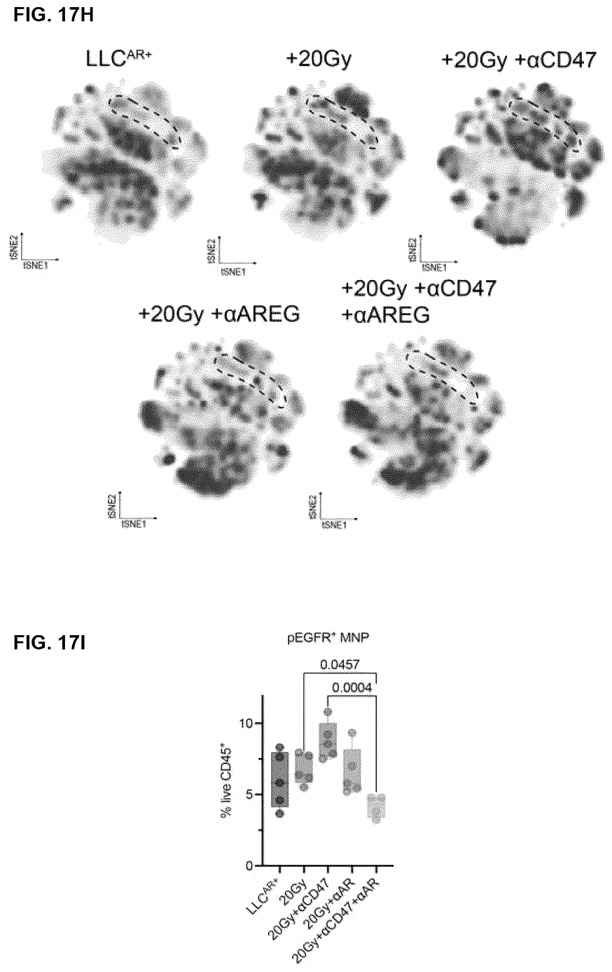

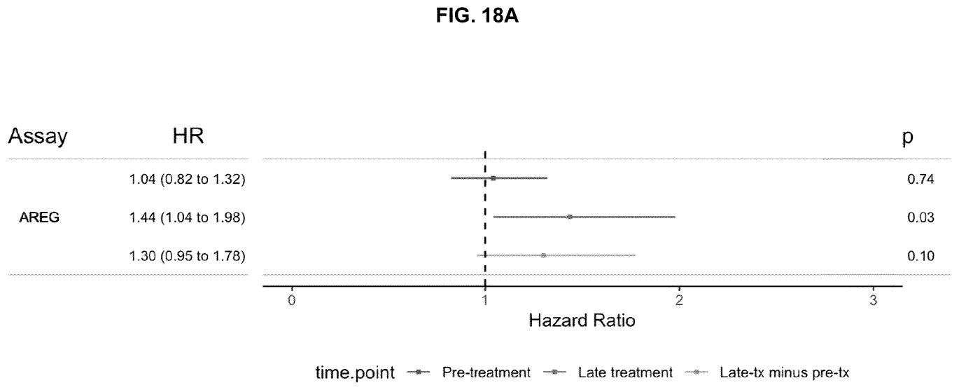

Since we observed AREG-induced, CD47-SIRPα mediated suppression of MNP phagocytosis, we tested whether αAR increased the efficacy of anti-CD47 (αCD47) immunotherapy in combination with IR. The combination treatment of αAR, αCD47 and RT significantly abrogated LLC^{AR+} flank tumor growth and reduced the size of metastasis (FIG. 5G-5H). Flow cytometry verified a significant increase of MNP phagocytosis after combination treatment (FIGS. 16B-16C) and a reduction of the RT-induced increase of pEGFR⁺ MNPs in murine lungs (FIGS. 17A-17I). Therefore, we concluded that AREG-blockade abrogates RT-induced metastatic proliferation and enhances RT efficacy alone as well as in combination with EGFR-TKI and αCD47 immunotherapy.

### DISCUSSION

Here we find that radiation-induced amphiregulin supports distant metastatic growth by reprogramming pEGFR⁺ myeloid cells and suppressing phagocytosis. Importantly, RT-induced AREG and pEGFR⁺ MNPs were detected in biopsies, serum, and PBMCs of cancer patients post-SBRT, findings recapitulated in murine lung metastasis models (FIGS. 2A-2F, FIGS. 10A-10C). These effects were overcome by AREG antibody blockade in mice, thereby inhibiting RT-induction of metastasis growth (FIGS. 17A-I).

Radiotherapy has first been described to increase tumor cell migration⁵⁴ and epithelial-to-mesenchymal transition⁵⁵ (EMT), hence facilitating metastasis in murine models^{56,57}. While most investigations have focused on the migratory and invasive potential of metastatic tumor cells⁵⁸, we report that local RT also increases the size of established distant metastases, a clinically-relevant mechanism that has garnered less attention. Our results suggest that this mechanism is most pronounced when local tumor control is inadequate, highlighting the need to identify and neutralize metastasis growth-promoting effects of RT to maximize therapeutic efficacy. These deleterious effects of RT may also obscure the anti-tumor benefits of other cancer treatments.

AREG was one of several radio-inducible genes associated with distant tumor progression in our clinical study. Therefore, we cannot rule out that other soluble factors may be involved in RT-mediated metastasis proliferation. Here we utilized relatively large radiation doses clinically relevant to SBRT. It is unclear whether these effects are observed at smaller doses (2 Gy) used over weeks, as in fractionated RT; however, the protracted treatment time might also provide more opportunities for identification of factors that potentially limit RT effectiveness.

Although immune cells other than MNPs may play a role in enhancing metastatic growth, our findings highlight a tumor-AREG-MNP-EGFR signaling loop that enables distant metastasis proliferation following IR. Tumor-derived AREG induced by distant RT alters monocyte differentiation in the lung, promoting monocyte arrest at an immature suppressive state and induces differentiation along a tumor-tolerogenic trajectory. AREG also modulates immune evasion by upregulating CD47 (FIGS. 15C-15D), reducing tumor cell phagocytosis and facilitating tumor cell survival, while AREG-blockade enhances anti-CD47 efficacy (FIG. 5G-H). CD47 blockade combined with RT has been shown to elicit a macrophage-mediated abscopal effect in small cell lung cancer (SCLC) models⁵⁹, which taken in concert with our findings suggests that the distant effects of RT on tumor growth are in part dependent on phagocytosis. EGFR signaling has been linked to the immunosuppressive and tumor-promoting roles of MNPs^{60,61}, and EGFR-targeted therapies have been shown to mitigate immunosuppression in the TME of inflammatory breast cancer⁶². Importantly, AREG has been proposed as a regulator of TAM accumulation in a breast cancer model⁶³.

Serum AREG concentration has been identified as a predictor of disease progression and PFS in metastatic colorectal cancer and NSCLC^{53,64,65}, in line with our results of improved PFS in patients with reduced serum AREG levels after SBRT+ICB. Notably, antibody-mediated targeting of AREG has also shown promising results in decreasing PD-L1-mediated immunosuppression in combination with Azetolizumab or Nivolumab⁶⁶. Our findings additionally suggest that AREG can modify the treatment response to EGFR TKI, a finding that has previously been shown to be relevant in NSCLC patients⁵³.

Taken together, we demonstrate for the first time that radiation-induced growth factors drive distant metastasis growth in SBRT-treated patients and murine models, which leads to shortened survival and adverse outcomes. We demonstrate that the mechanisms promoting distant metastasis growth post-IR are targetable, which suggests a clinical trial where adverse factors are assayed shortly after initiation of RT±immunotherapy and neutralized. These results indicate a paradigm shift for the use of RT in patients with locally advanced and metastatic tumors, leading to a new type of “personalized” RT.

### Example 6: Elevated Post-Treatment AREG Predicts Poor Survival in Metastatic NSCLC Patients Receiving RT and ICB

Radiotherapy (RT) induces amphiregulin (AREG) expression, which drives metastatic outgrowth through myeloid cell reprogramming in preclinical models. Whether AREG undermines the efficacy of RT combined with immune checkpoint blockade (ICB) in patients remains unknown. Here, we analyze two independent clinical cohorts and demonstrate that AREG expression associates with survival outcomes and immunosuppressive changes both within and beyond irradiated tumors. In the COSINR trial (NCT03223155) of metastatic non-small cell lung cancer (NSCLC) patients treated with stereotactic body radiation therapy (SBRT) and dual ICB (nivolumab plus ipilimumab), elevated post-RT serum AREG correlated with decreased overall survival, reduced tumor response, diminished T-cell receptor (TCR) repertoire diversity, contraction of effector T-cell populations, and expansion of classical monocytes. Notably, treating all measurable metastatic lesions with RT improved outcomes in high-AREG patients, suggesting that comprehensive local control may mitigate AREG-mediated immunosuppression. These findings establish AREG as a clinically relevant mediator of RT-induced immune dysfunction in the context of ICB and identify a biomarker-defined population that may benefit from AREG-EGFR pathway inhibition in both metastatic and definitive treatment settings.

Immunotherapy with immune checkpoint inhibitors has become a mainstay of treatment in many metastatic and locally advanced cancers. The durable responses observed in select metastatic cancer patients treated with immune checkpoint blockade (ICB) have generated considerable interest in combining ICB with radiotherapy (RT). However, while individual patients appear to have responded to RT plus ICB, trials across multiple tumor types have failed to demonstrate progression-free or overall survival benefits. Many of these trials investigated single-site stereotactic body radiotherapy (SBRT) using lower-dose regimens (often 8-9 Gy×3) or mandated that measurable lesions be left untreated to assess response. Although combined SBRT and ICB treatment appears safe, and a subgroup of NSCLC patients without PD-L1 expression may experience improved progression-free survival (PFS), these trials were otherwise uniformly negative for survival benefit among all enrolled patients.⁸⁷⁻⁹²

To extend the benefit of ICB patients with metastatic cancer receiving RT, further investigation is required to understand the mechanisms underlying the failure of these trials, including potential immunosuppressive effects of RT. RT induces expression of amphiregulin (AREG), an epidermal growth factor receptor (EGFR) ligand, which paradoxically promotes the growth of pre-existing metastases while decreasing the spread of new metastases.^{90, 93} AREG reprograms myeloid cells toward an immunosuppressive, pro-metastatic phenotype through EGFR signaling while upregulating CD47, an anti-phagocytosis signal in tumor cells. In murine models, AREG expression in the irradiated tumor microenvironment impaired systemic anti-tumor immunity and was associated with CD8+ T-cell exhaustion. Moreover, analysis of serum samples from a subset of patients in the COSINR trial revealed elevated AREG levels following RT, which correlated with phosphorylated EGFR (pEGFR) expression in circulating monocytes, validating the mechanistic link between AREG and myeloid cell activation in human patients.

Here, we report an analysis of the COSINR trial of metastatic NSCLC patients treated with SBRT plus nivolumab/ipilimumab. We demonstrate that AREG elevation predicts poor clinical outcomes, impairs both peripheral and tumor-infiltrating T-cell responses, and that comprehensive treatment of metastatic lesions can overcome AREG-mediated resistance in high-risk patients.

### Methods

### Patient Cohorts and Sample Collection

COSINR Trial (NCT03223155): Patients with de novo metastatic NSCLC without targetable driver mutations were enrolled in a Phase I/II trial combining SBRT (1-4 lesions) with nivolumab (3 mg/kg IV every 2 weeks) plus ipilimumab (1 mg/kg IV every 6 weeks). Patients were randomized to concurrent (ICB 1-14 days prior to SBRT) or sequential (ICB 1-7 days after SBRT) treatment arms. The concurrent arm proceeded to Phase II. Peripheral blood samples were collected at three timepoints: pre-treatment (before RT or ICB), early treatment (1-14 days from treatment start, post-SBRT in sequential arm), and late treatment (day 1 of cycle 3, at a median of 82 days from treatment start, after 2 cycles of ICB). All patients provided written informed consent.

### Serum Proteomics

Serum samples from COSINR patients underwent proteomic analysis using the Olink Proximity Extension Assay (Olink Proteomics, Uppsala, Sweden). Normalized protein expression values (NPX) were generated according to manufacturer protocols. AREG levels were extracted and analyzed across timepoints. Single-sample gene set enrichment analysis (ssGSEA) was performed using Hallmark and immune-related Reactome pathway gene sets to characterize proteomic signatures.

### Flow Cytometry

Peripheral blood mononuclear cells (PBMCs) were isolated from fresh blood samples and analyzed by multiparameter flow cytometry. Panels included markers for: (1) myeloid populations (monocyte subsets, MDSCs, polymorphonuclear cells); (2) lymphoid populations (CD20+ B cells, NK cells); and (3) T-cell subsets (CD4+, CD8+, regulatory T cells, effector memory populations, Th1/Th17 subsets). Gating strategies followed established protocols with appropriate fluorescence-minus-one controls.

### TCR Sequencing

Bulk TCR sequencing was performed on genomic DNA from PBMCs using the immunoSEQ Assay (Adaptive Biotechnologies, Seattle, WA) according to manufacturer protocols. TCR β-chain CDR3 regions were amplified and sequenced. Metrics including clonality (1—normalized Shannon entropy), clonotype richness (number of unique productive TCR sequences), and clone tracking across timepoints were computed. Repertoire dynamics were assessed by quantifying new clonotypes emerging post-treatment and pruning of pre-existing clones.

### TCR Sequencing of Tumor Samples

TCR sequencing was performed on DNA extracted from tumor tissue using immunoSEQ (Adaptive Biotechnologies) for peripheral blood in the trial. Metrics including productive rearrangement frequency and clonotype richness were calculated.

### Statistical Analysis

Overall survival was calculated from treatment start to death or last follow-up. Progression-free survival was calculated from treatment start to progression or death. Optimal cutpoints for continuous variables (AREG, ΔAREG) were determined using maximally selected rank statistics (maxstat package in R). Survival curves were compared using log-rank tests. Hazard ratios were calculated using Cox proportional hazards models. Comparisons between groups for continuous variables used Mann-Whitney U tests appropriate. Correlations were assessed using Spearman's rank correlation. Multiple testing correction was performed using Benjamini-Hochberg false discovery rate where appropriate. Statistical significance was defined as p<0.05. Analyses were performed using R version 4.x and GraphPad Prism 9.

### Results

The COSINR trial (Phase I/II) enrolled patients with de novo metastatic NSCLC without targetable driver mutations, randomizing them to receive nivolumab (3 mg/kg every 2 weeks) plus ipilimumab (1 mg/kg every 6 weeks) with SBRT (1-4 lesions) administered either concurrently (ICB 1-14 days prior to SBRT) or sequentially (ICB initiated 1-7 days after SBRT completion). To facilitate comparison with the Pembro-SBRT trial, which delivered multi-site SBRT followed by pembrolizumab, we focused our primary analysis on the sequential treatment arm (n=19 total, n=16 with post-treatment proteomic data) to maintain consistency with the published paradigm.

We measured serum AREG levels using the Olink proximity extension assay at two timepoints: pre-treatment (before any RT or ICB) and on-treatment (day 1 of cycle 3, approximately 70-80 days from treatment start, after 2 cycles of ICB). Importantly, these samples represent an independent cohort from the patients analyzed by ELISA,⁹³ providing external validation using an orthogonal proteomic platform.

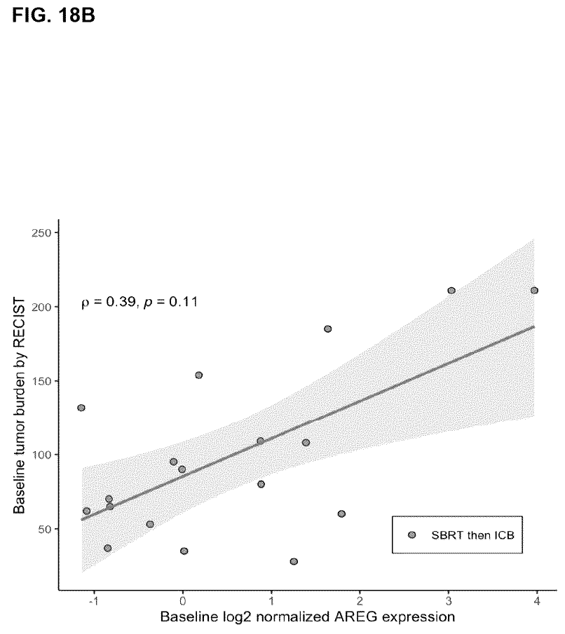

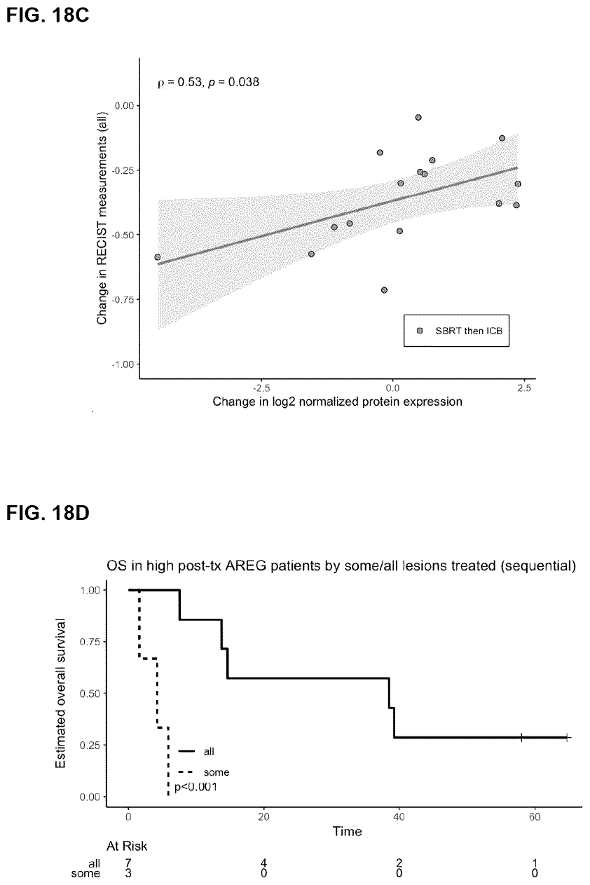

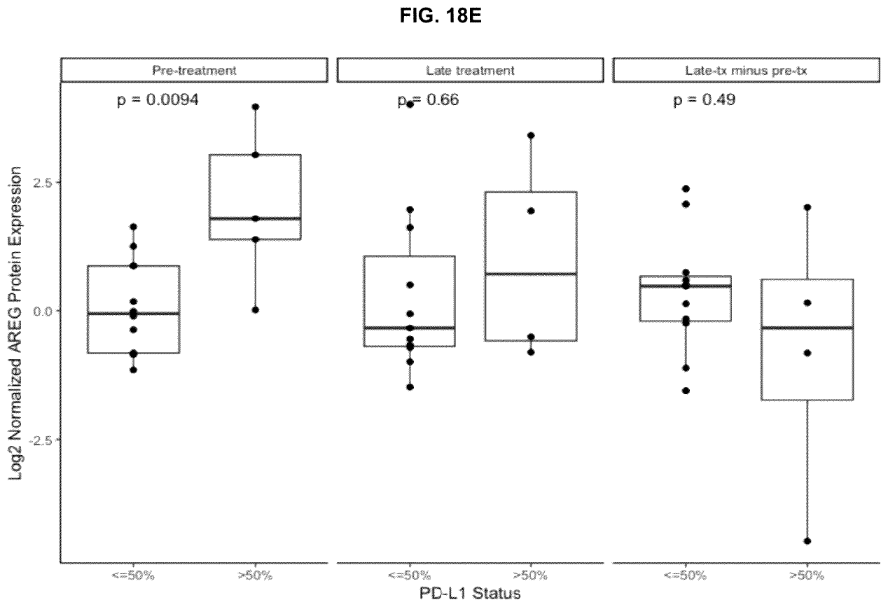

Consistent with previous findings, elevated late-treatment AREG (approximately 70-80 days post-treatment initiation) was significantly associated with inferior overall survival. Landmarked Cox regression identified that increasing post-treatment AREG was associated with worse overall survival (HR 1.82, 95% CI 1.16-2.87), as was an increasing difference between post-treatment and pre-treatment AREG expression (HR 1.68, 95% CI 1.05-2.69) (FIG. 18A).

Unlike Pembro-SBRT, COSINR did not mandate that one or more sites of metastatic disease be left untreated to assess response. While pre-treatment tumor volume was not associated with pre-treatment AREG expression (FIG. 18B), post-treatment AREG levels correlated inversely with tumor response. Patients with high late-treatment AREG showed smaller reductions in RECIST-measured target lesion diameters (FIG. 18C), suggesting AREG production by residual tumor with incomplete response to treatment.

### Comprehensive RT of Metastatic Lesions Improves Outcomes in High-AREG Patients

A critical clinical question is whether treatment strategy modifications can overcome AREG-mediated resistance. To address this, we examined overall survival stratified by whether patients had all measurable sites of disease treated. Among patients with high post-treatment AREG, treating all RECIST-measurable metastatic sites with RT was associated with significantly improved OS (FIG. 18D). These findings support a model wherein AREG-driven growth of metastases contributes to poor outcomes, which can be mitigated by ablating these sites with radiotherapy.

### Tumor Histology and PD-L1 Status Influence AREG Expression Dynamics

Analysis of AREG expression patterns revealed associations with baseline tumor PD-L1 status. Notably, patients with PD-L1-high tumors (tumor proportion score ≥50%) exhibited elevated pre-treatment serum AREG levels compared to PD-L1-low patients (FIG. 18E), suggesting a potential link between baseline immune contexture and AREG expression.

High Post-Treatment AREG Associates with Immunosuppressive Peripheral Blood Proteomic Signatures

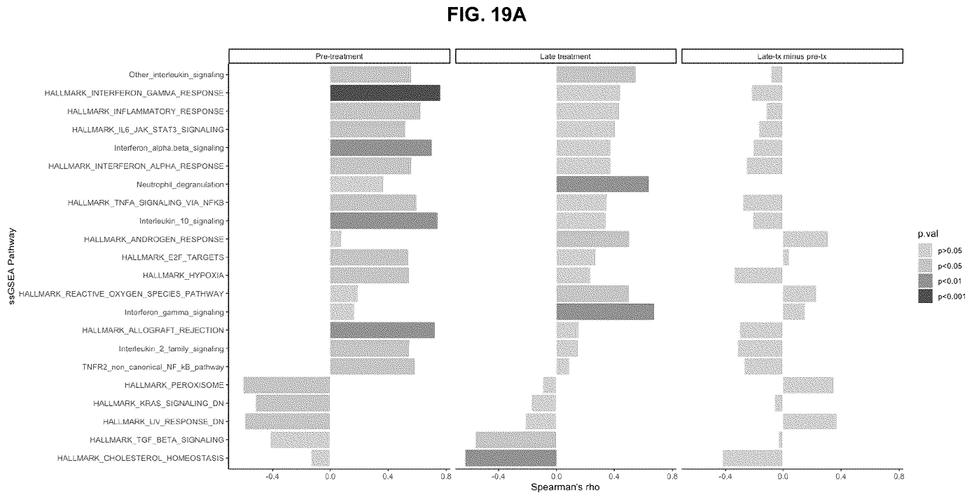

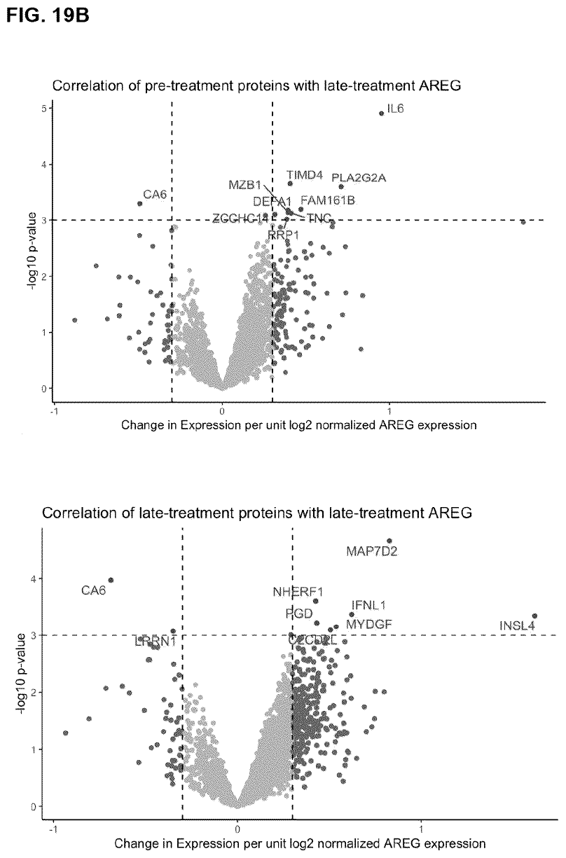

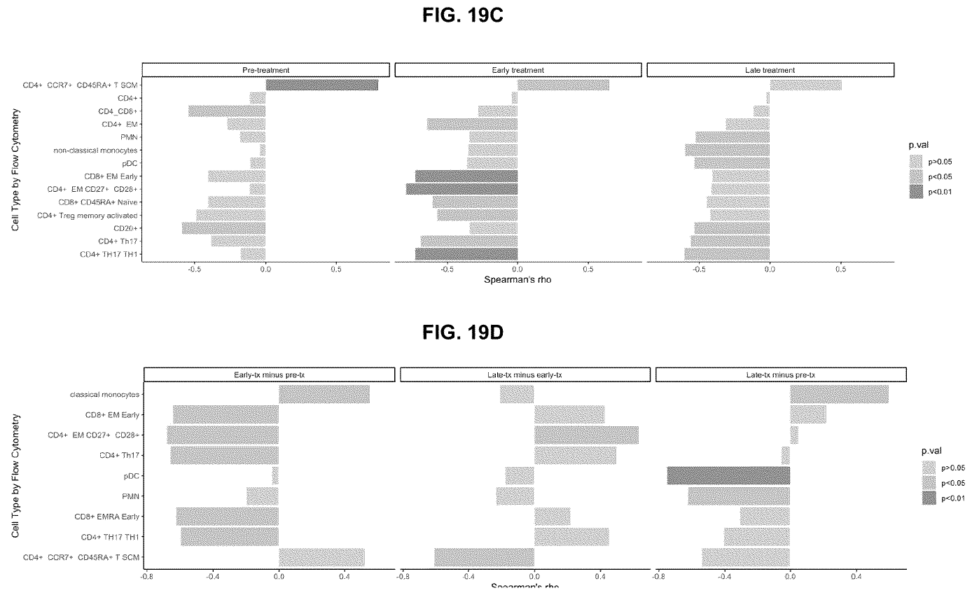

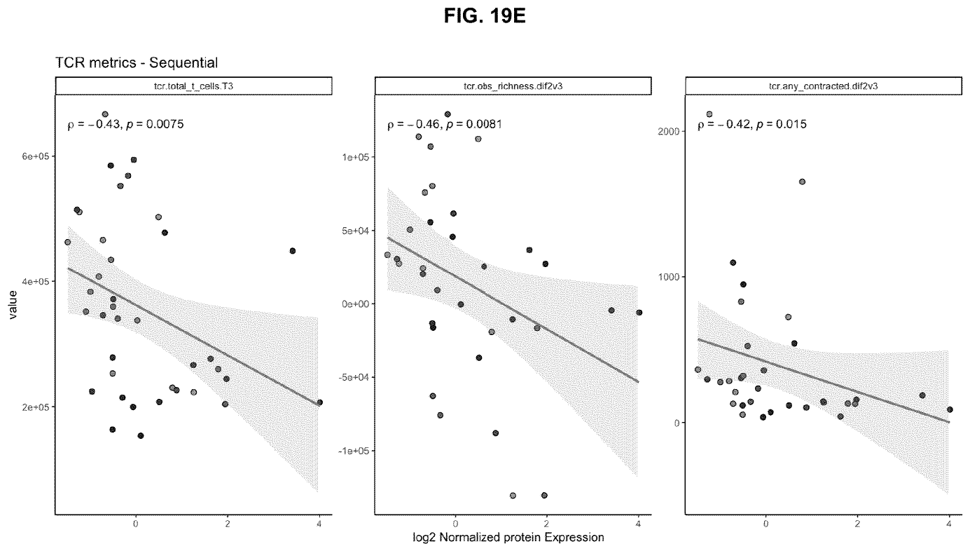

To investigate immune parameter changes in the context of elevated AREG, we performed single-sample gene set enrichment analysis (ssGSEA) on peripheral blood proteomic profiles (Olink) using the Hallmark gene sets and a subset of immune-specific Reactome pathways curated by ImmPort. Pathway analysis revealed that post-treatment AREG positively correlated with baseline interferon type I and type II signaling, IL-6 signaling, and NFκB pathway activation (FIG. 19A). Conversely, FLT3 signaling, previously associated with enhanced immune infiltration in NSCLC, correlated with lower post-treatment AREG levels. Post-treatment proteomic analysis in high-AREG patients revealed striking enrichment of hypoxia-associated pathways, including glycolysis, the unfolded protein response, and Hallmark hypoxia signatures (FIG. 19A). Correlation of pre- and post-treatment proteins with post-treatment AREG was analyzed (FIG. 19B). Pre-treatment IL6 and TIMD4 and post-treatment IFNL1 and MAP7D2 were positively correlated with post-treatment AREG. Carbonic anhydrase 6 (CA6) was negatively correlated with post-treatment AREG.

### Flow Cytometry Reveals AREG-Associated Shifts in Peripheral Immune Cell Populations

We next examined peripheral blood immune cell populations by flow cytometry at all three timepoints (pre-treatment, early treatment, and late treatment) in sequential arm patients (n=16 with T-cell subset data). Increasing post-treatment AREG positively correlated with an increase in classical monocyte markers from pre- to post-treatment (FIG. 19C), consistent with our previous findings of AREG-mediated myeloid reprogramming and elevated pEGFR in monocytes. Conversely, lower post-treatment AREG levels were associated with early mobilization of CD4+ and CD8+ effector memory T cells and enrichment of Th17 and Th1 cell populations. Additionally, both plasmacytoid and myeloid dendritic cells measured at late treatment correlated with reduced AREG levels (FIG. 19C). These findings demonstrate that elevated AREG associates with a shift in peripheral immune composition characterized by expansion of immunosuppressive myeloid populations and depletion of effector T-cell subsets critical for anti-tumor immunity.

High AREG Levels Correlate with Impaired T-Cell Responses and Reduced TCR Diversity

Given the established role of T cells as primary effectors of ICB, we performed bulk TCR sequencing (immunoSEQ, Adaptive Biotechnologies) on peripheral blood samples from 16 patients across all timepoints. Elevated late-treatment AREG significantly correlated with lower post-treatment T-cell counts and reduced TCR clonotype richness compared to low-AREG patients. Importantly, high-AREG patients were more likely to experience progressive decline in TCR richness from early to late treatment timepoints (FIG. 19D), suggesting ongoing immune repertoire collapse despite ICB therapy.

Analysis of TCR dynamics revealed that high-AREG patients exhibited less repertoire “dynamism,” characterized by diminished contraction in the quantity of TCR clonotypes between early and late timepoints (FIG. 19D). This frozen repertoire state is consistent with T-cell exhaustion and impaired immune surveillance, recapitulating previous preclinical findings of AREG-induced T-cell dysfunction in murine lung metastases.⁹³

These data establish that elevated AREG not only correlates with poor clinical outcomes but mechanistically impairs the T-cell response to combined RT and ICB, consistent with the hypothesis that radiotherapy-induced amphiregulin may be immunosuppressive and interfere with RT-ICB synergy.

Several key findings emerge from our analysis. First, we demonstrate that elevated serum AREG following RT and ICB predicts poor survival in metastatic NSCLC patients, validating previous ELISA-based measurements using an independent cohort and orthogonal proteomic platform (Olink).⁹³ The design of the COSINR trial allowed for treatment of all measurable disease, precluding evaluation of untreated metastatic site growth as in a previous report⁹³ (only n=4 in this cohort had fewer than all RECIST sites treated). However, elevated post-treatment AREG was associated with poorer response to treatment in both treated and untreated lesions, suggesting that treatment-induced AREG facilitates persistence of metastatic disease through treatment- or alternatively, that untreated disease acts as a reservoir for AREG production. The consistent association with survival and metastatic lesion response reinforces the prognostic potential of AREG as a biomarker in patients with metastatic cancer.

Elevated AREG correlates with expansion of immunosuppressive classical monocytes, depletion of CD8+ effector T-cell populations, reduced TCR diversity, and diminished TCR dynamism. These findings extend preclinical observations of AREG-driven myeloid reprogramming and T-cell exhaustion⁹³ to the clinical setting, establishing the translational relevance of these mechanisms.

Our finding that comprehensive RT coverage of metastatic lesions improves outcomes specifically in high-AREG metastatic NSCLC patients has important clinical implications. This observation suggests that AREG-driven growth of untreated metastases contributes substantially to disease progression and that more extensive local therapy can overcome this resistance mechanism. Our findings suggest that AREG acts as an immunosuppressive counterbalance to previously identified RT-induced immunogenic changes, which could explain the overall lack of benefit in these studies. These data support consideration of total ablation of all metastatic lesions when feasible, particularly in patients identified as AREG-high.

Considered alongside previous work,⁹³ our results support a model whereby AREG—whether induced by RT or constitutively expressed by tumor cells—activates EGFR signaling in myeloid cells, promoting their differentiation toward immunosuppressive phenotypes. These reprogrammed myeloid cells then impair T-cell priming, trafficking, and effector function through multiple mechanisms, including checkpoint ligand expression, metabolic competition, and secretion of immunosuppressive factors. The enrichment of hypoxia signatures in high-AREG patients suggests additional metabolic constraints on T-cell function.

The observation that PD-L1-high patients exhibit elevated baseline AREG may reflect pre-existing inflammatory states that prime for AREG induction. Whether PD-L1 and AREG represent sequential or parallel resistance mechanisms warrants further investigation, as combined targeting of both pathways may be required in some patients.

Several limitations of our study should be acknowledged. The COSINR trial sample size was small, and validation in larger prospective cohorts of SBRT combined with immunotherapy in patients with metastatic disease is needed. Additionally, while we demonstrate associations between AREG and immune dysfunction, direct causation in human samples cannot be established, though our prior preclinical studies provide strong mechanistic evidence.⁹³ Our results do not directly address the effects of AREG on tumor cells themselves; AREG was shown to activate CD47,⁹³ and recent work has demonstrated that AREG produced by suppressive myeloid cells alters epithelial-mesenchymal transition.⁹⁴

Future work should investigate: (1) prospective validation of AREG as a predictive biomarker in ongoing RT-ICB trials; (2) development of AREG-EGFR pathway inhibitors or neutralizing antibodies as combination strategies; (3) investigation of alternative treatment sequencing or patient selection strategies based on AREG status; and (4) deeper mechanistic studies defining AREG-responsive myeloid and T-cell subpopulations using single-cell technologies.

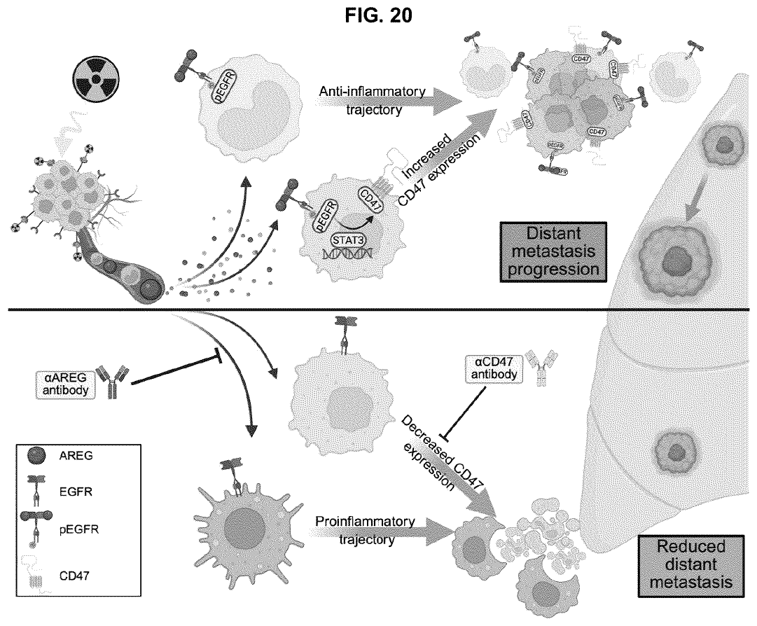

In conclusion, these findings establish AREG as a critical mediator of RT-induced immune suppression and a robust biomarker identifying patients with impaired response to ICB (FIG. 20). These findings provide a mechanistic framework for understanding RT-ICB combination therapy failures and identify specific patient populations who may benefit from AREG pathway inhibition or modified treatment strategies. AREG measurements may enable rational patient selection and inform treatment intensification decisions in metastatic disease.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification, and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth herein.

### REFERENCES

ADDIN ZOTERO_BIBL {“uncited”: [ ], “omitted”: [ ], “custom”: [ ]} CSL_BIBLIOGRAPHY 1.

Orth, M. et al. Current concepts in clinical radiation oncology. Radiation and Environmental Biophysics 53, 1-29 (2014).
2. Lomax, M. E., Folkes, L. K. & O'Neill, P. Biological Consequences of Radiation-induced DNA Damage: Relevance to Radiotherapy. Clinical Oncology 25, 578-585 (2013).
3. Lynch, C., Pitroda, S. P. & Weichselbaum, R. R. Radiotherapy, immunity, and immune checkpoint inhibitors. The Lancet Oncology 25, e352-e362 (2024).
4. Deng, L. et al. STING-dependent cytosolic DNA sensing promotes radiation-induced type I interferon-dependent antitumor immunity in immunogenic tumors. Immunity 41, 843-852 (2014).
5. Liang, H. et al. Radiation-Induced Equilibrium Is a Balance between Tumor Cell Proliferation and T Cell-Mediated Killing. The Journal of Immunology 190, 5874-5881 (2013).
6 Liang, H. et al. Host STING-dependent MDSC mobilization drives extrinsic radiation resistance. Nature Communications 8, 1-10 (2017).
7. Jiménez-Cortegana, C., Galassi, C., Klapp, V., Gabrilovich, D. I. & Galluzzi, L. Myeloid-Derived Suppressor Cells and Radiotherapy. Cancer Immunology Research 10, 545-557 (2022).
8. Pitroda, S. P., Chmura, S. J. & Weichselbaum, R. R. Integration of radiotherapy and immunotherapy for treatment of oligometastases. The Lancet Oncology 20, e434-e442 (2019).
9. Katipally, R. R., Pitroda, S. P., Juloori, A., Chmura, S. J. & Weichselbaum, R. R. The oligometastatic spectrum in the era of improved detection and modern systemic therapy. Nature Reviews Clinical Oncology 19, 585-599 (2022).
10. Weichselbaum, R. R., Liang, H., Deng, L. & Fu, Y. X. Radiotherapy and immunotherapy: A beneficial liaison? Nature Reviews Clinical Oncology 14, 365-379 (2017).
11. Pointer, K. B., Pitroda, S. P. & Weichselbaum, R. R. Radiotherapy and immunotherapy: open questions and future strategies. Trends in Cancer 8, 9-20 (2022).
12. Wang, L. et al. RNA m6A methylation and MDSCs: Roles and therapeutic implications for radiotherapy. Med 4, 863-874 (2023).
13. Shoyab, M., Plowman, G., McDonald, V., Garrett Bradley, J. & Todaro, G. J. Structure and Function of Human Amphiregulin: A Member of the Epidermal Growth Factor Family. Science 243, 1074-76 (1989).
14. Shoyab, M., McDonald, V. L., Bradley, J. G. & Todaro, G. J. Amphiregulin: A bifunctional growth-modulating glycoprotein produced by the phorbol 12-myristate 13-acetate-treated human breast adenocarcinoma cell line MCF-7. Proceedings of the National Academy of Sciences of the United States of America 85, 6528-6532 (1988).
15. Zaiss, D. M. W. et al. Amphiregulin Enhances Regulatory T Cell-Suppressive Function via the Epidermal Growth Factor Receptor. Immunity 38, 275-284 (2013).
16. Zaiss, D. M. W., Gause, W. C., Osborne, L. C. & Artis, D. Emerging functions of amphiregulin in orchestrating immunity, inflammation, and tissue repair. Immunity 42, 216-226 (2015).
17. Minutti, C. M. et al. Epidermal Growth Factor Receptor Expression Licenses Type-2 Helper T Cells to Function in a T Cell Receptor-Independent Fashion. Immunity 47, 710-722.e6 (2017).
18. Busser, B., Sancey, L., Brambilla, E., Coll, J. L. & Hurbin, A. The multiple roles of amphiregulin in human cancer. Biochimica et Biophysica Acta—Reviews on Cancer 1816, 119-131 (2011).
19. Berasain, C. & Avila, M. A. Amphiregulin. Seminars in Cell and Developmental Biology 28, 31-41 (2014).
20. Shao, J. & Sheng, H. Amphiregulin promotes intestinal epithelial regeneration: Roles of intestinal subepithelial myofibroblasts. Endocrinology 151, 3728-3737 (2010).
21. Luke, J. J. et al. Safety and clinical activity of pembrolizumab and multisite stereotactic body radiotherapy in patients with advanced solid tumors. Journal of Clinical Oncology 36, 1611-1618 (2018).
22. Luke, J. J. et al. Improved survival associated with local tumor response following multisite radiotherapy and pembrolizumab: Secondary analysis of a phase i trial. Clinical Cancer Research 26, 6437-6444 (2020).
23. Mulder, K. et al. Cross-tissue single-cell landscape of human monocytes and macrophages in health and disease. Immunity 54, 1883-1900.e5 (2021).
24. Georgiades, P. et al. VavCre transgenic mice: a tool for mutagenesis in hematopoietic and endothelial lineages. Genesis (New York, N.Y.: 2000) 34, 251-256 (2002).
25. Johnstone, C. N. et al. Functional and molecular characterisation of EO771.LMB tumours, a new C57BL/6-mouse-derived model of spontaneously metastatic mammary cancer. DMM Disease Models and Mechanisms 8, 237-251 (2015).
26. McElrath, C. et al. Critical role of interferons in gastrointestinal injury repair. Nat Commun 12, 2624 (2021).
27. Gilmore, J. L. et al. Amphiregulin-EGFR signaling regulates PTHrP gene expression in breast cancer cells. Breast Cancer Research and Treatment 110, 493-505 (2008).
28. Miyawaki, M. et al. Enhanced phosphorylation of the epidermal growth factor receptor at the site of tyrosine 992 in esophageal carcinomas. APMIS 116, 1097-1106 (2008).
29. Bestvina, C. M. et al. A Phase 1 Trial of Concurrent or Sequential Ipilimumab, Nivolumab, and Stereotactic Body Radiotherapy in Patients With Stage IV NSCLC Study. Journal of Thoracic Oncology 17, 130-140 (2022).
30. Van Der Maaten, L. & Hinton, G. Visualizing Data using t-SNE. Journal of Machine Learning Research 9, 2579-2605 (2008).
31. Swirski, F. K., Hilgendorf, I. & Robbins, C. S. From proliferation to proliferation: Monocyte lineage comes full circle. Seminars in Immunopathology 36, 137-148 (2014).
32. Noy, R. & Pollard, J. W. Tumor-Associated Macrophages: From Mechanisms to Therapy. Immunity 41, 49-61 (2014).
33. Wang, T. et al. Influenza-trained mucosal-resident alveolar macrophages confer long-term antitumor immunity in the lungs. Nature Immunology 1-16 (2023) doi: 10.1038/s41590-023-01428-x.
34. Wang, L. et al. YTHDF2 inhibition potentiates radiotherapy antitumor efficacy. Cancer Cell 1-15 (2023) doi: 10.1016/j.ccell.2023.04.019.
35. Miller, B. C. et al. Subsets of exhausted CD8+ T cells differentially mediate tumor control and respond to checkpoint blockade. Nature Immunology 20, 326-336 (2019).
36. Mack, M. et al. Expression and characterization of the chemokine receptors CCR2 and CCR5 in mice. J Immunol 166, 4697-4704 (2001).
37. Lecoultre, M., Dutoit, V. & Walker, P. R. Phagocytic function of tumor-associated macrophages as a key determinant of tumor progression control: A review. Journal for Immuno Therapy of Cancer 8, e001408 (2020).
38. Wang, Y. et al. Intravenous Delivery of siRNA Targeting CD47 Effectively Inhibits Melanoma Tumor Growth and Lung Metastasis. Molecular Therapy 21, 1919-1929 (2013).
39. Loyher, P. L. et al. Macrophages of distinct origins contribute to tumor development in the lung. Journal of Experimental Medicine 215, 1-18 (2018).
40. Gonzalez, M. A. et al. Phagocytosis increases an oxidative metabolic and immune suppressive signature in tumor macrophages. Journal of Experimental Medicine 220, (2023).
41. Platen, C., Dreschers, S., Reiss, L. K., Wappler, J. & Orlikowsky, T. W. Amphiregulin Regulates Phagocytosis-Induced Cell Death in Monocytes via EGFR and Matrix Metalloproteinases. Mediators of Inflammation 2018, (2018).
42. Hsu, Y.-L. et al. Lung Tumor-Associated Dendritic Cell-Derived Amphiregulin Increased Cancer Progression. The Journal of Immunology 187, 1733-1744 (2011).
43. Ebott, J. et al. Enhanced amphiregulin exposure promotes modulation of the high grade serous ovarian cancer tumor immune microenvironment. Front. Pharmacol. 15, (2024).
44. Lu, J. et al. Reprogramming of TAMs via the STAT3/CD47-SIRPα axis promotes acquired resistance to EGFR-TKIs in lung cancer. Cancer Letters 564, 216205 (2023).
45. Schust, J., Sperl, B., Hollis, A., Mayer, T. U. & Berg, T. Stattic: a small-molecule inhibitor of STAT3 activation and dimerization. Chem Biol 13, 1235-1242 (2006).
46. Jaiswal, S. et al. CD47 Is Upregulated on Circulating Hematopoietic Stem Cells and Leukemia Cells to Avoid Phagocytosis. Cell 138, 271-285 (2009).
47. Weiskopf, K. et al. Engineered SIRPα Variants as Immunotherapeutic Adjuvants to Anticancer Antibodies. Science 341, 88-91 (2013).
48. Gordon, S. Phagocytosis: An Immunobiologic Process. Immunity 44, 463-475 (2016).
49. Ready, N. et al. Chemoradiotherapy and gefitinib in stage III non-small cell lung cancer with epidermal growth factor receptor and KRAS mutation analysis: Cancer and Leukemia Group B (CALEB) 30106, a CALGB-Stratified Phase II Trial. Journal of Thoracic Oncology 5, 1382-1390 (2010).
50. Kelly, K. et al. Phase III trial of maintenance gefitinib or placebo after concurrent chemoradiotherapy and docetaxel consolidation in inoperable stage III non-small-cell lung cancer: SWOG S0023. Journal of Clinical Oncology 26, 2450-2456 (2008).
51. Hotta, K. et al. Gefitinib induction followed by chemoradiotherapy in EGFR-mutant, locally advanced non-small-cell lung cancer: LOGIK0902/OLCSG0905 phase II study. ESMO Open 6, 100191 (2021).
52. Wrona, A., Dziadziuszko, R. & Jassem, J. Combining radiotherapy with targeted therapies in non-small cell lung cancer: Focus on anti-EGFR, anti-ALK and anti-angiogenic agents. Translational Lung Cancer Research 10, 2032-2047 (2021).
53. Ishikawa, N. et al. Increases of amphiregulin and transforming growth factor-α in serum as predictors of poor response to gefitinib among patients with advanced non-small cell lung cancers. Cancer Research 65, 9176-9184 (2005).
54. Vilalta, M., Rafat, M. & Graves, E. E. Effects of radiation on metastasis and tumor cell migration. Cellular and Molecular Life Sciences 73, 2999-3007 (2016).
55. Gomez-Casal, R. et al. Non-small cell lung cancer cells survived ionizing radiation treatment display cancer stem cell and epithelial-mesenchymal transition phenotypes. Molecular Cancer 12, (2013).
56. Kaplan, H. S. & Murphy, E. D. The effect of local roentgen irradiation on the biological behavior of a transplantable mouse carcinoma. i. increased frequency of pulmonary metastasis. Journal of the National Cancer Institute 9, 407-413 (1949).
57. Blyth, B. J., Cole, A. J., MacManus, M. P. & Martin, O. A. Radiation therapy-induced metastasis: radiobiology and clinical implications. Clinical and Experimental Metastasis 35, 223-236 (2018).
58. Sundahl, N., Duprez, F., Ost, P., De Neve, W. & Mareel, M. Effects of radiation on the metastatic process. Molecular Medicine 24, 1-20 (2018).
59. Nishiga, Y. et al. Radiotherapy in combination with CD47 blockade elicits a macrophage-mediated abscopal effect. Nature Cancer 1-16 (2022) doi: 10.1038/s43018-022-00456-0.
60. Lanaya, H. et al. EGFR has a tumour-promoting role in liver macrophages during hepatocellular carcinoma formation. Nature Cell Biology 16, 972-981 (2014).
61. Zhang, W. et al. Polarization of macrophages in the tumor microenvironment is influenced by EGFR signaling within colon cancer cells. Oncotarget 7, 75366-75378 (2016).
62. Wang, X. et al. EGFR is a master switch between immunosuppressive and immunoactive tumor microenvironment in inflammatory breast cancer. Science Advances 8, (2022).
63. Nickerson, N. K., Mill, C. P., Wu, H. J., Riese, D. J. & Foley, J. Autocrine-Derived Epidermal Growth Factor Receptor Ligands Contribute to Recruitment of Tumor-Associated Macrophage and Growth of Basal Breast Cancer Cells In Vivo. Oncology research 20, 303 (2013).
64. Chayangsu, C., Khunsri, S., Sriuranpong, V. & Tanasanvimon, S. The correlations between serum amphiregulin and other clinicopathological factors in colorectal cancer. Journal of Gastrointestinal Oncology 8, 980-984 (2017).
65. Kim, S. A. et al. Amphiregulin can predict treatment resistance to palliative first-line cetuximab plus FOLFIRI chemotherapy in patients with RAS wild-type metastatic colorectal cancer. Scientific Reports 11, 1-11 (2021).
66. Xu, Q. et al. Targeting amphiregulin (AREG) derived from senescent stromal cells diminishes cancer resistance and averts programmed cell death 1 ligand (PD-L1)-mediated immunosuppression. Aging Cell 18, (2019).
67. Jaffe, C. C. Measures of response: RECIST, WHO, and new alternatives. Journal of Clinical Oncology 24, 3245-3251 (2006).
68. Lee, T.-C. & Threadgill, D. W. Generation and validation of mice carrying a conditional allele of the epidermal growth factor receptor. Genesis 47, 85-92 (2009).
69. Stegen, B. et al. Contrast-enhanced, conebeam CT-based, fractionated radiotherapy and follow-up monitoring of orthotopic mouse glioblastoma: A proof-of-concept study. Radiation Oncology 15, 1-10 (2020).
70. Stringer, C., Wang, T., Michaelos, M. & Pachitariu, M. Cellpose: a generalist algorithm for cellular segmentation. Nat Methods 18, 100-106 (2021).
71. Pachitariu, M. & Stringer, C. Cellpose 2.0: how to train your own model. Nat Methods 19, 1634-1641 (2022).
72. Youden, W. J. Index for rating diagnostic tests. Cancer 3, 32-35 (1950).
73. Otsu, N. A Threshold Selection Method from Gray-Level Histograms. IEEE Transactions on Systems, Man, and Cybernetics 9, 62-66 (1979).
74. Levine, J. H. et al. Data-Driven Phenotypic Dissection of AML Reveals Progenitor-like Cells that Correlate with Prognosis. Cell 162, 184-197 (2015).
75. Van Gassen, S. et al. FlowSOM: Using self-organizing maps for visualization and interpretation of cytometry data. Cytometry Part A 87, 636-645 (2015).
76. Li, B. et al. Cumulus provides cloud-based data analysis for large-scale single-cell and single-nucleus RNA-seq. Nature Methods 17, 793-798 (2020).
77. Wolf, F. A., Angerer, P. & Theis, F. J. SCANPY: large-scale single-cell gene expression data analysis. Genome Biology 19, (2018).
78. Satija, R., Farrell, J. A., Gennert, D., Schier, A. F. & Regev, A. Spatial reconstruction of single-cell gene expression data. Nature Biotechnology 33, 495-502 (2015).
79. Korsunsky, I. et al. Fast, sensitive and accurate integration of single-cell data with Harmony. Nature Methods 16, 1289-1296 (2019).
80. Street, K. et al. Slingshot: Cell lineage and pseudotime inference for single-cell transcriptomics. BMC Genomics 19, 1-16 (2018).
81. La Manno, G. et al. RNA velocity of single cells. Nature 2018 560:7719 560, 494-498 (2018).
82. Bergen, V., Lange, M., Peidli, S., Wolf, F. A. & Theis, F. J. Generalizing RNA velocity to transient cell states through dynamical modeling. Nat Biotechnol 38, 1408-1414 (2020).
83. Gillespie, M. et al. The reactome pathway knowledgebase 2022. Nucleic Acids Research 50, D687-D692 (2022).
84. Kanehisa, M. & Goto, S. KEGG: Kyoto Encyclopedia of Genes and Genomes. Nucleic Acids Research 28, 27 (2000).
85. pegasus/signature_score.pdf at master·lilab-bcb/pegasus·GitHub.
86 Goldman, M. J. et al. Visualizing and interpreting cancer genomics data via the Xena platform. Nature Biotechnology 38, 675-678 (2020).
87. McBride, S. et al. Randomized Phase II Trial of Nivolumab With Stereotactic Body Radiotherapy Versus Nivolumab Alone in Metastatic Head and Neck Squamous Cell Carcinoma. J. Clin. Oncol. 39, 30-37 (2021).
88. Kim, S. et al. Combined nivolumab and ipilimumab with or without stereotactic body radiation therapy for advanced Merkel cell carcinoma: a randomised, open label, phase 2 trial. The Lancet 400, 1008-1019 (2022).
89. Theelen, W. S. M. E. et al. Effect of Pembrolizumab After Stereotactic Body Radiotherapy vs Pembrolizumab Alone on Tumor Response in Patients With Advanced Non-Small Cell Lung Cancer. JAMA Oncol. 5, 1276-1282 (2019).
90. Pakkala, S. et al. Durvalumab and tremelimumab with or without stereotactic body radiation therapy in relapsed small cell lung cancer: a randomized phase II study. J. Immunother. Cancer 8, e001302 (2020).
91. Chen, I. M. et al. Phase 2 study of ipilimumab, nivolumab, and tocilizumab combined with stereotactic body radiotherapy in patients with refractory pancreatic cancer (TRIPLE-R). Eur. J. Cancer 180, 125-133 (2023).
92. Huang, J. et al. Combination of pembrolizumab and radiotherapy induces systemic antitumor immune responses in immunologically cold non-small cell lung cancer. Nat. Cancer 6, 1676-1692 (2025).
93. Piffkó, A. et al. Radiation-induced amphiregulin drives tumour metastasis. Nature 1-10 (2025) doi: 10.1038/s41586-025-08994-0.
94. Zhang, J. et al. Inflammation awakens dormant cancer cells by modulating the epithelial-mesenchymal phenotypic state. Proc. Natl. Acad. Sci. 122, e2515009122 (2025).

1. A composition for treating metastatic cancer in a subject in need thereof, comprising:
a) a therapeutically effective amount of an amphiregulin (AREG) inhibitor; and
b) a therapeutically effective amount of a radiotherapy agent.

2. The composition of claim 1, wherein the AREG inhibitor comprises one or more of an anti-AREG antibody or an antigen-binding fragment thereof, an anti-AREG antibody drug conjugate (ADC), a protein binder, a peptide, an RNA, an AREG siRNA, a small molecule, and heparin.

3. The composition of claim 2, wherein the AREG inhibitor comprises an anti-AREG antibody or an antigen-binding fragment thereof.

4. The composition of claim 1, wherein the radiotherapy agent comprises one or more of yttrium-90, iodine-131, samarium-153, lutetium-177, astatine-211, lead-212 with bismuth-212, radium-223, actinium-225, and thorium-227.

5. The composition of claim 1, further comprising a therapeutically effective amount of an epidermal growth factor receptor (EGFR) inhibitor.

6. The composition of claim 5, wherein the EGFR inhibitor comprises one or more of an anti-EGFR antibody or an antigen-binding fragment thereof, an anti-EGFR ADC, a protein binder, a peptide, an RNA, an EGFR siRNA, and a small molecule.

7. The composition of claim 6, wherein the EGFR inhibitor is a small molecule tyrosine kinase inhibitor (TKI) of the tyrosine kinase domain of EGFR (EGFR TKI).

8. The composition of claim 5, wherein the EGFR inhibitor is gefitinib.

9. The composition of claim 1, further comprising a therapeutically effective amount of a Cluster of Differentiation 47 (CD47) inhibitor.

10. The composition of claim 9, wherein the CD47 inhibitor comprises one or more of an anti-CD47 antibody or an antigen-binding fragment thereof, an anti-CD47 ADC, a protein binder, a peptide, an RNA, a CD47 siRNA, and a small molecule.

11. The composition of claim 1, further comprising a therapeutically effective amount of a Signal transducer and activator of transcription 3 (STAT3) inhibitor.

12. The composition of claim 1, further comprising a therapeutically effective amount of a Signal regulatory protein α (SIRPα) inhibitor.

13. A composition for treating metastatic cancer in a subject in need thereof, comprising:
a) a therapeutically effective amount of a tumor necrosis factor α converting enzyme (TACE) inhibitor; and
b) a therapeutically effective amount of a radiotherapy agent.

14. The composition of claim 13, further comprising a therapeutically effective amount of an AREG inhibitor.

15. A composition for treating metastatic cancer in a subject in need thereof, comprising:
a) a therapeutically effective amount of an AREG inhibitor; and
b) a therapeutically effective amount of an immune checkpoint inhibitor.

16. The composition of claim 15, wherein the immune checkpoint inhibitor is an antibody or an antigen binding fragment thereof that binds to PD-1, PD-L1, CTLA-4, or CD47.

17. The composition of claim 15, further comprising a therapeutically effective amount of a radiotherapy agent.

18. A composition for treating metastatic cancer in a subject in need thereof, comprising:
a) a therapeutically effective amount of an AREG inhibitor; and
b) a therapeutically effective amount of an epidermal growth factor receptor (EGFR) inhibitor.

19. A method for treating a tumor in a patient, comprising:
a) obtaining a sample from the patient;
b) performing an assay on the sample to determine a level of AREG in the sample;
c) determining that the AREG level is higher than a reference level; and
d) administering a therapeutically effective amount of an AREG inhibitor to the patient and/or
administering a therapeutically effective amount of a radiotherapy agent to the patient,
whereby the tumor is treated.

20. The method of claim 19, where in the sample comprises one or more of blood, plasma, and tissue.

21. The method of claim 20, wherein the sample is plasma.

22. The method of claim 19, wherein step d) comprises administering a therapeutically effective amount of an AREG inhibitor to the patient and administering a therapeutically effective amount of a radiotherapy agent to the patient.

23. The method of claim 21, further comprising:
e) performing an assay on the sample to determine a level of circulating plasma EGFR in the sample;
f) determining that the EGFR level is higher than a reference level; and
g) administering a therapeutically effective amount of an EGFR inhibitor to the patient.

24. The method of claim 21, further comprising:
e) performing an assay on the sample to determine a level of circulating plasma CD47 in the sample;
f) determining that the CD47 level is higher than a reference level; and
g) administering a therapeutically effective amount of an CD47 inhibitor to the patient.

25. A method for treating a tumor in a patient, comprising:
a) obtaining a sample from the patient;
b) performing an assay on the sample to determine the level of circulating plasma AREG in the patient;
c) determining that the AREG level is higher than a reference level;
d) administering a therapeutically effective amount of an AREG inhibitor to the patient; and
e) administering a therapeutically effective amount of an immune checkpoint inhibitor to the patient,
whereby the tumor is treated.

26. A method for treating cancer in a patient, comprising:
a) obtaining a sample from the patient;
b) performing an assay on the sample to determine the level of circulating plasma AREG in a subject;
c) determining that the AREG level is higher than a reference level;
d) performing an assay on the sample to determine the level of circulating plasma EGFR in a subject;
e) determining that the EGFR level is higher than a reference level;
f) administering a therapeutically effective amount of an AREG inhibitor to the patient; and
g) administering a therapeutically effective amount of an EGFR inhibitor to the patient,
whereby the cancer is treated.

27. A method for treating a tumor in a patient, comprising:
determining if an AREG level in the patient is higher than a reference level by:
a) obtaining a sample from the patient;
b) performing an assay on the sample to determine the level of circulating plasma AREG in the patient;
if the sample has an AREG level higher than the reference level, then administering a therapeutically effective amount of an AREG inhibitor and a therapeutically effective amount of a radiotherapy agent to the patient, or
if sample has an AREG level lower than the reference level, then administering a therapeutically effective amount of a radiotherapy agent to the patient.

28. The method of claim 27, further comprising:
c) determining if an EGFR level is higher than a reference level by performing an assay on the sample to determine the level of circulating plasma EGFR in the patient;
if the sample has an EGFR level higher than the reference level, then administering a therapeutically effective amount of an EGFR inhibitor to the patient.

29. The method of claim 27, further comprising:
c) determining if a CD47 level is higher than a reference level by performing an assay on the sample to determine the level of circulating plasma CD47 in the patient;
if the sample has a CD47 level higher than the reference level, then administering a therapeutically effective amount of a CD47 inhibitor to the patient.

30. A kit, comprising:
a) a first assay for determining a concentration of AREG in a sample; and
b) a second assay for determining a concentration of EGFR in the sample; and/or
c) a third assay for determining a concentration of CD47 in the sample.

31. The kit of claim 30, wherein the first assay, second assay, and third assay each independently comprises an ELISA, a sandwich immunoassay with electrochemiluminescence, a bead-based immunoassay, and/or a proximity extension assay.


SEQUENCE LISTING (4 sequences)

SEQ ID NO: 1 - DNA, 21 bp, synthetic construct
  source 1..21: mol_type = other DNA
ggtcttaggctcaggccatta

SEQ ID NO: 2 - DNA, 21 bp, synthetic construct
  source 1..21: mol_type = other DNA
cgcttatggtggaaacctctc

SEQ ID NO: 3 - DNA, 20 bp, synthetic construct
  source 1..20: mol_type = other DNA
catggccctcttctgatttc

SEQ ID NO: 4 - DNA, 25 bp, synthetic construct
  source 1..25: mol_type = other DNA
ggaggttgtatagtcttctgattgg